(19) United States
(12) Patent Application Publication
(10) Pub. No.: US 2024/0301381 A1
(43) Pub. Date: Sep. 12, 2024

(54) ACE2-RECEPTOR ECTODOMAIN FUSION MOLECULES AND USES THEREOF

(71) Applicant: National Research Council of Canada, Ottawa (CA)

(72) Inventors: Traian SULEA, Kirkland (CA); Yves DUROCHER, Montreal (CA)

(73) Assignee: National Research Council of Canada, Ottawa, ON (CA)

(21) Appl. No.: 18/261,854

(22) PCT Filed: Jan. 25, 2022
(86) PCT No.: PCT/IB2022/050650

§ 371 Date: Jul. 18, 2023

Related U.S. Application Data
(60) Provisional application No. 63/141,594, filed on Jan. 26, 2021.

(51) Int. Cl. C12N 9/48; A61P 31/14; C12N 15/63

(52) U.S. Cl. CPC C12N 9/485; A61P 31/14; C12N 15/63; C12Y 304/17023

(57) ABSTRACT

The present invention relates, in general, to polypeptides capable of neutralizing SARS-CoV-2 and providing ACE2 enzymatic activity, and uses of these polypeptides for treating disorders related coronaviral infections (COVID-19) and the accompanying acute respiratory distress syndrome (ARDS) and major organ injuries, and methods of making such molecules.

### FIELD OF THE INVENTION

The present invention relates to human ACE2 receptor ectodomain fusion molecules and uses thereof. More specifically, the present invention relates to protein fusion variants of human ACE2 receptor catalytic domain with structural elements of human IgG1 antibody framework and their use in reducing coronaviral infections (COVID-19) and the accompanying acute respiratory distress syndrome (ARDS).

### BACKGROUND OF THE INVENTION

The COVID-19 pandemic continues as a world-wide health care crisis of unprecedented severity. Although clinical trials of potential vaccines are underway, uncertainty exists with regards to safety and efficacy of widespread population vaccination. Biotherapeutics for COVID-19 infection are desperately lacking, with only modest impacts of current strategies on patient outcomes. In this regard, many of the critical illness complications of COVID-19, including septic shock, acute respiratory distress syndrome (ARDS), and acute kidney injury (AKI) are mediated at least partly by the host response, especially within the renin-angiotensin system (RAS) [1-3]. Importantly, SARS-CoV-2, the virus causing COVID-19, interacts directly with angiotensin-converting enzyme 2 (ACE2), the plasma membrane protein that mediates its cellular entry [4-6].

The SARS-CoV-2 virus utilizes its spike protein for attachment and internalization into host cell as a pivotal step required for viral replication. Hence, the spike protein has become the principal molecular target for the development of promising anti-COVID-19 biotherapeutics, vaccines and diagnostic agents. The spike protein trimer in its prefusion state allows its receptor binding domain (RBD) to directly interact with the ACE2 receptor on the host cell. Since the emergence of this pandemic, much has been learned about the structure and function of the spike protein in relation to its human receptor ACE2. Some of this understanding has led to several approaches aiming to develop biotherapeutics against this disease. Among these many approaches, two main directions to block viral entry include binding to the spike protein with neutralizing monoclonal antibodies and with ACE2 receptor decoys. Strictly from the viral entry blockade point of view, the main liability of antibodies is the risk of decreased efficacy against emerging virus strains [7], whereas receptor decoys based on human ACE2 should in principle possess a robust pan-specificity profile.

Angiotensin (Ang) II is a potent vasoconstrictor with inflammatory and pro-coagulant actions. ACE2 is a mono-carboxypeptidase that converts Ang-I I to Ang-(1-7), a vasodilating counter-regulatory peptide to Ang-II. SARS-CoV-2 infection increases lung and coronary microvascular thrombosis and coagulation (increased D-dimers), which is associated with increased COVID-19 mortality [8, 9]. As for the respiratory viruses H1N1 and H5N1, SARS-CoV-2 binds and inhibits ACE2 [4-6] and therefore ACE2 is a potential biomarker and therapeutic target in patients with COVID-19 infection. ACE2 is downregulated in H1N1, H5N1, H7N9, and SARS leading to increased Ang-II levels, and worsened lung injury [10, 11]. Thus, local activation of the renin-angiotensin system (RAS) may mediate lung, cardiac and other organ injury responses to SARS-CoV-2 in COVID-19. Strategies to increase local tissue ACE2 activity in SARS-CoV-2 could therefore mitigate cell injury by reducing Ang-II (detrimental) and enhancing Ang-(1-7) (protective). Hence, ACE2-based decoys against this virus have the potential of not only neutralizing viral entry into the host cell, but via a dual mechanism of action, provide enzymatic conversion of Ang-II to Ang-(1-7), thereby shifting to restore the protective RAS pathway, and mitigate ARDS.

Several studies focused on using human ACE2 ectodomain alone or fused to the Fc region of human antibodies, with or without retention of its enzymatic activity [12-15]. However, innovative molecular engineering aimed at multi-factorial optimization is required to develop ACE2-based biotherapeutics with clinical efficiency and large-scale manufacturability. There remains a need for ACE2-based decoys that result in more effective virus neutralization while providing ACE2 enzymatic activity of Ang-II conversion.

### SUMMARY OF THE INVENTION

The present invention provides improved ACE2-based decoys. Unlike other approaches in this art, we have focused on a natural variant of the human ACE2 receptor that possesses an isoleucine (Ile) amino-acid residue at position 92, where a threonine (Thr) amino-acid residue is normally found in the more common ACE2 receptor broadly present in human population. We discovered, during the course of this invention, that employing structural and functional elements of this naturally occurring human variant, referred to herein as hACE2I92, improves several properties of ACE2-based decoys, among which the most critical for the aforementioned dual mechanism of action are improved catalytic activity and improved virus neutralization. Using ACE2I92 as a starting point in a multi-faceted molecular design effort, we now provide a class of polypeptide constructs which possess: (a) strong binding avidity and affinity to the spike protein for efficient neutralization of viral infection, (b) high enzymatic activity for reduced ARDS and (c) improved bio-manufacturability; while providing structural components for: (d) appropriate pharmacokinetics in order to allow viral clearance while preventing antibody dependent enhancement (ADE), and (e) protection against emerging strains and future pandemics.

Accordingly there is provided a polypeptide construct capable of neutralizing SARS-CoV-2 and converting Ang-II to Ang-(1-7) comprising four regions and having the general formula:

R1[hACE2I⁹²(18-614),X²⁷,X²⁶¹X³³⁰]-R2-R3[HingeS²²⁰,X²²⁶,X²²⁹]-R4[C_{H}2G²⁷⁰-C_{H}3]

and where: R1, denoted hACE2I⁹²(18-614),X²⁷,X²⁶¹X³³⁰, is the N-terminal first region comprising the naturally occurring variant Ile92 (192) of the human angiotensin converting enzyme 2 (hACE2) receptor catalytic domain residues 18 to 614 comprising residues X²⁷, X²⁶¹ and X³³⁰; wherein X²⁷ is the amino-acid residue at position 27 that is either Thr or Tyr, X²⁶¹ is the amino-acid residue at position 261 that is either Cys or Ser, and X³³⁰ is the amino-acid residue at position 330 that is either Asn or Tyr; R2 is a second region comprising a flexible peptide spacer; R3 is a third region comprising the hinge region of a human IgG1 heavy chain antibody, wherein said hinge region comprises residues S²²⁰, X²²⁶ and X²²⁹; wherein the amino acid residue at human IgG1 hinge position 220 is Ser, and wherein the amino acid residues at human IgG1 hinge positions 226 and 229 are either Cys or Ser; and R4 is a fourth region at the C-terminus of the polypeptide wherein R4 is denoted as and comprises C_{H}2G²⁷⁰-C_{H}3, and comprises the second constant domain (C_{H}2) and the third constant domain (C_{H}3) of human IgG1 antibody heavy chain, where the amino acid residue at position 270 in the human IgG1 C_{H}2 domain is Glycine.

In an embodiment, the R2 spacer region of the polypeptide construct comprises a flexible peptide spacer comprising Gly and Ser residues.

In an embodiment, the polypeptide construct neutralizes the SARS-CoV-2 with an IC50 of at least 500 ng/mL.

In an embodiment, the polypeptide construct retains at least 30% of the catalytic efficiency (k_{cat}/K_{M}) and at least 60% of the specific activity of the recombinant human ACE2.

In an embodiment, R1 of the polypeptide construct comprises a sequence selected from a group consisting of SEQ ID NO:2, SEQ ID NO:4, SEQ ID NO:5, SEQ ID NO:6, SEQ ID NO:7 and/or any sequence at least 90% identical thereto.

In an embodiment, R2 of the polypeptide construct comprises a sequence selected from a group consisting of SEQ ID NO:8, SEQ ID NO:9, and/or any sequence at least 90% identical thereto.

In an embodiment, R3 of the polypeptide construct comprises a sequence selected from a group consisting of SEQ ID NO:11, SEQ ID NO:12, and/or a sequence at least 90% identical thereto.

In an embodiment, R4 of the polypeptide construct comprises a sequence having SEQ ID NO:14, or a sequence at least 90% identical thereto.

In an embodiment, the polypeptide construct of the present invention comprises a sequence selected from a group consisting of SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:28, and a sequence at least 90% identical thereto.

In an embodiment, the polypeptide construct of the present invention is a dimeric polypeptide. In an embodiment, the dimeric polypeptide may be linked or may dimerize via the respective R3 hinge regions by disulfide bridges.

Another embodiment is a nucleic acid molecule encoding any polypeptide construct described herein. The present invention also provides an expression vector for producing polypeptides, wherein the expression vector comprises a nucleic acid molecule encoding any polypeptide construct described wherein. In an embodiment, the nucleic acid sequence that encodes a polypeptide of the present invention is in a form that is secretable by a selected expression host.

Another embodiment is a composition comprising a polypeptide construct described herein and a pharmaceutically-acceptable carrier, diluent, or excipient.

In another embodiment, there is provided a transgenic cellular host comprising the nucleic acid molecule encoding any polypeptide construct described herein, or an expression vector for producing any polypeptide constructs of the present invention.

In another embodiment, the transgenic cellular host further comprises a second nucleic acid molecule or a second vector encoding a second polypeptide construct the same as the first polypeptide construct.

Another embodiment is a method for producing a dimeric polypeptide comprising culturing the provided transgenic cellular host and recovering from medium conditioned by the growth of that host a dimeric polypeptide construct according to the present invention.

Another embodiment is a use of a polypeptide construct described herein for treatment of a medical condition, disease or disorder. In an embodiment, the medical condition, disease or disorder comprises coronaviral infections such as COVID-19, the acute respiratory distress syndrome (ARDS) and associated major organ failures such as of lung, heart, kidney, brain and intestine.

As described herein, the class of polypeptide constructs of the present invention comprises four regions R1, R2, R3 and R4 (FIG. 1). These polypeptides are useful for neutralizing SARS-CoV-2 to treat COVID-19, as well as converting Ang-II into Ang-(1-7) to treat ARDS. The polypeptides of the present invention have the general formula:

R1-R2-R3-R4

wherein R1 comprises hACE2I⁹²(18-614),X²⁷,X²⁶¹,X³³⁰, wherein X²⁷ is Thr or Tyr, X²⁶¹ is Cys or Ser, X³³⁰ is Asn or Tyr;

R2 comprises a spacer or linker;

R3 comprises a Hinge S²²⁰,X²²⁶,X²²⁹; wherein X²²⁶ and X²²⁹ is Cys or Ser; and

R4 comprises C_{H}2G²⁷⁰-C_{H}3.

In a preferred embodiment, a polypeptide comprises a polypeptide having the general formula:

R1[hACE2I⁹²(18-614),X²⁷,X²⁶¹,X³³⁰]-R2[Spacer]-R3[HingeS²²⁰,X²²⁶,X²²⁹]-R4[C_{H}2G²⁷⁰-C_{H}3]

The region R1, denoted hACE2I⁹²(18-614),X²⁷,X²¹⁶,X³³⁰, is a first (N-terminal) region comprising the naturally-occurring variant Ile92 of the human angiotensin converting enzyme 2 (hACE2I92). The ACE2 collectrin (neck) domain of the native ACE2 enzyme was determined to lock the ACE2 catalytic domain dimer in a rigid conformation, as this was incompatible with binding to the SARS-CoV-2 spike trimer; the neck (collectrin) domain was therefore removed, to generate the R1 region consisting of the receptor catalytic domain comprised of residues 18 to 614, and where X²⁷ is the amino-acid residue at position 27, that is either Thr or Tyr, X²⁶¹ is the amino-acid residue at position 261 that is either Cys or Ser, and X³³⁰ is the amino-acid residue at position 330 that is either Asn or Tyr. In preferred non-limiting embodiments, the R1 region of the polypeptide construct is selected from a group consisting of SEQ ID NO:2, SEQ ID NO:4, SEQ ID NO:5, SEQ ID NO:6, SEQ ID NO:7 and a sequence substantially identical thereto.

The region R2, also denoted herein as a spacer or linker, is a second region of a polypeptide construct of the present invention comprising a flexible polypeptide linker made of Gly and Ser residues. In preferred non-limiting embodiments, the R2 region of the polypeptide construct is selected from a group consisting of SEQ ID NO:8, SEQ ID NO:9, and a sequence substantially identical thereto. As would be understood by one of skill in this art, the polypeptide construct of the present invention is not limited to the R2 regions specifically noted herein, but may comprise any suitable spacer or linker (used herein interchangeably), provided that said linker or spacer is of a sequence and length that allows for the operable function of a polypeptide of the present invention.

The region R3, denoted HingeS²²⁰,X²²⁶,X²²⁹, is the third region comprising the hinge region of the human IgG1 heavy chain antibodies bearing a Ser amino-acid residue at position 220, and where X²²⁶ X²²⁹ are the amino-acid residues at positions 226 and 229, which are either Cys or Ser. In preferred non-limiting embodiments, the R2 region of the polypeptide construct is selected from a group consisting of SEQ ID NO:11, SEQ ID NO:12, and a sequence substantially identical thereto.

The region R4, denoted C_{H}2G²⁷⁰-C_{H}3, is the C-terminal region of the polypeptide construct comprising the second constant domain (C_{H}2) and the third constant domain (C_{H}3) of human IgG1 antibody heavy chain, where the CH2 domain contains a Gly amino-acid residue at position 270. In preferred non-limiting embodiments, the R2 region of the polypeptide construct has the sequence of SEQ ID NO:14 or any sequence substantially identical thereto.

In preferred non-limiting embodiments, the polypeptide construct of the present invention is selected from a group consisting of SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:28, and a sequence substantially identical thereto.

The polypeptide constructs provided in these preferred non-limiting embodiments can be produced at high yield by transient transfection in CHO cells, can be purified by Protein-A affinity chromatography and preparative size-exclusion chromatography (SEC) to high purity, possess elevated enzymatic activity, bind with high affinity and avidity to the spike protein of SARS-CoV-2, neutralize pseudo-typed SARS-CoV-2 with high potency, and neutralize the authentic SARS-CoV-2 virus in cellular and animal models. The class of polypeptide constructs described in this invention will be useful to reduce virus loads in living organisms, e.g., mice, hamsters and monkeys as animal models of disease, and humans for clinical applications. These compounds thus represent useful biotherapeutic agents for treating coronaviral infections including SARS and COVID-19 and their emerging strains, as well as viral-disease associated ARDS and injuries of multiple organs, e.g., lung, heart, kidney, brain and intestine.

Moreover, the ACE2 replacement function of these compounds provides additional therapeutic applications in other virally-induced pathologies resulting in Acute Respiratory Distress Syndrome (ARDS), such as Respiratory Syncytial Virus, Avian H5N1 Influenza, or due to sepsis-induced ARDS/cytokine storm. Finally, additional therapeutic indications include non-viral indications such as cardiac dysfunction as in myocarditis, perivascular and myocardial fibrosis, diabetic nephropathy, renal fibrosis and hepatic dysfunction resulting in NASH/NAFLD.

These and other features of the invention will now be described by way of example, with reference to the appended drawings:

### BRIEF DESCRIPTION OF THE DRAWINGS

FIG. 1 is a schematic diagram showing the design of the class of polypeptides of this invention comprising four regions R1, R2, R3 and R4, with reference to SEQ ID NOS in the sequence listing. Amino-acid position numbers indicated correspond to conventional numbering of each region and not sequential numbering along the full-length polypeptide sequence.

FIG. 2 illustrates the main principles of structure-based modular design leading to the class of polypeptides of the present invention. The ACE2 collectrin (neck) domain locks the ACE2 catalytic domain dimer in a rigid conformation incompatible with CoV-2 spike trimer; the neck (collectrin) domain was therefore removed. Introduced flexibility provided by the spacer and hinge regions unlocks ACE2 catalytic domain dimer for avid binding to CoV-2 spike trimer.

FIG. 3 presents a 3D rendering of the R1 region of the polypeptides of this invention (shown as dark gray ribbon), which was affinity-matured for improved binding to SARS-CoV-2 spike protein receptor binding domain (RBD) (shown as translucent molecular surface). The natural variant of human ACE2 catalytic domain carrying the T92I mutation represents the starting point for structure-based affinity maturation predictions carried out in this study with the ADAPT platform. Mutations selected in this invention for improved binding affinity to SARS-CoV-2 spike-RBD are rendered as CPK models at positions 27 and 330 of the ACE2I92 natural variant. See Table 1 for the top-30 binding affinity improving mutations according to ADAPT calculations.

FIGS. 4A, 4B and 4C presents the analysis of polypeptide variants produced by transient transfection in CHO cells and purified by Protein-A affinity chromatography. FIG. 4A provides SDS-PAGE analysis of denatured proteins under non-reducing and reducing conditions and different elution buffers. FIG. 4B and FIG. 4C provide UPLC-SEC analysis of eluted fractions for selected variants eluted from the Protein-A column with citrate pH 3.6 buffer and acetate pH 3.7 buffer, respectively. Molecular weights of the main peaks determined by MALS analysis are indicated.

FIGS. 5A, 5B and 5C presents the analysis of polypeptide variants following preparative size-exclusion chromatography. FIG. 5A provides SDS-PAGE analysis of denatured proteins under non-reducing and reducing conditions. FIG. 5B provides UPLC-SEC analysis of selected purified variants, with the molecular weight of the main peak determined by MALS analysis. FIG. 5C provides sedimentation velocity analytical ultra-centrifugation (AUC) analysis data for selected purified variants. Purity levels for a set of additional polypeptide variants are listed in Table 2.

FIGS. 6A, 6B, 6C, 6D and 6E presents enzymatic activity data for selected polypeptide variants. Recombinant human ACE2 (rhACE2) ectodomain is used as control. FIG. 6A provides the enzymatic activity determined using a fluorogenic substrate-based cell-free assay as function of substrate concentration at fixed enzyme concentration of 100 ng/mL used to determine the catalytic efficiency (k_{cat}/K_{M}) values. FIG. 6B provides activity as function of enzyme concentration used to determine specific activity values determined using a fluorogenic substrate-based cell-free assay. See Table 4 for enzymatic activity data for additional tested variants. FIG. 6C shows enzymatic activity assessed by hydrolysis of angiotensin II (Ang II) to angiotensin-(1-7) assayed by ELISA, of polypeptide variants of this invention or control enzymes (rhACE2 and rhACE) at 100 ng/ml, for 30 min incubation with 25 nM Ang II. Data presented as Mean±SEM, *P<0.01 vs rhACE. N=3-4 experiments. FIG. 6D presents fluorescence readings presented as RFU for enzymatic activity with 11.25 μM Mca-APK(Dnp) fluorogenic substrate for 30 min, in presence of mouse primary proximal tubular epithelial cells (PTECs) treated with 0.6 nM of polypeptide variants of this invention or control enzymes (rhACE2 and rhACE) for 24 h. Data presented as Mean±SEM, *P<0.01 vs rhACE. N=3 experiments. FIG. 6E presents fluorescence readings presented as RFU for enzymatic activity assessed by hydrolysis of Ang II (5 nM) to angiotensin-(1-7) (ELISA), in presence of mouse primary proximal tubular epithelial cells (PTECs) treated with 0.6 nM of polypeptide variants of this invention or control enzymes (rhACE2 and rhACE) for 24 h. Data presented as Mean±SEM, *P<0.01 vs rhACE. n=3 experiments.

FIGS. 7A, 7B, 7C, 7D and 7E presents the evaluation of binding ability of polypeptide variants of this invention to SARS-CoV-2 spike-RBD. FIG. 7A provides normalized SPR sensorgrams used to rank polypeptide constructs by dissociation rates (k_{off}). Homo-bivalent variants were flowed over immobilized spike-RBD of Wuhan SARS-CoV-2, which prevents calculation of binding dissociation constants due to avidity effects. FIG. 7B provides surrogate neutralization (sn)-ELISA binding competition data using immobilized spike-RBD of Wuhan SARS-CoV-2 and detection of bound biotinylated ACE2 by streptavidin-polyHRP. An IgG antibody isotype is used as a negative control. Table 5 lists SPR-based k_{off} dissociation rates and snELISA IC90 values of tested variants. FIG. 7C shows dissociation rates of polypeptides of this invention from spike-RBDs of SARS-CoV-2 variants Wuhan and B.1.351 (Beta) as well as SARS-CoV-1. FIGS. 7D and 7E provides SPR sensorgrams from long dissociation kinetics experiments of select polypeptides of this invention binding to spike-RBD of SARS-CoV-2 variants Wuhan, B.1.351 (Beta) and B.1.1.529 (Omicron), and the corresponding fitted apparent kinetic and equilibrium constants. Data presented as Mean±SD from N=3 experiments.

FIGS. 8A, 8B and 8C shows neutralization data based on a pseudo-typed lentiviral particle as a substitute for the live SARS-CoV-2 virus. The ability of polypeptide constructs of this invention to block entry of the pseudo-virus particle into a host cell line expressing ACE2 was measured. The pseudo-typed lentiviral particle contains the SARS-CoV-2 spike protein, a luciferase reporter and the minimal set of lentiviral proteins required to assemble the virus-like particle. Blockage of viral entry is detected by loss of signal of the luciferase reporter. FIG. 8A provides assay implementation 1, co-expressing human ACE2 and TMPRSS2 on HEK293T cells. FIG. 8B provides assay implementation expressing human ACE2 on HEK293T cells. See Table 6 for a listing of IC50 values of tested variants. FIG. 8C shows the ability of select polypeptides of this invention to neutralize cellular infection caused by viral like particles (VLPs) pseudo-typed with spike proteins from several SARS-CoV-2 variants of concern including Wuhan, D614G, B.1.1.7 (Alpha), B.1.351 (Beta) and B.1.617.2 (Delta). Associated IC50 values are listed in Table 6a.

FIG. 9 shows neutralization of live SARS-CoV-2 Wuhan virus for infecting VERO-E6 cells by select polypeptides of this invention. A neutralizing monoclonal antibody was used as positive control. Associated IC50 values are listed in Table 7.

FIGS. 10A, 10B, 10C and 10D presents the in vivo effect of select polypeptides of this invention after intravenous administration in hypertensive mice. FIG. 10A shows the effect on systolic blood pressure, measured by tail-cuff method 3, 6, 24, 48, and 72 h after administration of select polypeptides of this invention. FIG. 10B shows the ACE2 activity in plasma and various organs at 72 h after administration of select polypeptides of this invention (endpoint) assayed using the fluorogenic substrate Mca-APK(Dnp). FIG. 10C shows ELISA immunoblotting for human ACE2 and IgG-Fc in plasma and various organs at 72 h after administration of select polypeptides of this invention (endpoint). FIG. 10D shows the effects of treatments on albuminuria. ACR: urinary albumin to creatinine ratio. Data presented as Mean±SEM, P<0.05 @vs saline, *vs Ang II, ^{#}vs rhACE2, ^{&}vs Ang II+K, ^{%}vs Ang II+M. n=3 to 6 per group.

FIGS. 11A, 11B and 11C presents the in vivo therapeutic efficacy of select polypeptides of this invention after administration to hamsters infected with SARS-CoV-2 (Wuhan). FIGS. 11A and 11B shows body weight changes after intranasal administration as combined prophylactic and therapeutic dosing (FIG. 11A) or after therapeutic-only dosing (FIG. 11B). Male hamsters were challenged intranasally with 8×10¹ PFU of SARS-CoV-2 Wuhan isolate. Data plotted as Mean+/−SEM. n=6 to 7 per group. FIG. 11C shows live virus titers in lung tissues at day 3 post-infection after intravenous administration of combined prophylactic (−4 h) & therapeutic (+24 h) administration of 10 mg/kg of select polypeptides of this invention. Female hamsters were challenged intranasally with 10⁴ PFU of SARS-CoV-2 Wuhan isolate. Virus titre determined by plaque assay. Data plotted as Median+/−SEM. n=7 per group.

Additional aspects and advantages of the present invention will be apparent in view of the following description. The detailed descriptions and examples, while indicating preferred embodiments of the invention, are given by way of illustration only, as various changes and modifications within the scope of the invention will become apparent to those skilled in the art.

### DETAILED DESCRIPTION OF THE INVENTION

The present invention will be further illustrated in the following examples. However, it is to be understood that these examples are for illustrative purposes only and should not be used to limit the scope of the present invention in any manner.

The following therefore is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

The term “about” as used herein may be used to take into account experimental error, measurement error, and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm incorporated into the NBLAST and XBLAST programs [16]. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present invention. To obtain gapped alignments for comparison purposes, Gapped BLAST can be utilized as described in [17]. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller [18]. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

A “substantially identical” sequence may comprise one or more conservative amino acid mutations. It is known in the art that one or more conservative amino acid mutations to a reference sequence may yield a mutant peptide with no substantial change in physiological, chemical, physico-chemical or functional properties compared to the reference sequence; in such a case, the reference and mutant sequences would be considered “substantially identical” polypeptides. A conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pKa value of greater than 7, which are typically positively charged at physiological pH. Basic amino acids include arginine (Arg or R) and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of [19]. Hydrophobic amino acids include proline (Pro or P), isoleucine (lie or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pKa value of less than 7, which are typically negatively charged at physiological pH. Acidic amino acids include glutamate (Glu or E), and aspartate (Asp or D). Histidine (His or H) is a polar amino acid with a special ionization potential due to its pKa around 7, and more precisely around 6.4 in case of histidine residues located at the protein surface [20]. This results in histidine amino acid residues being a “polar” and predominantly uncharged at physiological pH of 7.2-7.4, and predominantly positively charged in acidic environments (pH<7).

The substantially identical sequences of the present invention may be at least 85% identical; in another example, the substantially identical sequences may be at least 85, 90, 91, 92, 93, 94, 95, 96, 97, 98, 99, or 100% identical, or any percentage there between, at the amino acid level or the nucleotide level to sequences described herein. Importantly, the substantially identical sequences retain the activity and specificity of the reference sequence. In a non-limiting embodiment, the difference in sequence identity may be due to conservative amino acid mutation(s). In a non-limiting embodiment, the difference in sequence identity may be due to synonymous nucleotide substitutions or nucleotide substitutions that give rise to conservative amino acid mutation(s). In a non-limiting example, the present invention may be directed to polypeptide construct comprising an amino acid sequence that is at least 85%, 90%, or 95% identical to the polypeptide construct sequence set forth in SEQ ID NO: 23.

As used herein the terms “peptide” and “polypeptide” refer to a linear chain of two or more amino acids joined by peptide bonds. The term “peptide” is generally used to refer to a short chain of amino acids comprising 2 to 49 amino acids, whereas the term “polypeptide” is generally used to refer to a longer chain of amino acids comprising 50 or more amino acids. However, these terms may be used interchangeably. The term “polypeptide construct” is used herein to refer to one or more peptides or polypeptides that have been folded and/or assembled to form a three-dimensional structure, although protein and polypeptide construct may also be used interchangeably. A protein may include post-translational modifications, as will be understood to one skilled in the art. For example, a protein may be glycosylated, lipidated, phosphorylated, ubiquitinated, acetylated, nitrosylated, and/or methylated.

As used herein, the term “recombinant polypeptide” refers to a polypeptide that is produced by recombinant techniques, wherein generally DNA or RNA encoding the expressed protein is inserted into a suitable expression vector that is in turn introduced into a host cell to allow expression of the recombinant polypeptide. Recombinant polypeptides may include amino acid sequences from two or more sources, such as different proteins. Such recombinant polypeptides may be referred to as fusion polypeptides. Recombinant polypeptides may also include one or more synthetic amino acid sequences.

As used herein, the term “spacer” or “linker” refers to a peptide that directly and covalently links two polypeptides. The linker may be an amino acid, or a peptide comprising two or more amino acids. If the linker is an amino acid or peptide, the N-terminal end of the linker may be covalently linked by a peptide bond to the C-terminal end of a first polypeptide and the C-terminal end of the linker may be covalently linked by a peptide bond to the N-terminal end of a second polypeptide. Typically, the two polypeptides covalently linked by the linker are polypeptides that are not naturally joined, for example they may be encoded by different genes and/or by different species, or they may be different portions or domains of a single polypeptide or protein. In a non-limiting example, the linker or spacer may be less than 20 amino acids.

As used herein, the term “antigen” refers to any molecule, moiety or entity that is capable of eliciting an immune response. This includes cellular and/or humoral immune responses. An antigen is commonly a biological molecule, usually a protein, peptide, polysaccharide, lipid or conjugate that contains at least one epitope to which a cognate antibody can selectively bind.

A “viral surface antigen” is an antigen, such as a polypeptide, that can be found on the surface of a virus. The viral surface antigen may be a trimeric viral surface antigen. Examples of trimeric viral surface antigens include but are not limited to Severe Acute Respiratory Syndrome (SARS)-coronavirus (CoV)-2 (SARS-CoV-2) spike, SARS-CoV-1 spike, Middle East Respiratory Syndrome (MERS)-CoV spike, Influenza hemagglutinin (HA), human immunodeficiency virus (HIV) gp120, Respiratory syncytial virus (RSV) RSVF protein, the Rabies Virus Glycoprotein (RABVG), and the Human metapneumovirus (hMPV) glycoprotein.

As used herein, the term “pharmaceutically acceptable carrier” refers to a carrier that is non-toxic. Suitable pharmaceutically acceptable carriers include, for example, one or more of water, saline, phosphate buffered saline, dextrose, glycerol, ethanol, and combinations thereof. Pharmaceutically acceptable carriers may further contain minor amounts of auxiliary substances such as wetting or emulsifying agents, preservatives or buffering agents that enhance shelf life or effectiveness.

As used herein, the term “fragment”, in reference to a molecule, such as a nucleic acid molecule or a polypeptide, refers to a portion of the molecule that is less than the full length of the molecule.

As used herein, the term “subject” refers to a human or non-human animal, for example a mammal, avian, reptile, fish, or amphibian.

As used herein, the terms hinge fragment, C_{H}2 domain and C_{H}3 domain refer to the corresponding regions of the IgG antibody heavy chain, having nucleotide and protein sequences as defined and numbered according to the ImMunoGeneTics (IMGT) database (http://www.imgt.org/) [21-23]. Preferred non-limiting embodiments of the hinge fragment, C_{H}2 domain and C_{H}3 domain are from a human antibody, and preferably the human IgG1 isotype.

It should also be understood that, in certain methods described herein that include more than one step or act, the order of the steps or acts of the method is not necessarily limited to the order in which the steps or acts of the method are recited unless the context indicates otherwise.

To address the problems in the art, the present invention provides an improved ACE2-based decoy variant of the human ACE2 receptor that possesses an isoleucine (lie) amino-acid residue at position 92, where a threonine (Thr) amino-acid residue is normally found in the more common ACE2 receptor broadly present in human population. The present invention uses ACE2I92 as a starting point in a multi-faceted molecular design effort, which now provides a class of polypeptide constructs which possess: (a) strong binding avidity and affinity to the spike protein for efficient neutralization of viral infection, (b) high enzymatic activity for reduced ARDS and (c) improved bio-manufacturability; while providing structural components for: (d) appropriate pharmacokinetics in order to allow viral clearance while preventing antibody dependent enhancement (ADE), and (e) protection against emerging strains and future pandemics.

The present invention provides polypeptide constructs having the general formula:

R1-R2-R3-R4

wherein R1 comprises hACE2I⁹²(18-614),X²⁷,X²⁶¹,X³³⁰, wherein X²⁷ is Thr or Tyr, X²⁶¹ is Cys or Ser, X³³⁰ is Asn or Tyr;

R2 comprises a spacer or linker;

R3 comprises a Hinge S²²⁰,X²²⁶,X²²⁹; wherein X²²⁶ and X²²⁹ is Cys or Ser; and

R4 comprises C_{H}2G²⁷⁰-C_{H}3,

where the structural features and designed advantages of each of the four regions are presented and described in detail in the following section entitled Example 1 “Molecular engineering of polypeptide constructs”. The experimental data demonstrating the advantages of designed polypeptide constructs of this invention are presented in the following section under Example 2 through Example 7. Non-limiting illustrative examples of the present disclosure are provided in SEQ ID NOS: 15-29, and more specifically SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:28.

### Example 1. Molecular Engineering of Polypeptide Constructs

### Structure-Based Modular Design for Optimization of Avidity to SARS-CoV-2 Spike Homotrimer

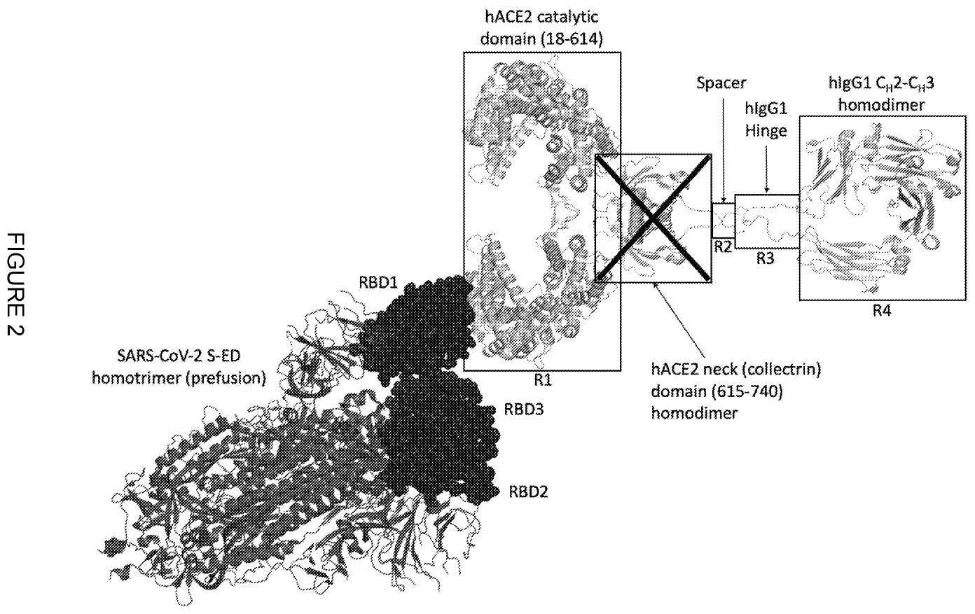

FIG. 2 illustrates structural domains utilized in the modular design of the polypeptides of the present invention. The molecular model of the SARS-CoV-2 spike homotrimer was taken from the cryo-EM structure with PDB ID 6VSB. Human ACE2 ectodomain (18-740) homodimer including catalytic and neck (collectrin) domains, in complex with the SARS-CoV-2 receptor binding domain (RBD), was taken from the cryo-EM structure with PDB ID 6M17. Human IgG1 Fc fragment homodimer including hinge, C_{H}2 and C_{H}3 domains was taken from the crystal structure of a human IgG1 mAb having PDB ID 1 HZH. Structural manipulation, visualization and rendering was done with the PyMOL Molecular Graphics System (Schroedinger, LLC).

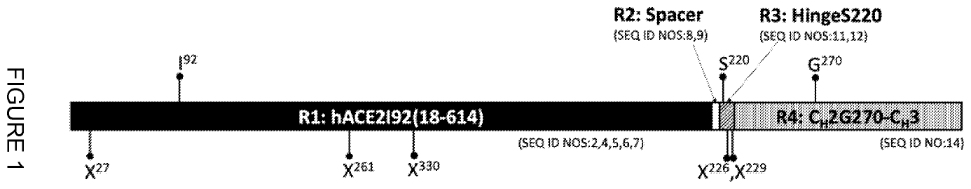

As a first step of this modular design, we determined that the neck (collectrin) domain acts as a non-covalent dimerization domain and locks the ACE2 catalytic domains (R1 region) in a rigid mutual orientation, which in this study we determined to be incompatible with simultaneous binding onto two RBD domains of a spike protein homotrimer, the neck domain was therefore removed, as shown in FIG. 2. The next step was to link the remaining human ACE2 catalytic domains (R1 region) via a flexible polypeptide spacer (R2 region) followed by the flexible human IgG1 hinge (R3 region) to a human IgG1 C_{H}2-C_{H}3 fragment (R4 region) that non-covalently assembles as a homodimer. This novel structure-based design strategy provides increased flexibility and mutual independence of the two ACE2 catalytic domains in the context of the homodimeric polypeptide constructs of this invention. The overall goal is to facilitate and improve simultaneous occupancy of the two R1 regions of the designed homo-dimeric polypeptide constructs of this invention on two RBD domains of the same viral spike protein homo-trimeric molecule, as well as to two RBD domains that belong to distinct spike protein homotrimers, adjacent to each other at the virion surface. Hence, the unique design platform of the polypeptide constructs of this invention was used to select residues that confer high binding avidity and affinity to the viral spike protein, as determined by ADAPT affinity maturation (see below). It is understood that the deletion of the collectrin (neck) domain is non-limiting, and employing the full-length hACE2(18-740) ectodomain in the design concepts described in FIG. 1 and FIG. 2 is an obvious modification that one of skill in this art would understand is able to retain significant levels of enzymatic activity and viral neutralization for the polypeptide constructs of this invention.

### Additional Sequence Optimization in Each of the Four Regions of the Polypeptide Constructs

The sequence variations described in this paragraph are indicated schematically in FIG. 1. First, in the ACE2 catalytic domain (R1 region), the naturally occurring human mutation T92I [24-26] was selected. This natural mutation is predicted to eliminate the N⁹⁰XT⁹² N-glycosylation sequon and hence N-glycosylation of Asn90. A first benefit of employing this partially de-glycosylated natural variant in our design is that it leads to lower molecular weights of the polypeptide constructs of this invention, which may improve tissue penetration. Furthermore, as it will be shown in this specification for the first time, the ACE2I92 partially de-glycosylated natural variant imparts to the polypeptide constructs of this invention improved activity profiles in terms of both increased enzymatic activity and increased virus neutralization potency. Importantly, the natural occurrence of the Ile amino-acid at position 92 of human ACE2 eliminates the immunogenicity concern that might arise due to protein surface exposure that results from removal of the carbohydrate structure at Asn90.

Two different lengths of the spacer (R2 region) were designed in order to evaluate the effect on flexibility and mutual freedom of the two independent ACE2 catalytic domains (R1 region) in the polypeptide construct homodimer; a 5-residue spacer SGGGG, and a 15-residue spacer SGGGGSGGGGSGGGG. Geometric measurements were made in PyMOL on molecular models to ensure that the shorter spacer allows the two independent ACE2 catalytic domains (R1 region) to be accommodated in the homodimeric polypeptide construct without steric hindrance.

The human IgG1 hinge (R3 region) was also modified. First, we mutated the unpaired Cys residue at hinge position 220 that is natively engaged in a disulfide bridge to the antibody light chain, which is not present in our constructs, thus aiming to reduce the possibility of forming undesired covalent multimeric species via Cys220, which was thus substituted by a Ser amino-acid. Furthermore, variants were designed that additionally substitute the remaining two Cys residues in the native human IgG1 hinge region with Ser residues at hinge positions 226 and 229. While these Cys residues normally form disulfide bridges between the two heavy chains in the Fc fragment homodimer, they can also lead to undesired covalent multimeric species that impact the manufacturability of Fc-fused proteins [27, 28]. A structural feature was sought to reduce sample heterogeneity during large-scale manufacturability. Hinge-mutated R3 regions devoid of Cys residues were conceived to lead to homo-dimeric variants, stabilized solely by non-covalent inter-polypeptide chain interactions, mainly via the C_{H}3 domain homodimerization, and thus not stabilized by inter-polypeptide chain covalent disulfide bonds. In the same spirit, certain embodiments of this invention also include the mutated surface-exposed, unpaired Cys residue at position 261 of the ACE2 catalytic domain (R1 region) to a Ser residue.

Finally, the human C_{H}2 domain from the R4 region was selected to include the mutation D270G, that may attenuate immune effector function via reduced binding to FcγR receptors and the Cq complement complex. This modification aims to reduce certain side-effects of antibody treatments of viral infection, namely: (i) the antibody-dependent enhancement (ADE) effect shown to exacerbate the pathology in certain viral infections [29], and (ii) increased inflammation of organs and tissues already affected by the acute respiratory distress syndrome (ARDS) due with COVID-19 infection [30-32].

### ADAPT Affinity Maturation Against SARS-CoV Spike RBD

In addition to the above-mentioned sequence improvements and modifications, the human ACE2 catalytic domain (R1 region) was further engineered by incorporation of residues identified by affinity maturation against the SARS-CoV-2 spike protein. The starting point for affinity maturation were the atomic coordinates of the hACE2 bound to SARS-CoV-2 spike protein receptor binding domain (RBD) which were taken from the cryo-EM structure with PDB ID 6M17 [4]. Only one copy of the human ACE2 catalytic domain residues 21-615 and SARS-CoV-2 spike RBD residues 336-518 were retained and all other atoms removed. Hydrogen atoms were added to the complex and adjusted for maximizing H-bonding interactions. Structural refinement of the complex was then carried out by energy-minimization using the AMBER force-field [33, 34] with a distance-dependent dielectric and an infinite distance cutoff for non-bonded interactions. Non-hydrogen atoms were restrained at their crystallographic positions with harmonic force constants of 40 and 10 kcal/(mol·A²) for the backbone and side-chain atoms, respectively. The ADAPT platform was then used for affinity maturation [35, 36]. Single-point scanning mutagenesis simulations were carried out at 57 positions within the ACE2 catalytic domain (R1 region) that may impact binding affinity to the spike RBD upon mutations. We used the ADAPT protocols for building the structures and evaluating the energies of single-point mutations to 17 other possible natural amino-acids (Cys and Pro were excluded) at these positions of the parental hACE2 structure. A consensus approach over specific versions of these three protocols [37] was applied for building and scoring the hACE2 mutants. Scoring of binding affinity was mainly based on the average Z-score over the scores calculated with the three component energy functions. Prior to binding affinity predictions, mutations predicted to destabilize the correct folding of the ACE2 catalytic domain were discarded from further evaluation.

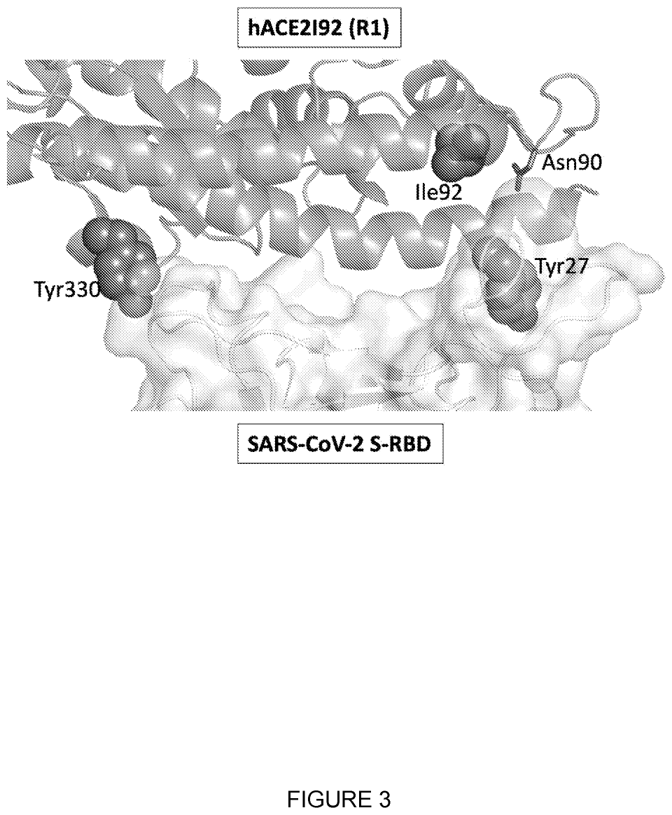

The selection of the most likely single-point mutants with improved spike-RBD binding affinities was primarily guided by the best 30 consensus-Z-scores from ADAPT (Table 1). Visual examination of the molecular interactions predicted with the three sampling protocols of ADAPT was used to detect sub-optimal complementarity at the antibody-interface (e.g., burial of polar groups in non-polar environments), steric overcrowding and distortions in covalent geometry (e.g., deviations from planarity of aromatic rings), and replacements of native Gly residues that have their main chain torsion angles in the disallowed region of the Ramachandran plot. Two positions were finally selected, Thr27 and Asn330, where aromatic substitutions were predicted with high probability to improve binding affinity relative to the parental ACE2 structure. Among those possible substitutions, we chose Tyr as substituting residue given its increased solubility relative to the other predicted replacements Phe and Trp (FIG. 3). In the polypeptide constructs of this invention, the T27Y and N330Y mutations were used either alone or in combination, and also preferably combined with the sequence variations described in the previous section.

TABLE 1
Consensus Z-scores for top-30 mutations of ACE2 catalytic domain predicted with the structure-
based affinity maturation platform ADAPT to improve binding to SARS-CoV-2 spike-RBD. Large
negative scores predict significant improvements of binding affinity upon mutation.

| Residue | R | K | N | S | T | H | W | Y | F | M | L | I | V | A | G | D |
|---|---|---|---|---|---|---|---|---|---|---|---|---|---|---|---|---|
| Thr27 | | | −3.8 | | | | −3.5 | −4.6 | −5.7 | | | | | | | −2.6 |
| Asn330 | | | | | | −2.7 | −5.5 | −5.2 | −5.4 | | −2.6 | | | | | |
| His34 | | | | | | | −5.5 | −3.4 | −4.3 | | | | | | | |
| Gln42 | −3.8 | −4.4 | −3.8 | −3.1 | −2.8 | −4.7 | −4.6 | −2.9 | −3.8 | −4.3 | −3.6 | −2.9 | −3.0 | −2.9 | −3.0 | |
| Leu45 | | | | | | | −4.0 | | | −2.7 | | | | | | |

### Example 2. Production of Designed Polypeptide Constructs in CHO Cells

The various polypeptide constructs of this invention include the native signal sequence of human ACE2 at their N-termini. The DNA coding regions for the constructs were prepared synthetically (GenScript) and were cloned into the HindIII (5′ end) and BamH1 (3′ end) sites of the pTT5 mammalian expression plasmid vector [38]. Fusion proteins were produced by transient transfection of Chinese Hamster Ovary (CHO) cells. Briefly, plasmid DNAs were transfected into a 0.5 L or 1 L cultures of CHO-55E1 cells. Transfections were performed at a cell density between 1.8×10⁶ and 2.0×10⁶ cells/mL with viability greater than 98%. Cells were distributed in 1.0 L to 2.8 L-shaker flasks and transfected with 1 μg of total DNA per 1 mL of production using PEI MAX™ (Polysciences, Inc., Warrington, PA). The final DNA: PEI MAX™ ratio was 1:4 (w/w). Cell cultures were incubated for 24 h on an orbital shaking platform at an agitation rate of 110 rpm at 37° C. in a humidified 5% CO₂ atmosphere. Twenty-four hours later, the cultures were fed with Tryptone N1 at 1% w/v final and Valproic acid sodium salt at 0.5 mM final concentration and transferred to 32° C. for 6 days. Cell density and cell viability were determined by direct counting of cell samples with a Vi-CELL automated cell counting system (Beckman Coulter Life Sciences, Indianapolis, IN) using the trypan blue dye exclusion method.

Polypeptide constructs were produces at yields between 104 and 328 mg/L, with typical yield around 300 mg/L for most variants. This demonstrates that the polypeptide constructs of this invention can be efficiently produced by transient transfection in CHO cells. Further yield increases are expected by selection and propagation of pools of CHO cells having the best expressions of particular variants.

### Example 3. Purification of Polypeptide Constructs by Affinity Chromatography

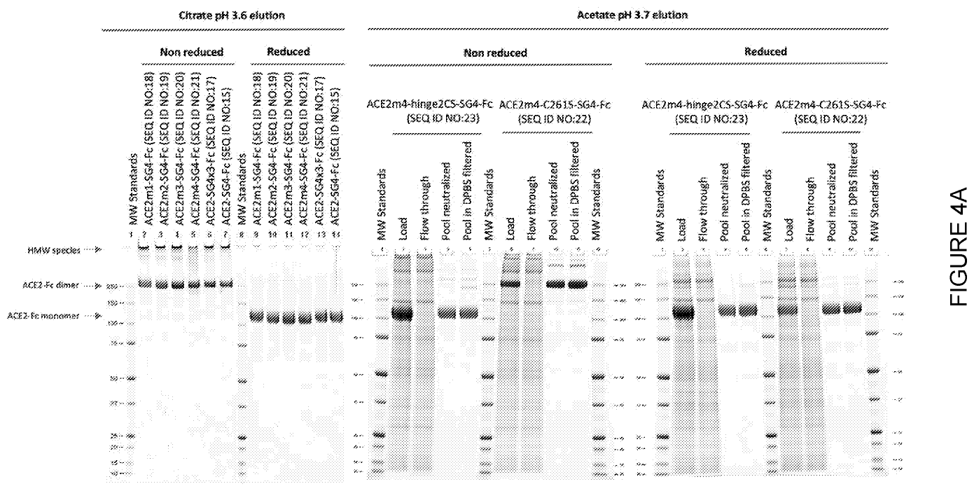

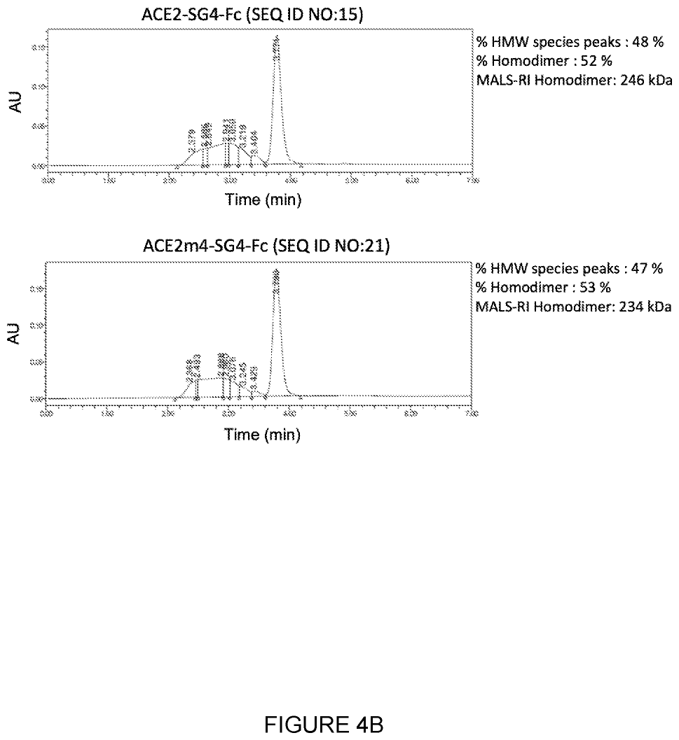

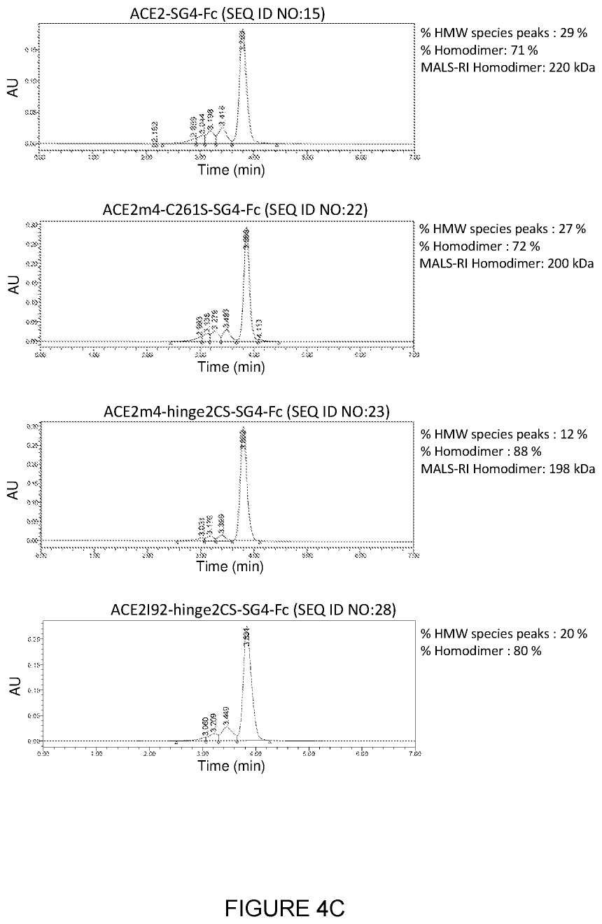

Purification from cell-culture supernatants was performed by Protein-A affinity chromatography. Cell-culture supernatants were loaded onto a 5 mL HiTrap MabSelect™ SuRe™ column (GE Healthcare Life Sciences) equilibrated in DPBS. Supernatants were loaded using linear flow rate for binding set at ˜45 cm/h (1.7 mL/min) to get a residence time of ˜2.9 min. The column was washed with DPBS and protein eluted with 0.1 M citrate buffer pH 3.6 or 0.1 M acetate buffer pH 3.7. Neutralization was done with 10% (v/v) 1 M HEPES. Neutralized elution pools were buffer-exchanged using Zeba spin columns and sterile-filtered. Using citrate pH 3.6 elution buffer, the tested samples of variants carrying cysteines at positions 226 and 229 of the hinge (R3 region) and position 261 of the ACE2 catalytic domain (R1 region) contained about 50% homodimer with the rest being aggregated material of high-molecular-weight (HMW) species according to SDS-PAGE tests under denatured non-reducing conditions, with most of HMW species disappearing under reducing conditions (FIG. 4A, left). This level of heterogeneity was confirmed by analytical UPLC-SEC on high-resolution BEH-450 column (FIG. 4B). However, using the acetate pH 3.7 as elution buffer improved homogeneity of preparations. As seen from the denatured SDS-PAGE data under non-reducing conditions in FIG. 4A (right) for the variants with mutated cysteines either at both hinge positions 226 and 229 (ACE2m4-hinge2CS-SG4-Fc; SEQ ID NO:23) or at position 261 of the ACE2 catalytic domain ACE2m4-C261S-SG4-Fc; SEQ ID NO:22), HMW species are not present. As expected, the ACE2m4-hinge2CS-SG4-Fc (SEQ ID NO:23) variant appears as a monomer under denatured non-reducing conditions due to the absence of covalent disulfides between the hinge regions of the native homodimer. Furthermore, analytical UPLC-SEC data on BEH-450 column shown in FIG. 4C suggests that the main factor of improved homogeneity of the purified samples the elution buffer (˜50% homodimer using the citrate pH 3.6 elution buffer versus ˜70% homodimer using the acetate pH 3.7 elution buffer) and no mutations of cysteine residues. This is clearly apparent from the variant ACE2-SG4-Fc (SEQ ID NO:15) tested with both elution buffers, which shows that the size and amount of HMW species is reduced with the acetate pH 3.7 elution buffer (FIG. 4C) versus citrate pH 3.6 elution buffer (FIG. 4B). We also observed that mutating the C261 to Ser in the ACE2 catalytic domain (R1 region) does not further improve homogeneity (using the acetate pH 3.7 elution buffer), which contains 72% homodimer. However, as a novel finding, the homogeneity improved significantly when the two Cys at positions 226 and 229 of the hinge (R3 region) were both mutated to Ser, to generate a 88% homodimer with only 12% HMW species of relatively smaller sizes (FIG. 4C). Moreover, the MW determined by a MALS-RI analysis for the homodimer species of this purified sample of the ACE2m4-hinge2CS-SG4-Fc (SEQ ID NO:23) variant is 198 kDa, which is in good agreement with the theoretical homodimer MW of 190 kDa, and considering that the difference is likely due to glycosylation. All these results indicate that using the acetate pH 3.7 elution buffer and mutating the cysteines at human IgG1 hinge positions 226 and 229 are important to attain a reasonably high homogeneity of approximately 90% by Protein-A purification for this class of polypeptides.

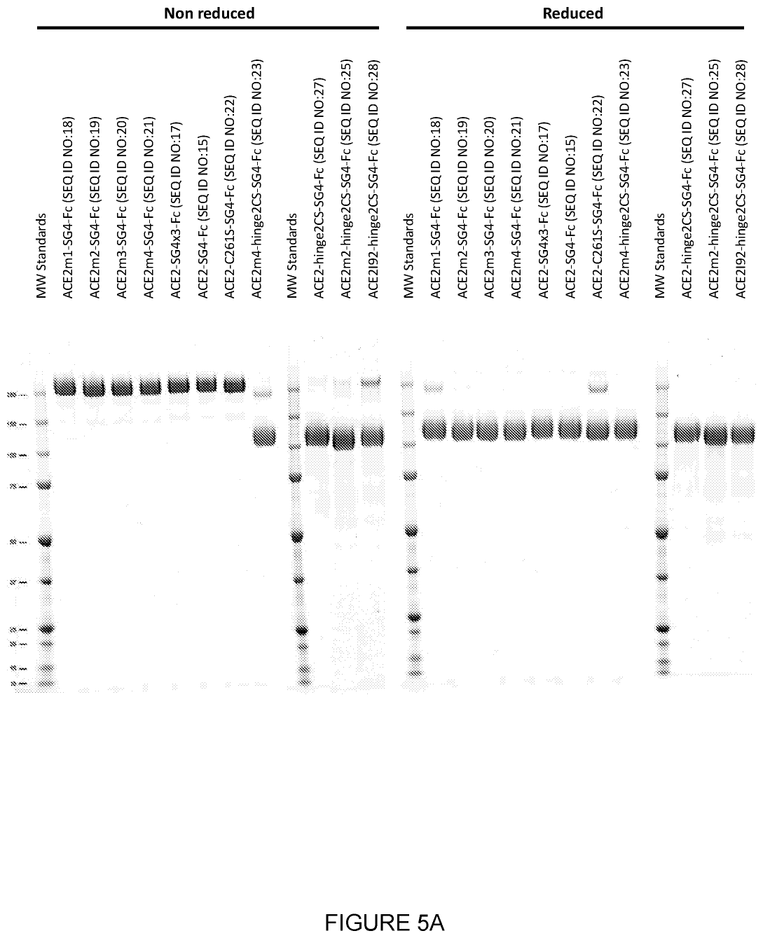

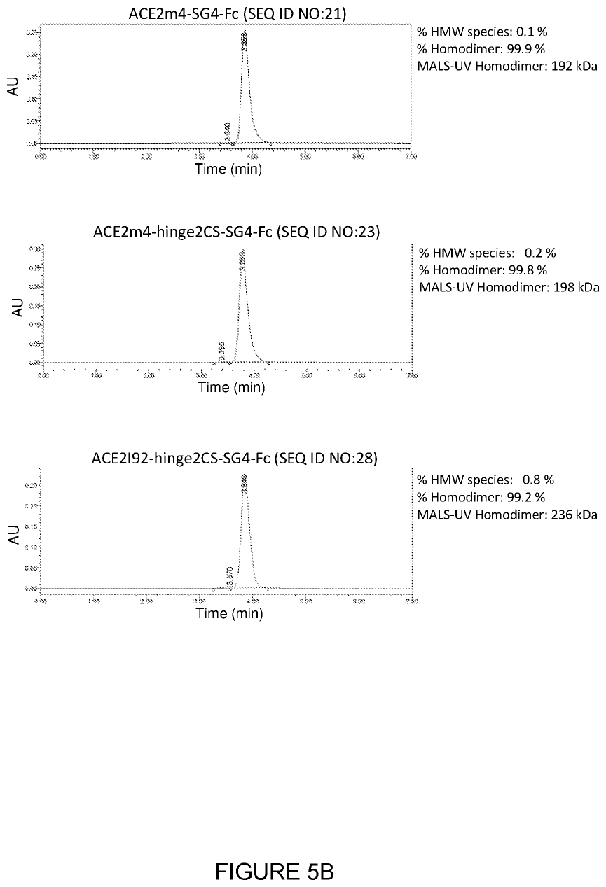

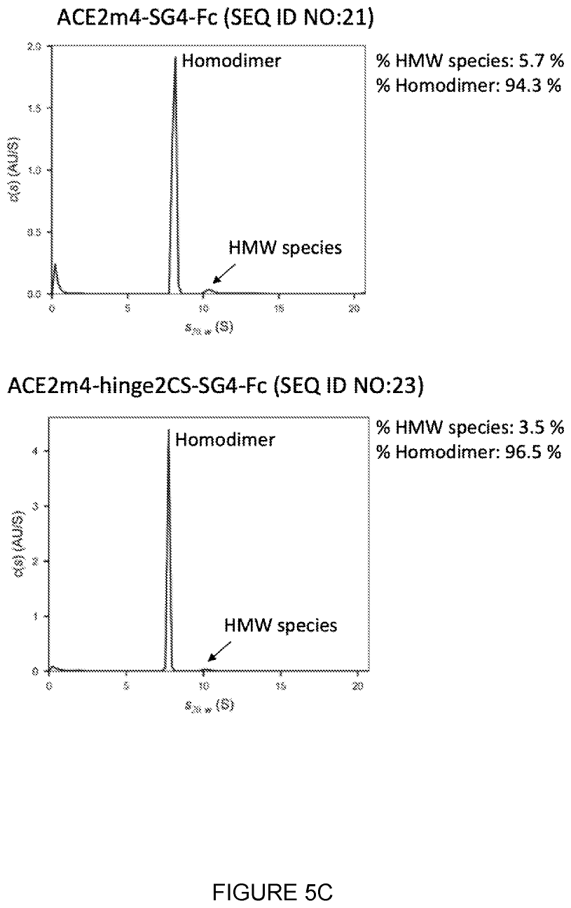

Next, all Protein-A purified samples were further purified by preparative SEC on a Superose 6 Increase column (GE Healthcare Life Sciences). Selected peak fractions were concentrated by ultrafiltration using Vivaspin® 6 centrifugal concentrators with a membrane molecular weight cut-off of 10 kDa (GE Healthcare Life Sciences) at 15° C. following the manufacturer's instructions. During the process, the protein concentration was monitored on a NanoDrop™ 2000 spectrophotometer (Thermo Fisher Scientific, Waltham, MA) using absorbance at 280 nm and the calculated specific extinction coefficient of each variant. The SDS-PAGE analysis of denatured purified variants indicate highly pure homodimers for all polypeptide constructs under both non-reducing and reducing conditions (FIG. 5A). The ACE2m4-hinge2CS-SG4-Fc (SEQ ID NO:23) variant appears as a monomer under denatured non-reducing conditions, due to the absence of covalent disulfides between the hinge regions of the native homodimer. High purities were then confirmed by analytical UPLC-SEC on high-resolution BEH-450 column, with representative chromatograms shown in FIG. 5B and homodimer fraction listed in Table 2 for all variants. It is important to note that both ACE2m4-SG4-Fc (SEQ ID NO:21) and ACE2m4-hinge2CS-SG4-Fc (SEQ ID NO:23) have almost identical UPLC-SEC chromatograms, both indicating homodimers, despite the difference between these variants being the presence (in the former variant) or the absence (in the latter variant) of inter-polypeptide chain disulfide bridges at the level of the hinge (R3 region). As indicated in FIG. 5B and Table 2, the MWs determined for the homodimers of these species are in close agreement with glycosylated proteins with calculated protein MW of 190 kDa. Notably, the determined MWs for the variants based on the natural variant of human ACE2 Ile92 that lacks the carbohydrate at position Asn90 (SEQ ID NOS: 19, 20, 21, 22 and 23) are smaller than for those with glycosylation at Asn90 (SEQ ID NOS: 15, 17 and 18), as shown in Table 2.

Samples purified by preparative SEC were further analyzed by sedimentation velocity analytical ultracentrifugation (SV-AUC) performed on a Beckman Proteomelab XL-I with AN-50 8-hole rotor, monitoring absorbance at 280 nm at a protein concentration of 1 mg/mL in PBS. Cells with 2 sector charcoal-epon centerpieces with 3 mm pathlength and sapphire windows were used. Proteins were centrifuged at 45000 rpm, and scans were performed every 4 min. The c(s) distributions were obtained using the SEDFIT software and integrated using GUSSI software. The SV-AUC data tabulated in Table 2 indicates the purified samples of the tested polypeptide constructs have one major peak at ˜8 S that accounts for 88-97% of the protein peak area and is attributed to the homodimer species. The content of HMW species is very low. Frictional coefficients of these samples range from 1.8-2.1, implying an asymmetric conformation for these homodimeric assemblies, which is consistent to the engineered conformational flexibility as described earlier. FIG. 5C shows similar sedimentation coefficient distributions for the pair of variants that differ only in the presence or absence of inter-polypeptide chain covalent disulfide bonds in the hinge (R3 region): ACE2m4-SG4-Fc (SEQ ID NO:21) versus ACE2m4-hinge2CS-SG4-Fc (SEQ ID NO:23).

TABLE 2
Homogeneity of purified variants.

| | | % homo- | | % homo- | Radius |
|---|---|---|---|---|---|
| | SEQ | dimer | MW | dimer | (S) |
| | ID | (UPLC- | (kDa) | (SV- | (SV- |
| Variant | NO | SEC) | (MALS) | AUC) | AUC) |
| ACE2-SG4-Fc | 15 | 99.9 | 202 | 92.8 | 8.252 |
| ACE2-SG4x3-Fc | 17 | 99.9 | 205 | 94.3 | 8.173 |
| ACE2m1-SG4-Fc | 18 | 99.7 | 201 | 88.4 | 8.220 |
| ACE2m2-SG4-Fc | 19 | 99.9 | 194 | 87.9 | 8.057 |
| ACE2m3-SG4-Fc | 20 | 99.9 | 192 | 96.0 | 8.067 |
| ACE2m4-SG4-Fc | 21 | 99.9 | 192 | 94.3 | 8.094 |
| ACE2m4-C261S- | 22 | 99.5 | 196 | 92.9 | 8.067 |
| SG4-Fc | | | | | |
| ACE2m4-hinge2CS- | 23 | 99.8 | 198 | 96.5 | 7.751 |
| SG4-Fc | | | | | |

### Example 4. Stability Characterization of Polypeptide Constructs

### Freeze-Thaw Cycles

Purified samples of the polypeptide constructs of this invention were subjected to freeze-thaw stability stress test, consisting of 3 cycles of freezing at −80° C. and thawing for 30 min. Analytical UPLC-SEC on BEH-200 column was used to determine sample heterogeneity before and after each of the freeze-thaw cycles. All samples were extremely resilient to the freeze-thaw stress. The fraction of homodimer determined after the 3^{rd} freeze-thaw cycle is listed in Table 3. Samples before stress and after the 3^{rd} freeze-thaw cycle were also tested for SARS-CoV-2 spike-RBD binding using SPR (vide infra). A similarity score comparing the SPR sensorgrams before and after freeze-thaw stress is recorded in Table 3 and indicates very similar of binding levels. Taken together, these data indicate that the polypeptide constructs of this invention are very resilient to freeze-thaw stress and can be stored at −80° C.

TABLE 3
Stability of purified variants.

| | | Freeze/Thaw 3 cycles | | | |
|---|---|---|---|---|---|
| | | | % CoV-2 | | |
| | | % homo- | S-RBD | Folding stability | |
| | SEQ | dimer | binding | Tₘ^{ACE2} | Tₘ^{CH3} |
| | ID | (UPLC- | similarity | (° C.) | (° C.) |
| Variant name | NO | SEC) | (SPR) | (DSC) | (DSC) |
| ACE2-SG4-Fc | 15 | 99.3 | 100 | 52.3 | 83.1 |
| ACE2-SG4x3-Fc | 17 | 99.5 | 99 | 52.5 | 82.9 |
| ACE2m1-SG4-Fc | 18 | 98.6 | 100 | 49.8 | 83.1 |
| ACE2m2-SG4-Fc | 19 | 99.1 | 98 | 52.2 | 83.0 |
| ACE2m3-SG4-Fc | 20 | 99.2 | 99 | 50.3 | 83.1 |
| ACE2m4-SG4-Fc | 21 | 99.1 | 99 | 50.0 | 82.6 |
| ACE2m4-C261S- | 22 | 96.5 | 100 | 50.5 | 83.3 |
| SG4-Fc | | | | | |
| ACE2m4-hinge2CS- | 23 | 98.9 | 100 | 50.3 | 82.5 |
| SG4-Fc | | | | | |

### Folding Stability

We were also interested to find out if there are differences in the stability of various structured domains of the polypeptide constructs of this class. To this end, differential scanning calorimetry (DSC) was used to determine thermal transition midpoints (Tₘ) of the polypeptide constructs exemplified in this invention. DSC experiments were performed using a VP-Capillary DSC system (Malvern Instruments Ltd, Malvern, UK). Samples were diluted in HyClone™ Dulbecco's phosphate-buffered saline (DPBS; GE Healthcare Life Sciences) to a final concentration of 0.4 mg/mL. Thermal denaturation was carried out under 70 psi of nitrogen pressure by increasing the temperature from 20° C. to 100° C. at a rate of 60° C./h, with feedback mode/gain set at “low”, filtering period of 8 s, prescan time of 3 min. All data were analyzed with Origin 7.0 software (OriginLab Corporation, Northampton, MA). Thermograms were corrected by subtraction of corresponding buffer blank scans and normalized to the protein molar concentration. Tₘ values were determined using automated data processing with the rectangular peak finder algorithm for Tₘ. Data listed in Table 3 indicate that all variants have transitions at both ˜50° C. and ˜82° C. of approximately equal enthalpy, implying two common features. The two common structured features of these compounds are the ACE2 catalytic domain (R1 region) and the C_{H}2-C_{H}3 domains (R4 region). The C_{H}3 domain of the antibody IgG1 Fc fragment is known to have a melting temperature at ˜82° C. The ACE2 catalytic domain accounts for the transitions around 50° C. [39]. A structure-stability relationship analysis based on the Tₘ data from Table 3 suggests that the N330Y mutation present in certain variants is responsible for lowering the Tₘ of the ACE2 catalytic domain by about 2° C.

### Example 5. Enzymatic Activity of Polypeptide Constructs

### Catalytic Efficiency

The enzymatic activities of the polypeptide constructs of this invention were assayed with the fluorogenic substrate Mca-APK(Dnp) (AnaSpec, San Jose, CA, Cat #: AS-60757) as described previously [40]. Recombinant human ACE2 (rhACE2) was used as positive control (R&D Systems Inc., Minneapolis, MN, Cat #: 933-ZN) and blank as negative control. The assay buffer contained 50 mM 2-(N-Morpholino)ethanesulfonic acid (MES), 300 mM NaCl, 10 μM ZnCl₂, pH 15 to 6.8. Variants and rhACE2 control were tested at 100 ng/mL concentration, and substrate concentrations were 1, 2, 4, 6, 8 and 16 μM. Samples were assayed in triplicate with or without 10⁵ M ACE2 inhibitor MLN-4760 (Calbiochem, Cat #: 530616). Reactions were followed kinetically with readings every 62 s up to 32 min on a FLUOstar Galaxy fluorometer (BMG Labtechnologies, Durham, NC, USA), detecting emission at 405 nm with excitation at 320 nm. Data were fitted and plotted using Grafit (Sigma-Aldrich). Relative fluorescence units (RFU) were subtracted for samples with MLN-4760 and converted into concentration units (M) of product based on the standard curve of product formation. For calculation of catalytic efficiencies, k_{cat}/K_{M}, initial velocities (V₀) determined from the linear phase of product formation over time were plotted against substrate concentration. Using the linear phase of the V₀ versus substrate concentration plots, k_{cat}/K_{M} values were calculated by dividing V₀ by substrate concentration at substrate concentration below 6 μM. [We gratefully acknowledge that some of the technical data relating to the catalytic efficiency assays were kindly provided by the lab of Dr. Kevin Burns (OHRI, University of Ottawa, Ottawa, Canada)].

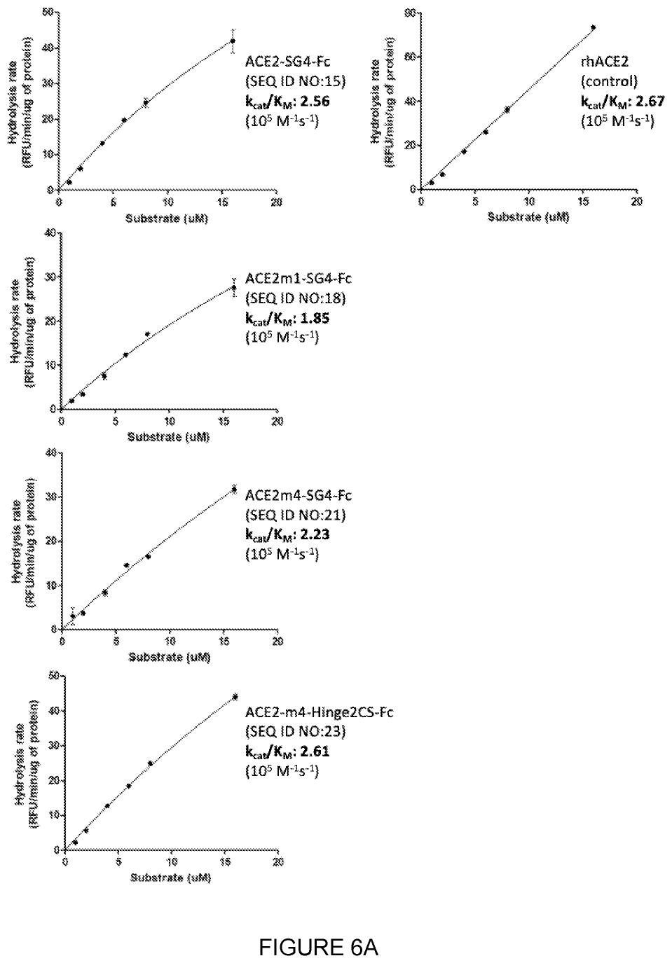

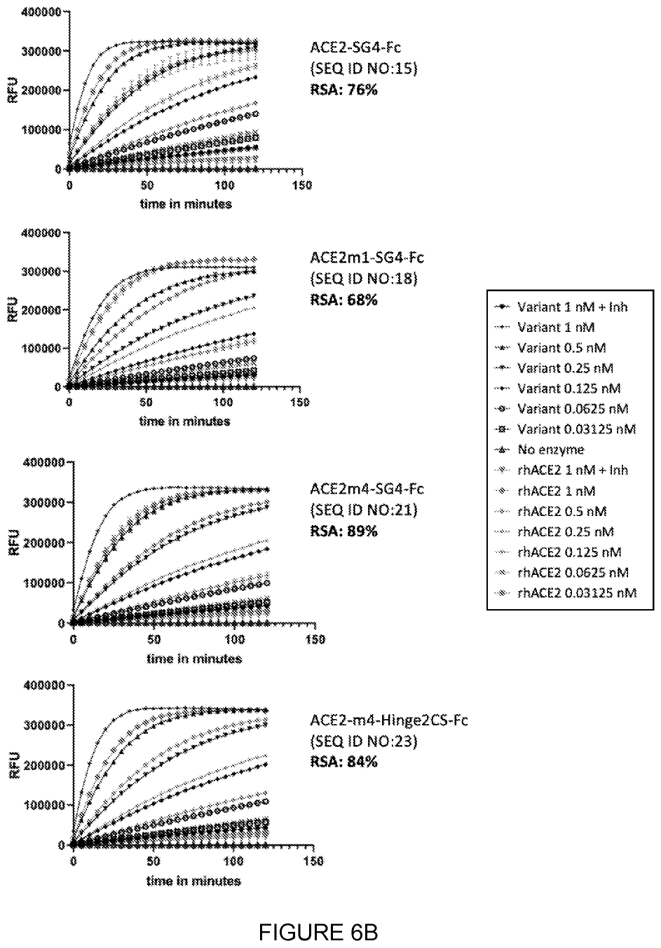

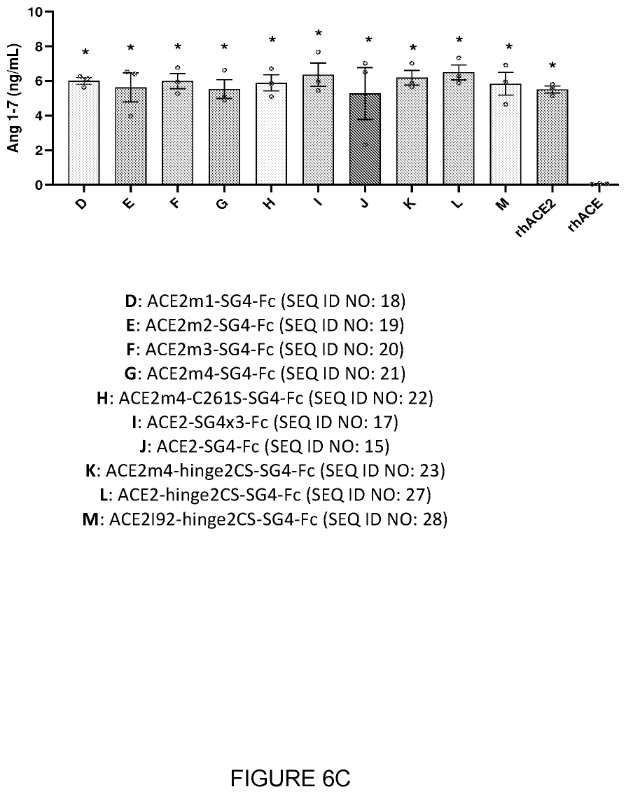

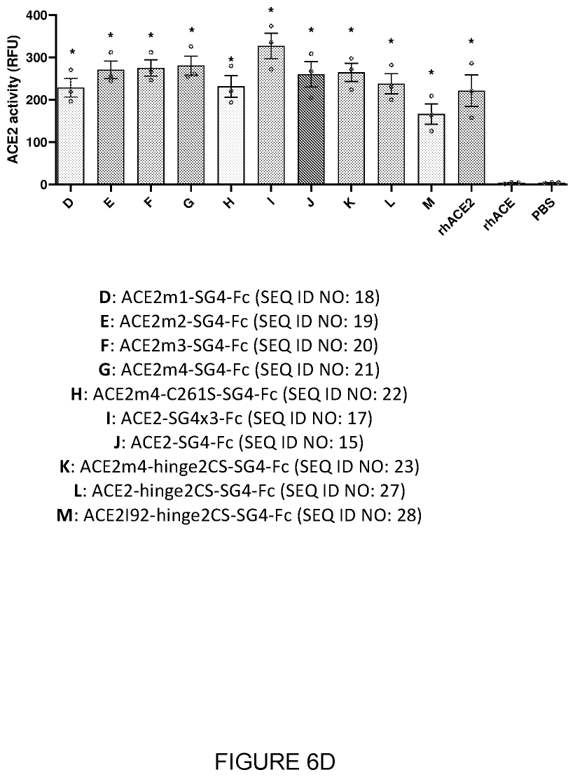

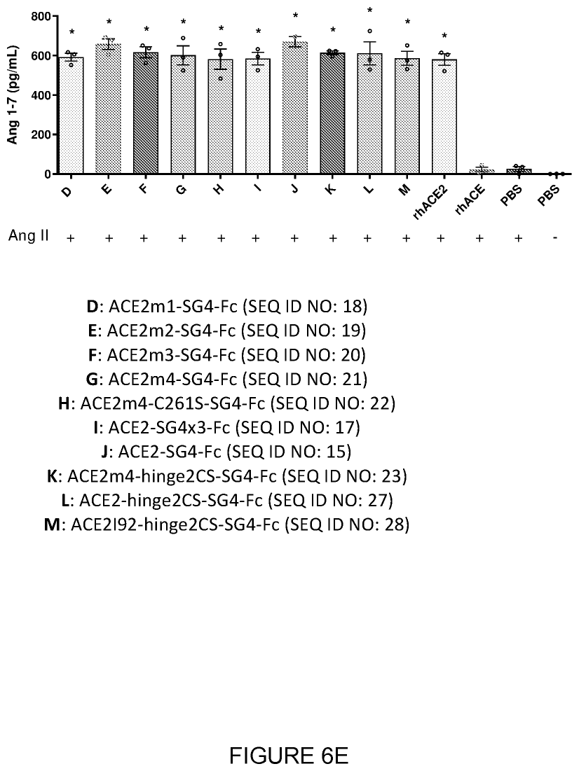

Catalytic efficiencies (k_{cat}/K_{M}) are listed for all tested polypeptide constructs in Table 4, and the V₀ versus substrate concentration plots are shown for the rhACE2 positive control and selected variants in FIG. 6A. As it can be seen from these data, all polypeptide constructs of this invention have high catalytic efficiencies which are in the same order of magnitude (10⁵ M⁻¹s⁻¹) with rhACE2, which has a k_{cat}/K_{M} of 2.67×10⁵ M⁻¹s⁻¹ as determined in this study. This indicates that structural alterations introduced during molecular design and optimization as described in Example 1 do not have a major impact on catalytic activity. Moreover, a structure-activity relationship analysis of the enzymatic activity data reveals the important and unpredicted role of T92I natural mutation in increasing ACE2 enzymatic activity in our novel constructs. This effect is best highlighted by comparing the variant ACE2m1-SG4-Fc (SEQ ID NO:18) having a Thr at position 92 with ACE2m4-SG4-Fc (SEQ ID NO:21) having an Ile at position 92, the only difference between these variants being this natural mutation and the consequential loss of carbohydrate structure linked at Asn90. Further support for this finding is provided by variant ACE2m4-hinge2CS-SG4-Fc (SEQ ID NO:23) that has exactly the same ACE2 catalytic domain (R1 region) as ACE2m4-SG4-Fc (SEQ ID NO:21). The higher catalytic efficiencies of ACE2m4-SG4-Fc (SEQ ID NO:21) and ACE2m4-hinge2CS-SG4-Fc (SEQ ID NO:23) relative to ACE2m1-SG4-Fc (SEQ ID NO:18) are immediately apparent from FIG. 6A and Table 4.

### Specific Activity

We also characterized the specific activities of the polypeptide constructs and compered them with the specific activity of the rhACE2 positive control. Specific activities were determined using the Angiotensin Converting Enzyme (ACE2) Fluorometric Activity Assay kit (BioVision, Milpitas, CA, Cat #: K897-100). We replaced the provided enzyme in the kit (concentration not disclosed) by the commercial rhACE2 used in the catalytic efficiency study described above. The manufacturer protocol was followed. Assays were conducted in a 96-well format in duplicate. Concentrations of all polypeptide constructs ranged from 1 nM to 0.03125 nM in a 2-fold serial dilution. Reactions were followed kinetically on a Cytation 5 instrument (BioTek) with excitation at 320 nm and emission at 420 nm. For calculation of specific activities, two time points in the linear range of the activity time-course plot were chosen at enzyme concentrations of 0.25 nM (time points 20 and 25 min) and 1 nM (time points 5 and 10 min) which correspond to linear phases of the plot (FIG. 4B). Duplicate values for each time point were averaged and normalized to zero based on blank data. Specific activity (pmol/min/mg) was then calculated by dividing the change in RFU between the time points by the corresponding time range and the amount of enzyme used. Final specific activity data reported in Table 4 is the average between data at the two enzyme concentrations (0.25 nM and 1 nM) normalized to the specific activity of the rhACE2 control on each plate.

It is readily apparent that the polypeptide constructs of this invention have high specific activities in the order of 10⁶ pmol(product)/min/mg(enzyme) (FIG. 6B and Table 4), and are in the 68-89% range relative to the rhACE2 control. In agreement with the catalytic efficiency data presented in the previous section, the mutations at positions 27 and 330 of the ACE2 catalytic domain (R1 region) slightly decrease enzymatic activity, as seen with the variant ACE2m1-SG4-Fc (SEQ ID NO:18). However, the specific activity is restored by adding the including the naturally occurring mutation to lie at position 92 with removal of N-linked carbohydrate at position 90 of the ACE2 catalytic domain—compare ACE2m4-SG4-Fc (SEQ ID NO:21) and ACE2m4-hinge2CS-SG4-Fc (SEQ ID NO:23) variants relative to ACE2m1-SG4-Fc (SEQ ID NO:18).

Taken together, the catalytic efficiency and specific activity data of the polypeptide constructs of this invention suggest that these compounds will be effective in restoring the protective effects of the alternative renin-angiotensin system by efficiently converting Ang-II to Ang(1-7) and thus act as therapeutic agents for reducing the severity of ARDS.

TABLE 4
Enzymatic activity characterization using
on vitro cell-free fluorogenic assays.

| | | | | | Normalized |
|---|---|---|---|---|---|
| | | | Relative | Specific | relative |
| | | | k_{cat}/K_{M} | activity | specific |
| | SEQ | k_{cat}/K_{M} | (% | (×10⁶ | activity (% |
| | ID | (×10⁵ | rhACE2 | pmol/ | rhACE2 |
| Variant | NO | M⁻¹s⁻¹) | control) | min/mg) | control) |
| ACE2-SG4-Fc | 15 | 2.56 | 95.9 | 4.91 | 76 |
| ACE2-SG4x3-Fc | 17 | 1.57 | 58.8 | 4.81 | 75 |
| ACE2m1-SG4-Fc | 18 | 1.85 | 69.3 | 4.40 | 68 |
| ACE2m2-SG4-Fc | 19 | 0.91 | 34.1 | 5.44 | 85 |
| ACE2m3-SG4-Fc | 20 | 1.42 | 53.2 | 5.22 | 81 |
| ACE2m4-SG4-Fc | 21 | 2.23 | 83.5 | 5.71 | 89 |
| ACE2m4-C261S- | 22 | 1.37 | 51.3 | 4.98 | 77 |
| SG4-Fc | | | | | |
| ACE2m4- | 23 | 2.61 | 97.8 | 5.43 | 84 |
| hinge2CS-SG4-Fc | | | | | |
| ACE2- | 27 | 3.06 | 114.6 | na | na |
| hinge2CS-SG4-Fc | | | | | |
| ACE2l92- | 28 | 2.94 | 110.1 | na | na |
| hinge2CS-SG4-Fc | | | | | |

na: not assessed.

### Angiotensin II Hydrolytic Activity

ACE2 enzymatic activity was also determined directly, by incubation with angiotensin II (Ang II), followed by measure of angiotensin-(1-7) (BMA biomedicals, Cat #: S-1330). Select polypeptides of this invention or control enzymes (rhACE2, rhACE) at 0.5 μg/mL were added to assay buffer containing 25 nM Ang II (R&D systems, Cat #: 1158/5), with or without 10-M MLN-4760 for 30 min at room temperature. FIG. 6C shows that the Ang II hydrolytic activities of all tested polypeptides of this invention are significantly higher than the negative control, human recombinant ACE (rhACE) and comparable to the positive control, the commercial recombinant human ACE2 (rhACE2). [We gratefully acknowledge that some of the technical data relating to angiotensin II hydrolytic activity were kindly provided by the lab of Dr. Kevin Burns (OHRI, University of Ottawa, Ottawa, Canada)].

### Enzymatic Activity and Stability in Primary Cultures of Mouse Proximal Tubular Epithelial Cells

Proximal tubular epithelial cells (PTECs) were isolated from C57BL/6 male and female mice, age 12-16 weeks, obtained from Charles Rivers (Saint-Constant, Quebec, Canada). Isolation was performed as previously described [41]. For each isolation, kidneys from 1 male and 1 female mouse were collected in cold perfusion solution [containing (in mM) 1.5 CaCl₂, 5.0 D-glucose, 1.0 MgSO₄, 24 NaHCO₃, 105 NaCl, 4.0 Na lactate, 2.0 Na₂HPO₄, 5.0 KCl, 1.0 L-alanine, 10 N-2-hydroxyethylpiperazine-N-2-ethanesulfonic acid (HEPES), and 0.2% bovine serum albumin (BSA)]. Kidney cortices were minced and digested in perfusion solution with the addition of 0.1% collagenase (Sigma-Aldrich, Cat #: C9262) and 0.05% soybean trypsin inhibitor (Sigma-Aldrich, Cat #: T6522), pH 7.2. The cortical digestion was passed through a 250 μm sieve, pelleted, and resuspended in 40% Percoll (Sigma-Aldrich, Cat #: P1644) solution containing (in mM) 5.0 D-glucose, 10 HEPES, 1.0 MgCl₂, 120 NaCl, 4.8 KCl, 25 NaHCO₃, 1.0 NaH₂PO₄, 1.0 L-alanine, 1.4 CaCl₂, 60 U/mL penicillin, and 60 μg/mL streptomycin. The digested product was centrifuged at 18,500×g for 30 min. After centrifugation, the 4^{th} layer containing PT cells was aspirated, pelleted, and resuspended in culture media (DMEM/F12—Gibco, Cat #: 31600034 and 21700075). Cells were seeded onto 24-well plates and cultured for 24 h in DMEM/F12 (1:1) medium containing 10% fetal bovine serum (FBS) and defined medium (5 μg/mL insulin transferrin sodium selenate, 50 nM hydrocortisone, 2 nM 3,3′,5-triiodo-L-thyronine (Sigma Aldrich Cat #: 11884, H0888, T5516), 100 U/mL penicillin, and 100 mg/mL streptomycin. After 24 h, cells were grown in DMEM/F12 and defined medium. On day 6 (at ˜70% confluency), PTECs were treated with 0.6 nM of rhACE2, polypeptides of this invention, rhACE (negative control), or phosphate buffered saline (PBS, control) for 24 h. Following treatment with variants, either angiotensin II or Mca-APK(Dnp) was added to each well, to a final concentration of 5 nM Ang II or 11.25 μM Mca-APK(Dnp). For PTECs treated with Ang II, media was collected and assayed for angiotensin-(1-7) by ELISA (BMA Biomedicals, Cat #: S-1330). PTECs incubated with fluorescent substrate had 100 μL removed from each well, transferred to a black 96-well plate, and fluorescence was determined as described for ACE2 enzymatic activity.

To test the activity and stability of ACE2-Fc variants in vitro, as described above, cultured primary mouse proximal tubular cells were treated with select polypeptides of this invention, enzyme controls (rhACE2, rhACE) or PBS for 24 h, followed by incubation with either fluorogenic substrate (FIG. 6D) or Ang II (FIG. 6E). All tested polypeptides of this invention remained active after 24 h at 37° C., and their activities with either the fluorogenic substrate (FIG. 6D) or Ang II (FIG. 6E) were significantly higher than the negative control (rhACE) and comparable to that of the negative control (rhACE2). [We gratefully acknowledge that some of the technical data relating to enzymatic activity in primary cell cultures were kindly provided by the lab of Dr. Kevin Burns (OHRI, University of Ottawa, Ottawa, Canada)].

### Example 6. Binding of Polypeptide Constructs to SARS-CoV-2 Spike Protein

Dissociation Rates from Spike-RBD by SPR

Binding kinetics of the polypeptides of this invention to the receptor binding domain (RBD) of SARS-CoV-2 spike protein were carried out by surface plasmon resonance (SPR) experiments on a Biacore T200 instrument (GE Healthcare) at 25° C. in a PBST pH 7.4 running buffer (PBS with 0.05% Tween 20 and 3.4 mM EDTA). In-house produced [42] SARS-CoV-2 S-RBD Wuhan and B.1.315 variant and SARS-CoV-1 S-RBD were immobilized via amine coupling on a CM-5 sensorchip at 200 RUs. Single cycle kinetics were determined by injecting each polypeptide construct variant at 3-fold serial dilutions (top nominal concentrations of 30, 60 or 120 nM) and a buffer blank was simultaneously injected over the blank and S-RBD surfaces at 50 μL/min for 120 s with a 900 s dissociation phase. Regeneration was done for 30 s with 10 mM glycine pH 1.5 at a flow rate of 30 μL/min. Data analysis was done with the Biacore T200 Evaluation software. Double referenced sensorgrams were aligned to the baseline and normalized to the end of the 60 nM injection for off-rate ranking. Off-rates, k_{off} (M⁻¹s⁻¹), were determined from the 1:1 fit of the double-referenced data and tabulated Table 5. Avidity effects due to bivalent nature of the flowed polypeptide constructs and the density of the immobilized S-RBD prevented determination of thermodynamic equilibrium constants, K_{D}.

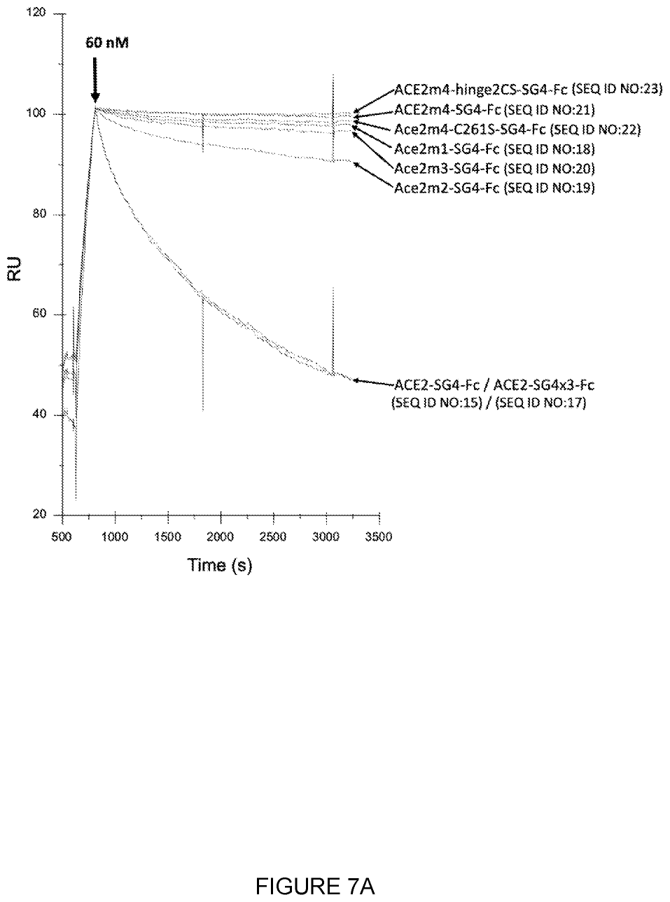

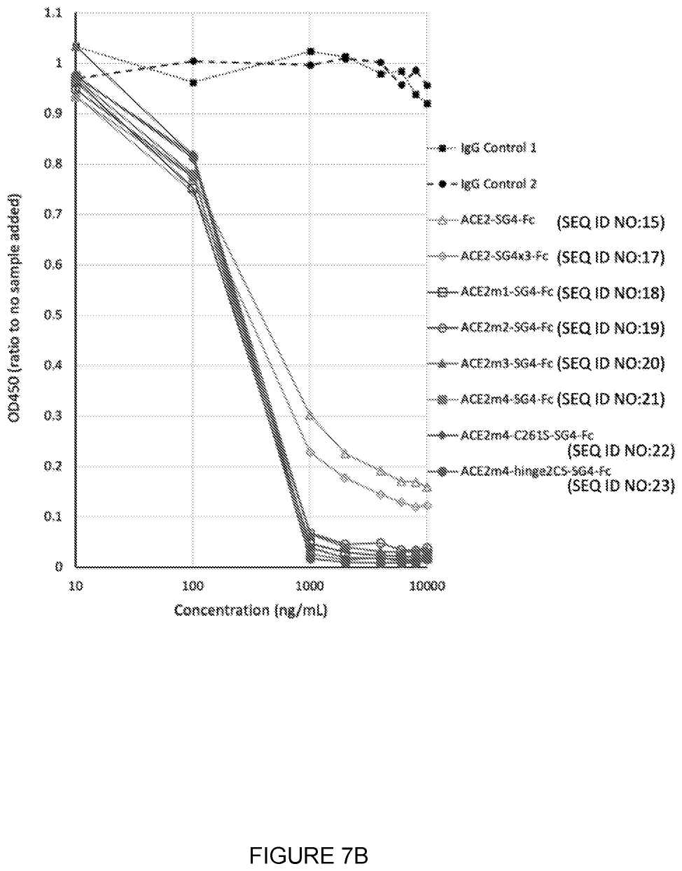

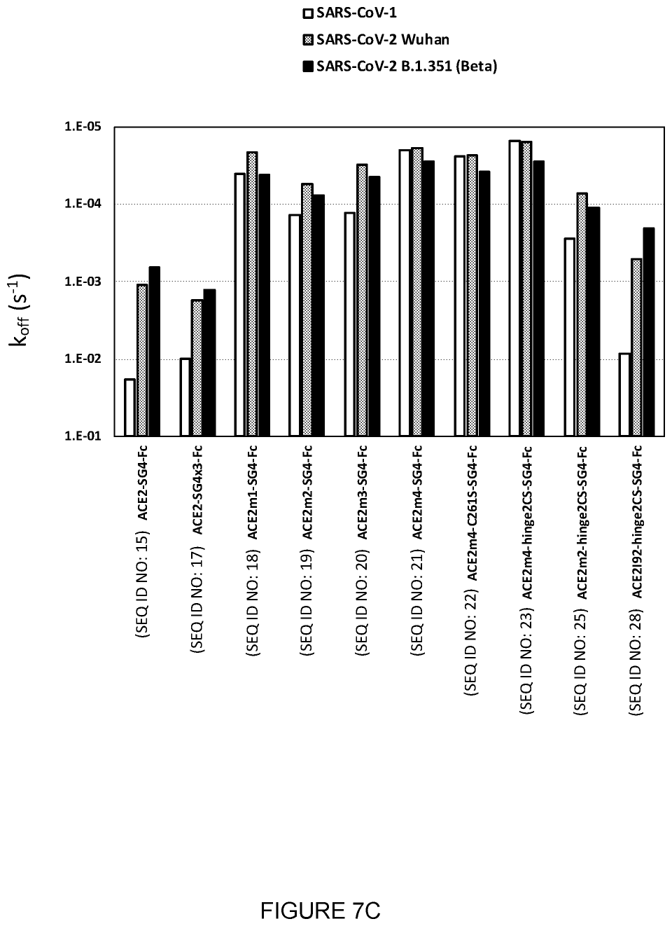

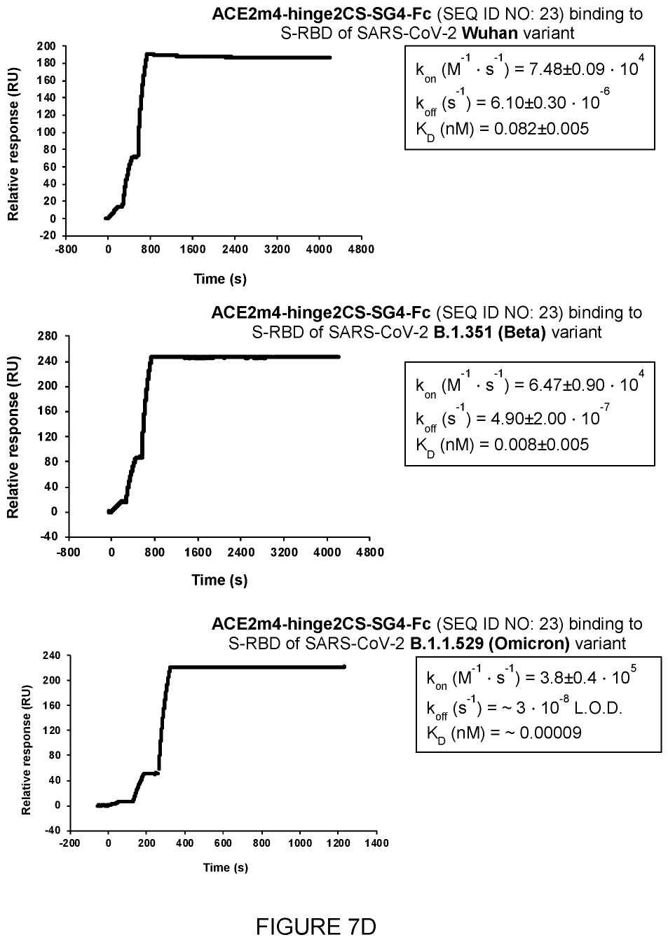

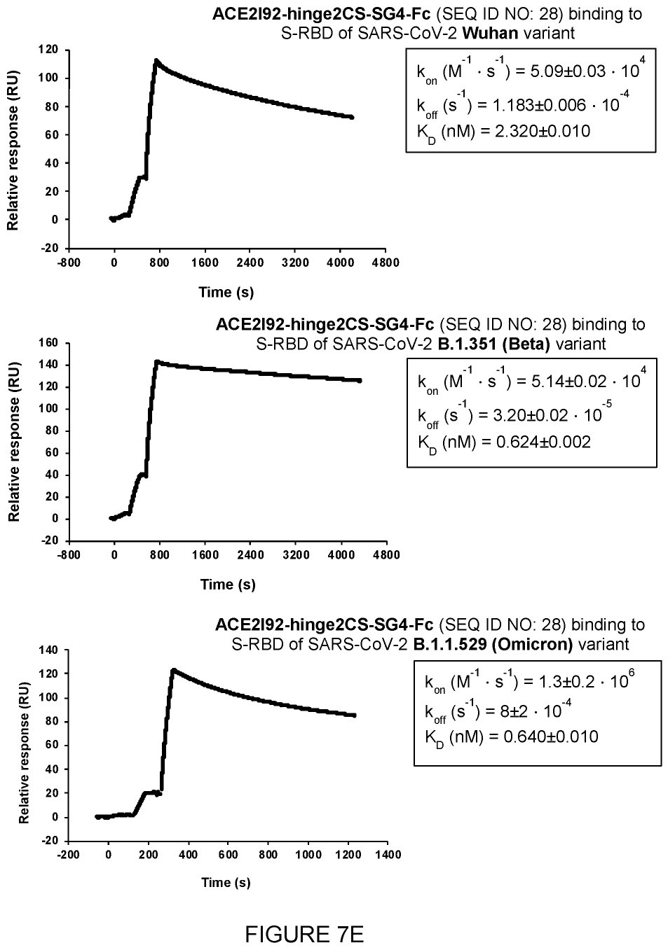

The aligned normalized sensorgrams obtained for the tested polypeptide constructs of this invention are shown in FIG. 7A and are useful in ranking the dissociation rates of these compounds from SARS-CoV-2 RBD. Dissociation rates are typically regarded as the main criterion in selecting the best binders for further therapeutic development. It is immediately apparent from FIG. 7A that ADAPT affinity changes at positions 27 and 330 along with the natural mutation at position 92 of the ACE2 catalytic domain (R1 region) lead to significantly decreased dissociation rates thus reflecting increased interactions with the SARS-CoV-2 domain relative to variants with unoptimized ACE2 catalytic domain: ACE2-SG4-Fc (SEQ ID NO:15) and ACE2-SG4x3-Fc (SEQ ID NO:17). The slowest dissociation rates are observed for the three ACE2m4 variants (SEQ ID NOs:21, 22 and 23), followed closely by ACE2m1-SG4-Fc (SEQ ID NO:18) and ACE2m3-SG4-Fc (SEQ ID NO:20) variants, and then ACE2m2-SG4-Fc (SEQ ID NO:19) which has a faster k_{off} that is still much slower than for the unoptimized ACE2 variants. Structure-activity relationship of dissociation rates in Table 5 point to a larger role of the N330Y substitution to improving binding interactions to SARS-CoV2 RBD than that of the T27Y substitution. The slowest dissociation rates obtained in this series of polypeptide constructs is in the order of 10⁻⁶ s⁻¹, indicating very tight binding.

Binding Competition for Spike-RBD by snELISA

A unique and clinically-relevant surrogate neutralization (sn) ELISA-based assay [43] was also used as an additional method for in vitro testing of binding capacity of the polypeptide constructs of this invention to SARS-CoV-2 spike protein. In this assay, the spike-RBD was immobilized on multi-well plates and then incubated with the sample being tested for neutralization potential. Biotinylated hACE2 is then added with detection by streptavidin-polyHRP. The ability of the sample to block the interaction of biotinylated hACE2 and the antigen can be measured by a dose-dependent decrease in signal. We used 100 ng immobilized recombinant RBD on 96-well Immulon HBX plates incubated overnight at 4° C. (2 μg/mL). All volumes added to the well were 50 μL. Plates were washed 3 times with 200 μL PBS-T and blocked for 1-1.5 hours at room temperature with 200 μL 3% BSA (BioShop Canada Inc., SK1400.1). After washing as above, a 4-step, 2-fold serial dilution series of patient serum or plasma (0.5-4 μL of sample) was incubated for 1 h. The wells were washed as above and incubated with 50 ng biotinylated recombinant human ACE2 for 1 h. After washing as above, the wells were incubated with 44 ng streptavidin-peroxidase polymer (MilliporeSigma, S2438). The resultant signal was developed and quantified with TMB. Due to day-to-day variation in signal, all OD₄₅₀ values are normalized to the OD₄₅₀ of the well where no sample was added for each sample. All values are expressed in this ratio space.

The IC90 values obtained with the snELISA assay are listed in Table 5 and dose-response curves are shown in FIG. 7B. There is a qualitative agreement between the snELISA IC90 values and the k_{off} dissociation rates from the SPR binding experiment. The snELISA experiment hereby further confirms that the structural optimization performed in the ACE2 catalytic domain of the polypeptide constructs of this invention significantly improve the binding characteristics to SARS-CoV-2 spike-RBD, relative to the unoptimized variants. An important aspect that is apparent from FIG. 7B is that these compounds are capable of reducing binding to biotinylated ACE2 used as competitor in the assay to very low levels at the higher doses. This strongly suggest that these compounds are capable of completely neutralizing the SARS-CoV-2 virus and prevent its attachment to the ACE2 on host cells. This strongly indicates that these compounds can be used a potent antiviral agents.

TABLE 5
Binding to SARS-CoV-2 Wuhan spike protein RBD.

| | | k_{off} | IC90 |
|---|---|---|---|
| | SEQ | (×10⁻⁶s⁻¹) | (ng/mL) |
| Variant | ID NO | (SPR) | (snELISA) |
| ACE2-SG4-Fc | 15 | 667 | >2000 |
| ACE2-SG4x3-Fc | 17 | 577 | >2000 |
| ACE2m1-SG4-Fc | 18 | 15 | 225 |
| ACE2m2-SG4-Fc | 19 | 34 | 300 |
| ACE2m3-SG4-Fc | 20 | 19 | 225 |
| ACE2m4-SG4-Fc | 21 | 3 | 150 |
| ACE2m4-C261S-SG4-Fc | 22 | 9 | 150 |
| ACE2m4-hinge2CS-SG4-Fc | 23 | 3 | 150 |

### Binding to SARS-CoV Variants

In addition to evaluating the dissociation rates from the spike-RBD of the original Wuhan SARS-CoV-2, a larger number of polypeptides of this invention were also evaluated for the ability to bind to the immobilized S-RBD of the B.1.351 (Beta) variant of concern of SARS-CoV-2, as well as to the S-RBD of the earlier SARS-CoV-1, which is more distant phylogenetically from the Wuhan SARS-CoV-2 [44]. As shown in the graph displayed in FIG. 7C, all tested polypeptides of this invention displayed dissociation rates from the S-RBD of the B.1.351 (Beta) variant which were similar to those from the S-RBD of the original Wuhan virus variant. Noteworthy, the polypeptide ACE2I92-hinge2CS-SG4-Fc (SEQ ID NO: 28) of this invention, in this assay, had an even slower dissociation rate from the S-RBD of the B.1.351 (Beta) variant than from S-RBD of the original Wuhan virus.

For select variants, in order to derive more precise estimates of kinetic constants and an apparent K_{D} with avidity, replicated measurements were carried out by immobilizing purified spike-RBD at approximately 500 RUs and injecting each polypeptide construct at 3, 30 and 300 nM over the blank and S-RBD surfaces at 50 μL/min for 180 s with a longer dissociation phase of 3600 s. FIG. 7D shows the SPR sensorgrams for the polypeptide ACE2m4-hinge2CS-SG4-Fc (SEQ ID NO: 23) of this invention to the S-RBS of Wuhan, B.1.351 (Beta) and B.1.1.529 (Omicron) variants of SARS-CoV-2, and FIG. 7E shows the SPR sensorgrams for the polypeptide ACE2I92-hinge2CS-SG4-Fc (SEQ ID NO: 28) of this invention to the S-RBS of Wuhan, B.1.351 (Beta) and B.1.1.529 (Omicron) variants of SARS-CoV-2. The polypeptide ACE2m4-hinge2CS-SG4-Fc (SEQ ID NO: 23) binds with very slow kinetic dissociation rates (k_{off} around 10⁻⁶ M⁻¹s⁻¹) and picomolar dissociation equilibrium constants (K_{D}) to the S-RBD of either of these two important SARS-CoV-2 variants, with binding apparently being 10-fold stronger to the B.1.135 variant. However, the SPR experiment has reached the limit of detection (L.O.D.) for these protein complexes due to very low dissociation rates. We also carried out SPR binding experiments of the polypeptide ACE2m4-hinge2CS-SG4-Fc (SEQ ID NO: 23) to the B.1.1.529 (Omicron) spike protein RBD variant, and observed the same behavior, i.e., extremely strong binding that reached the L.O.D. of the SPR method. The polypeptide ACE2I92-hinge2CS-SG4-Fc (SEQ ID NO:28) binds with slow kinetic dissociation rates (k_{off} around 10⁻⁴ M⁻¹s⁻¹) and nanomolar dissociation equilibrium constants (K_{D}) to the S-RBD of either of these important SARS-CoV-2 variants. Binding was stronger (4-fold) to the B.1.135 virus variant than to the original Wuhan virus. We also carried out SPR binding experiments of the polypeptide ACE2I92-hinge2CS-SG4-Fc (SEQ ID NO:28) to the B.1.1.529 (Omicron) S RBD variant, and observed the same behavior, i.e., about 4-fold improved binding affinity relative to the original Wuhan virus spike protein RBD. Taken together, these data clearly demonstrates the broad pan-specificity of the polypeptides of this invention to effectively bind to all SARS-CoV-2 variants currently in circulation and, with very high probability, to other mutated SARS-CoV-2 variants that may emerge in the future.

### Example 7. SARS-CoV-2 Pseudovirus Neutralization by Polypeptide Constructs

### Pseudotyped VLP Assay

The virus-like particle (VLP) based spike pseudotype viral surrogate assay was used to evaluate the polypeptide constructs of this invention for inhibition of SARS-CoV-2 virus entry into the host cell mediated by binding of viral spike protein to the human ACE2 receptor present at the host cell surface. This assay uses a pseudotyped lentiviral particle as a substitute for the live SARS-CoV-2 virus and measures the ability of compounds of interest to block entry of the VLP into a host cell line expressing ACE2 [45]. The pseudotyped VLP contains the SARS-CoV-2 spike protein, a luciferase reporter and the minimal set of lentiviral proteins required to assemble the VLPs. Blockage of viral entry is detected by loss of signal of the luciferase reporter. Two slightly different pseudotyped VLP assay implementations, named Method 1 and Method 2, were used to evaluate the polypeptides of this invention.

In Method 1, the assay that was initially developed by the Bloom lab [45] was optimized to increase robustness and reproducibility [43]. The main changes were: 1) co-expressing TMPRSS2 with ACE2 in the HEK293T cells that improves the efficiency of infection; 2) using a 2^{nd} generation lentivirus packaging system (which yielded higher VLP levels); 3) adapting the VLP production conditions, including decreasing the temperature to 33° C. for VLP production, which improved the consistency in the quality of VLPs produced. Entry vectors for ACE2 and TMPRSS2 coding sequences were cloned into pLenti CMV Puro DEST (Addgene, 17452) and pLenti CMV Hygro DEST (Addgene, 17454), respectively. The resulting transfer vectors were used to generate lentivirus via the second-generation psPAX2 and VSV-G (Addgene, 8454). HEK293T cells were transduced with ACE2 lentivirus at an MOI<1 and selected with puromycin (1 μg/mL) to generate a stable population. These cells were subsequently transduced with TMPRSS2 lentivirus and selected with hygromycin (200 μg/mL) in a similar fashion. For VLP generation, HEK293T cells were transiently cotransfected in a 6-well-plate format containing 2 mL growth medium (10% FBS, 1% penicillin/streptomycin [Pen/Strep] in DMEM) with 1.3 μg psPAX2,1.3 μg pHAGE-CMV-Luc2-IRES-ZsGreen-W (BEI, NR-52516; lentiviral backbone plasmid that uses a CMV promoter to express luciferase followed by an IRES and ZsGreen), and 0.4 μg HDM-IDTSpike-fixK (BEI, NR-52514; expressed under a CMV promoter a codon-optimized Wuhan-Hu-1 spike; GenBank, NC_045512) using 8 μL JetPrime (Polyplus-transfection SA, 114-01) in 500 μL JetPrime buffer. After 8 h of transfection, the medium was replaced by 3 mL of DMEM containing 5% heat-inactivated FBS and 1% Pen/Strep, and the cells were incubated for 16 h at 37° C. and 5% CO2; they were then transferred to 33° C. and 5% CO2 for an additional 24 h. At 48 h after transfection, the supernatant was collected, spun at 500×g for 5 min at room temperature, filtered through a 0.45 μm filter and frozen at −80° C. The virus titers were evaluated using HEK293T-ACE2/TMPRSS2 cells at 10,000 cells per well on a Poly-L-Lysine-coated (5-10 μg/mL) 96-well plate using HI10 media (10% heat-inactivated FBS, 1% Pen/Strep), along with a virus dilution resulting in >1000 relative luciferase units (RLU) over control (˜1:100 virus stock dilution). For the neutralization assay, 2.5-fold serial dilutions of the serum samples were incubated with diluted virus at a 1:1 ratio for 1 hour at 37° C. before being transferred to plated HEK293-ACE2/TMPRSS2 cells and incubated for an additional 48 h at 37° C. and 5% CO2. After 48 h, cells were lysed, and Bright-Glo luciferase reagent (Promega, E2620) was added for 2 minutes before reading with a PerkinElmer Envision instrument. 50% inhibitory concentration or dilution (IC50 or ID50) were calculated with nonlinear regression (log[inhibitor] versus normalized response−variable slope) using GraphPad Prism 8 (GraphPad Software Inc.).

Method 2 is also based also based on the same published protocol [45] and is thus conceptually similar with Method 1, with a few notable differences including among others: (i) no co-expression of TMPRSS2 on the HEK293T cell line expressing human ACE2, and (ii) lentivirus VLP production at 37° C. by transient transfection of HEK293SF cells.

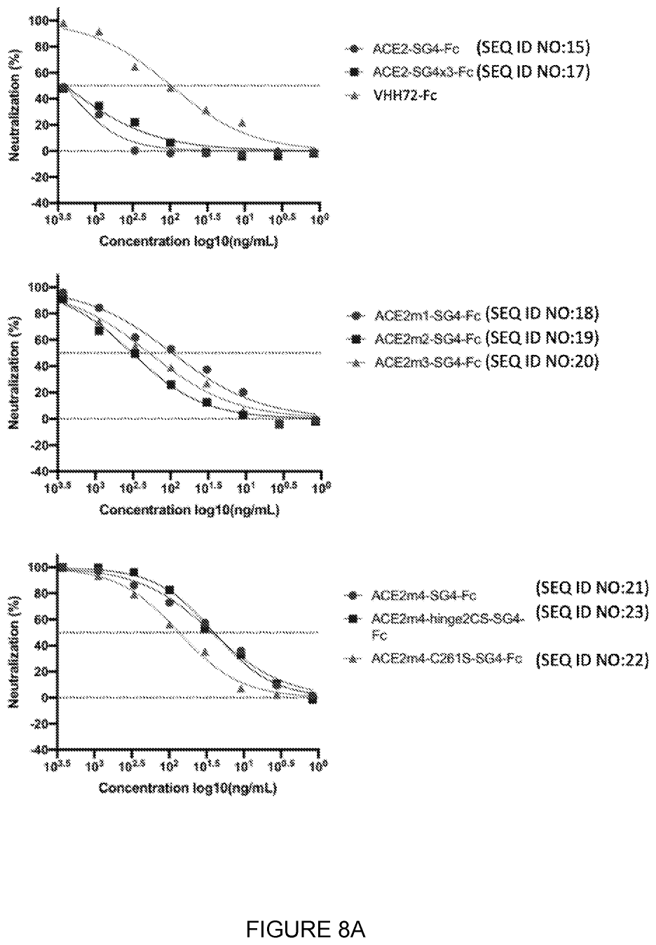

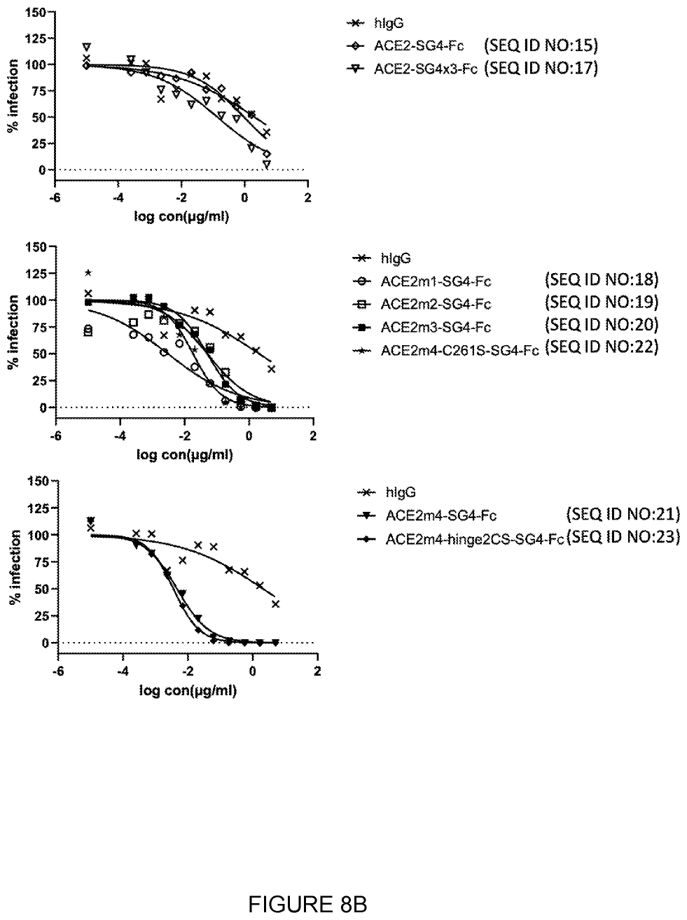

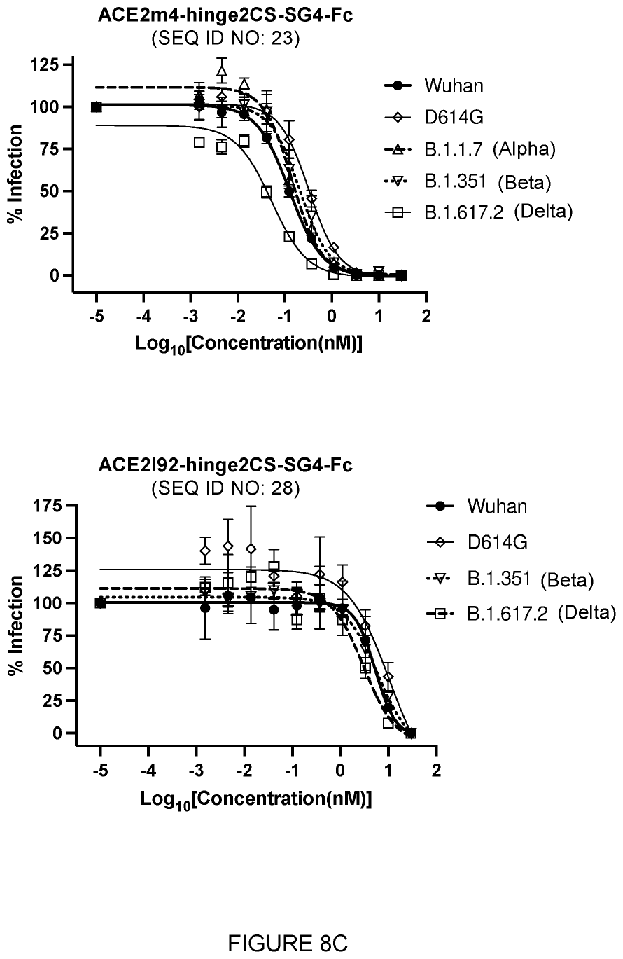

The IC50 data obtained with both methods for the polypeptide constructs of this invention are listed in Table 6, whereas the dose-response curves are shown in FIG. 8. First, we note the excellent correlation between the IC50 values obtained with the two implementations, with a linear correlation R² of 1.0 between the IC50 values and R² of 0.99 between the log₁₀(IC50) values. Furthermore, these pseudovirus neutralization data correlate almost perfectly with the spike dissociation rates from the spike-RBD obtained by SPR binding experiment as described earlier and listed in Table 5, with R²=0.99 between k_{off} and IC50 (either pseudovirus neutralization method), and with R² values of 0.96-0.99 between log₁₀k_{off} and log₁₀IC50 (either pseudovirus neutralization method). These extremely high correlations indicate the robustness of the data and firmly confirm the mechanism of action of the polypeptide constructs of this invention, i.e., binding to viral spike-RBD prevents virus entry into host cell. We also note that while there is an excellent correlation between the IC50 data obtained with the two methods, the absolute IC50s are translated to higher values with Method 1 relative to Method 2. This effect can be due to the different implementations, and particularly to including co-expression of TMPRSS2 on the ACE2 expressing cells with Method 1 but not with Method 2. Co-expression of TMPRSS2 may facilitate viral entry and infection, and hence explain why neutralization occurs at higher doses of the compounds with Method 1 relative to Method 2 that does not include TMPRSS2.

The best pseudo-virus neutralization levels achieved are excellent with both methods, with some of the ACE2m4 variants (SEQ ID NOS:21,23) reaching IC50 values of ˜25 ng/mL with Method 1 and 3 ng/mL with Method 2 (Table 6). It is immediately apparent that the sequence and structure optimizations carried out during the polypeptide molecular engineering phase (see Example 1) lead to significant improvement of pseudo-virus neutralization potencies. Thus, these improvements in IC50 range between 8 and 100-fold with Method 1, and between at least 8 and at least 300-fold with Method 2. A more detailed structure-activity relationship analysis of data in Table 6 also reveals interesting patterns, which are in complete agreement with the analysis of binding data (Table 5) as described in the previous section. Among these patterns, it is important to highlight the surprising and unexpected finding that the natural human variant Ile92 of ACE2 catalytic domain (R1 region) affords significant improvements in neutralization potency relative to the more common human variant having Thr residue at position 92 and consequently carbohydrate structure attached at Asn90. This is readily apparent from data in Table 6 and FIG. 8, if one compares the variant ACE2m1-SG4-Fc (SEQ ID NO:18) that contains Thr92 with variants ACE2m4-SG4-Fc (SEQ ID NO:21) and ACE2m4-hinge2CS-SG4-Fc (SEQ ID NO:23) that contain Ile92, while the rest of the ACE2 catalytic domain (R1 region) is identical between these variants. A surprising 4 to 5-fold (depending on the methods used) decrease in IC50 is thus directly attributable to the natural mutation T92I in human ACE2 catalytic domain.

TABLE 6
Neutralization of SARS-CoV-2 Wuhan
spike protein pseudo-typed virus.

| | SEQ | IC50 (ng/mL) | |
|---|---|---|---|
| Variant name | ID NO | Method 1 | Method 2 |
| ACE2-SG4-Fc | 15 | 2500 | >1000 |
| ACE2-SG4x3-Fc | 17 | 2500 | >1000 |
| ACE2m1-SG4-Fc | 18 | 95 | 17 |
| ACE2m2-SG4-Fc | 19 | 325 | 134 |
| ACE2m3-SG4-Fc | 20 | 191 | 58 |
| ACE2m4-SG4-Fc | 21 | 26 | 3 |
| ACE2m4-C261S-SG4-Fc | 22 | 73 | 12 |
| ACE2m4-hinge2CS-SG4-Fc | 23 | 25 | 3 |

Neutralization of VLPs Pseudo-Typed with Various SARS-CoV-2 Variants

The ability to neutralized VLPs pseudo-types with the S-protein from the original Wuhan SARS-CoV-2 prompted us to test select polypeptide of this invention for their ability to block cellular entry of VLPs pseudo-typed with S-proteins from other SARS-CoV-2 variants. The Method 1 co-expressing human ACE2 and TMPRSS2 on the HEK293T cells was employed for this assay. Pseudotyped SARS-CoV-2 spike lentiviral particles were produced using plasmids expressing various variants of the SARS-CoV-2 spike protein according to the protocols and reagents described by the Bloom lab [45], with the following modifications: (1) HEK293SF-3F6 cells [46]were used for large-scale production of lentiviral particles in 300 mL; (2) post-transfection HEK293SF-3F6 cells were incubated at 33° C. for improved yield; (3) 72 h post-infection lentiviral particles were harvested and subjected to concentration by sucrose cushion centrifugation. Briefly, the supernatant was placed on 20% sucrose cushion and spun for 3 h at 37,000×g at 4° C. The pellet containing the concentrated pseudo typed VLP was then resuspended in DMEM with 10% FBS and aliquoted. Titration was performed using HEK293T cells overexpressing human ACE2 and TMPRSS2, obtained from BEI Resources repository of ATCC and the NIH (NR-55293). The following plasmids were used. Wuhan: plasmid name=“pHDM SARS-Cov-2” (BEI Resources, NIAID, NIH: SARS-Related Coronavirus 2, Wuhan-Hu-1 Spike-Pseudotyped Lentiviral Kit, NR-52948). D614G: plasmid name=“pHDM SARS2-Spike-Del21-D614G”, HDM_SARSl_Spike_de121_D614G was a gift from Jesse Bloom (Addgene plasmid #: 158762; http://n2t.net/addgene:158762; RRID: Addgene_158762). B.1.1.7 (Alpha): plasmid name=“pPACK-SPIKE N501Y”, SARS-CoV-2 “S” Pseudo type—N501Y Mutant—Lenti vector Packaging Mix (SBI System Biosciences SBI, Cat #: CVD19-560A-1; Mutation: N501Y). B.1.351 (Beta): plasmid name=“pPACK-SPIKE B.1.351”, RBD Mutations Lentivector Packaging Mix (SBI System Biosciences, Cat #: CVD19-580A-1; Mutations: K417N, E484K and N501Y). B.1.617.2 (Delta): plasmid name=“pcDNA3.3-SARS2-B.1.617.2) pcDNA3.3-SARS2-B.1.617.2” was a gift from David Nemazee (Addgene plasmid #: 172320; http://n2t.net/addaene:172320; RRID: Addgene_172320).

Pseudovirus neutralization assay was performed according to the previously described protocol [45] and was adapted for 384-well plate. Briefly, 3-fold serial dilutions of the samples containing select polypeptides of this invention were incubated with diluted virus at a 1:1 ratio for 1 h at 37° C. before addition to HEK293-ACE2/TMPRSS2 cells. Infectivity was then measured by luminescence readout per well. Bright-Glo luciferase reagent (Promega, E2620) was added to wells for 2 min before reading with a PerkinElmer Envision instrument. 50% inhibitory concentration (IC50) were calculated with nonlinear regression (log[inhibitor] versus normalized response−variable slope) using GraphPad Prism 8 (GraphPad Software Inc.).

The tested polypeptides provided in the present invention, as shown in FIG. 8C, afford excellent blocking of cellular infection against the VLPs pseudo-typed with all the investigated viral S-protein variants. Associated IC50 values for various virus variants are listed in Table 6a (in nM and ng/mL). It is directly apparent that there is a little variation in the IC50 values across virus variants for each of the tested polypeptides of this invention. Noteworthy, there appears that both polypeptides of this invention tested neutralize the B.1.617.2 (Delta) variant, a major variant of concern, more potently (by 2-fold) than the original Wuhan virus. Taken together, this pseudo-virus neutralization data further support the pan-specificity of the class of polypeptides of the present invention that can afford a robust approach towards mitigating COVID-19 infections caused by current as well as future emerging variants of SARS-CoV-2.

TABLE 6a
Neutralization of VLP pseudo-typed with the spike
proteins from the SARS-CoV-2 variants of concern.

| | ACE2m4- | | ACE2l92- | |
|---|---|---|---|---|
| | hinge2CS-SG4-Fc | | hinge2CS-SG4-Fc | |
| | (SEQ ID NO: 23) | | (SEQ ID NO: 28) | |
| SARS-CoV-2 | IC50 | IC50 | IC50 | IC50 |
| variant | (ng/mL) | (nM) | (ng/mL) | (nM) |
| Wuhan | 24.19 | 0.127 | 1013 | 5.320 |
| | (±2.48) | (±0.013) | (±243) | (±1.277) |
| D614G | 62.48 | 0.328 | 1999 | 10.495 |
| | (±8.95) | (±0.047) | (±700) | (±3.677) |
| B.1.1.7 (Alpha) | 27.43 | 0.144 | na | na |
| | (±3.62) | (±0.019) | | |
| B.1.351 (Beta) | 39.81 | 0.209 | 1143 | 5.999 |
| | (±8.19) | (±0.043) | (±237) | (±1.242) |
| B.1.617.2 (Delta) | 10.10 | 0.053 | 581 | 3.051 |
| | (±1.33) | (±0.007) | (±268) | (±1.407) |

na: not assessed

### Example 8. SARS-CoV-2 Authentic Virus Neutralization by Polypeptide Constructs

The ability of polypeptides of this invention to neutralize infection of human VERO-E6 cells by the live replicating authentic virus was also assessed. This was done with a microneutralization assay. SARS-CoV-2 isolate Canada/ON/VIDO-01/2020 was obtained from the National Microbiology Laboratory (Winnipeg, MB, Canada) and propagated on Vero E6 cells and quantified on Vero cells. Whole viral genome sequencing was carried out to confirm exact genetic identity to original isolate. Passage 3 virus stocks were used. Neutralization activity was determined with the microneutralization assay. In brief, 1:5 serial dilutions of 15 μg of each polypeptide was carried out in DMEM, high glucose media supplemented with 1 mM sodium pyruvate, 1 mM non-essential amino acids, 100 U/mL penicillin-streptomycin, and 0.1% bovine serum albumin (BSA). SARS-CoV-2 was added at 125 plaque forming units (PFU) in 1:1 ratio to each antibody dilution and incubated at 37° C. for 1 h. After incubation, Vero E6 cells seeded in 96-well plates were infected with virus/polypeptide mix and incubated at 37° C. in humidified/5% CO₂ incubator for 72 h post-infection (hpi). Cells were then fixed in 10% formaldehyde overnight and virus infection was detected with mouse anti-SARS-CoV-2 nucleocapsid antibody (R&D Systems, clone #1035111) and counterstained with rabbit anti-mouse IgG-HRP (Rockland Inc.). Colorimetric development was obtained with o-phenylenediamine dihydrochloride peroxidate substrate (Sigma-Aldrich) and detected on Biotek Synergy H1 plate reader at 490 nm. IC50 was determined from non-linear regression using GraphPad Prism 9 software.

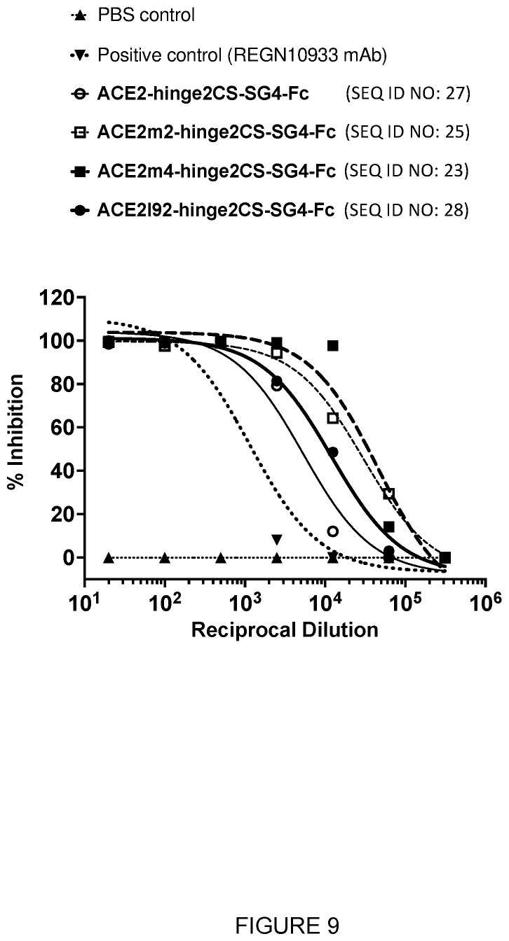

As shown in FIG. 9 and listed in Table 7, select polypeptides tested inhibited replication of live virus with IC50 values in the 1-4 ng/mL range, and afforded over 10-fold improved efficacies relative to the positive control anti-SARS-CoV-2 monoclonal antibody REGN10933 [47]. For example, the polypeptide ACE2m4-hinge2CS-SG4-Fc (SEQ ID NO: 23) inhibited infection of human VERO-E6 cells by authentic SARS-CoV-2 virus in cell culture in vitro, with an excellent IC50 around 1 ng/mL or 6 pM. Also, the polypeptide ACE2I92-hinge2CS-SG4-Fc (SEQ ID NO:28) inhibited infection of human VERO-E6 cells by authentic SARS-CoV-2 virus in cell culture in vitro, with a good IC50 around 4 ng/mL or 22 pM. By comparison, in this assay the control antibody REGN10933 displayed an IC50 of 43 ng/mL or 287 pM. Taken together, these data demonstrate that ADAPT-guided optimization of the ACE2 interface with SARS-CoV-2 spike RBD for improved binding affinity can translate into a marked functional improvements at cellular level.

TABLE 7
Neutralization of SARS-CoV-2 Wuhan authentic live virus.

| | |
|---|---|
| SEQ | IC50 |

| Variant name | ID NO | (ng/mL) | (nM) |
|---|---|---|---|
| ACE2-hinge2CS-SG4-Fc | 27 | 9.43 | 0.050 |
| ACE2l92-hinge2CS-SG4-Fc | 28 | 4.15 | 0.022 |
| ACE2m2-hinge2CS-SG4-Fc | 25 | 1.68 | 0.009 |
| ACE2m4-hinge2CS-SG4-Fc | 23 | 1.08 | 0.006 |
| REGN10933 mAb | | 43.33 | 0.289 |

### Example 9. In Vivo Evaluation in Hypertensive Mice

[We gratefully acknowledge that some of the technical data relating to in vivo evaluation in hypertensive mice were kindly provided by the lab of Dr. Kevin Burns (OHRI, University of Ottawa, Ottawa, Canada)].

### Ethics Statement

All experiments conducted on animals were approved by the University of Ottawa Animal Care Committee (protocol 3514), following regulations of the Canadian Council on Animal Care (CCAC).

### Allocation of Interventions

Mice were housed at the University of Ottawa Animal Care facility with humidity and temperature constantly monitored, and with free access to water and chow. The allocation of cages was conducted at random within the shelves. Allocation to experimental groups was performed using an online randomization tool (randomizer.org).

### Angiotensin II Infusion

12-14 week old C57BL/6 male and female C57BL/6 mice were subjected to subcutaneous implantation of osmotic minipumps (Alzet®, model 1004). Mice were injected with 0.01 mg/kg buprenorphine 1 h prior to the minipump implantation. Mice were initially anesthetized with 5% isoflurane, and kept at 2.5% isofluorane throughout surgery. Animals were shaved and skin was sterilized with solution containing chlorhexidine gluconate 4%. A 0.5 cm wide incision was made perpendicular to the spine, in the subcapsular area, and a subcutaneous pocket was made using a haemostat. Minipumps were inserted in the pocket and the incision was closed using 2 suture wound clips. Topical bupivacaine was applied to the incision and animals were transferred to a heated recovery area, until they recovered from anesthesia. Mice received another injection of buprenorphine 4 h after surgery and were monitored for 72 h, twice daily. Angiotensin II was infused for 3 weeks at 1000 ng/kg/min, and control mice were infused with saline solution via minipump.

### Intravenous Administration of Polypeptides of the Present Invention

Two polypeptides of the present invention were administered to mice intravenously: ACE2m4-hinge2CS-SG4-Fc (SEQ ID NO: 23) and ACE2I92-hinge2CS-SG4-Fc (SEQ ID NO: 28). Two categories of mice were studied: normotensive and hypertensive. Mice from were allocated into groups: i) Saline, ii) Ang II, iii) Ang II+rhACE2, iv) Ang II+ACE2m4-hinge2CS-SG4-Fc, v) Ang II+ACE2I92-hinge2CS-SG4-Fc. Mice received a single intravenous injection (via tail vein) of variants ACE2m4-hinge2CS-SG4-Fc (10 mg/kg), ACE2I92-hinge2CS-SG4-Fc (10 mg/kg), or rhACE2 (2.5 mg/kg—BioLegend, Cat #: 792008) at 2.5 weeks after osmotic minipump insertion. Control mice received PBS. Euthanasia occurred 72 h after injection of polypeptides of this invention or rhACE2, and plasma, kidneys, heart, lungs, liver, and spleen were collected for analyses.

### Blood Pressure Measurement

Systolic blood pressure (SBP) was measured by tail-cuff plethysmography (BP-2000; Visitech Systems, Apex, NC), as previously described [48]. Mice underwent training for 5 consecutive days and mock measurements were taken. SBP was assessed at baseline (3 measurements), 2.5 weeks after osmotic minipump implantation (2-3 measurements), and 3, 6, 24, 48, and 72 h after injection of polypeptides of this invention. Each mouse was subjected to 10 SBP readings per timepoint, and the mean SBP was calculated from these readings.

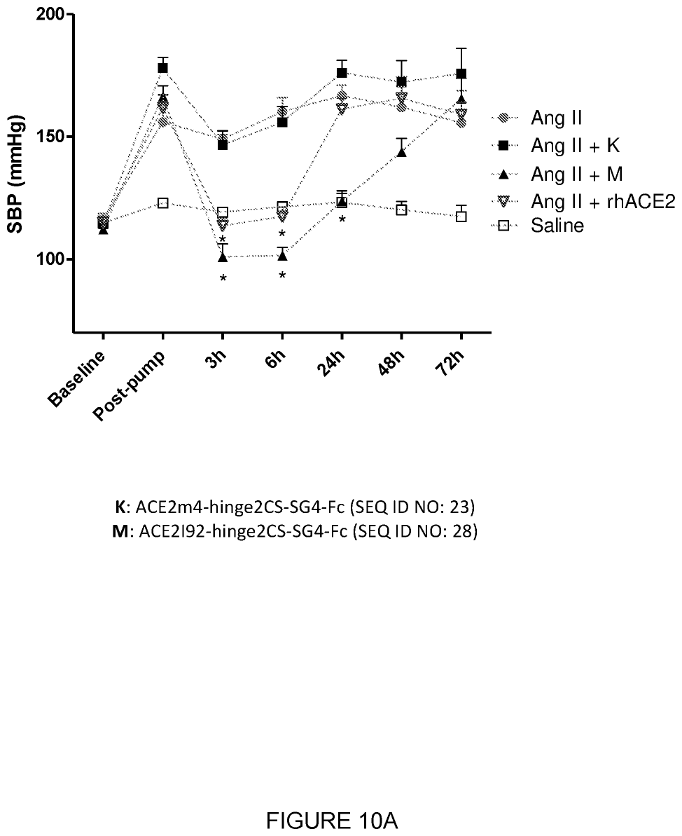

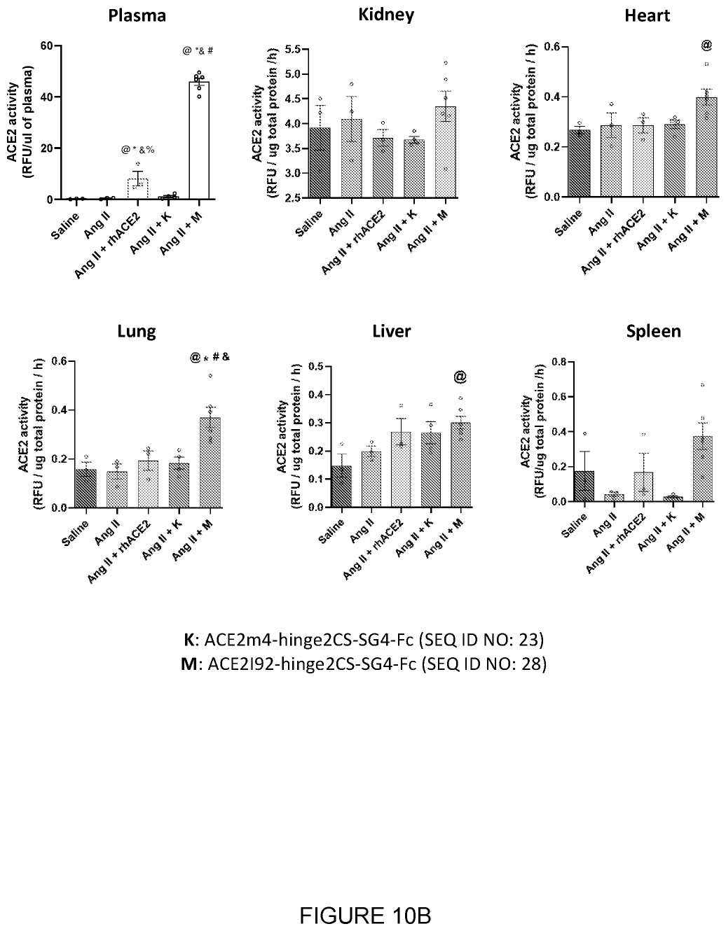

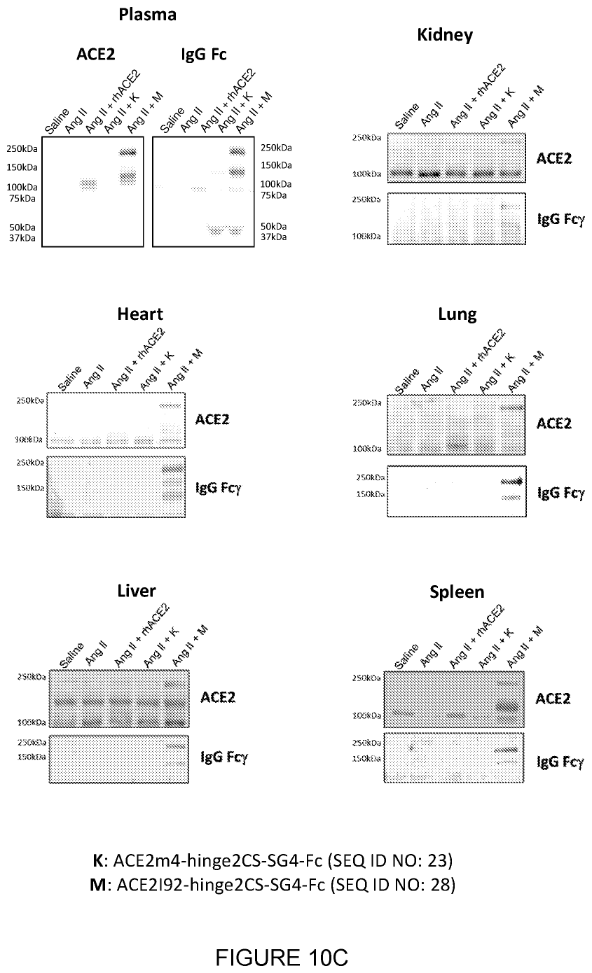

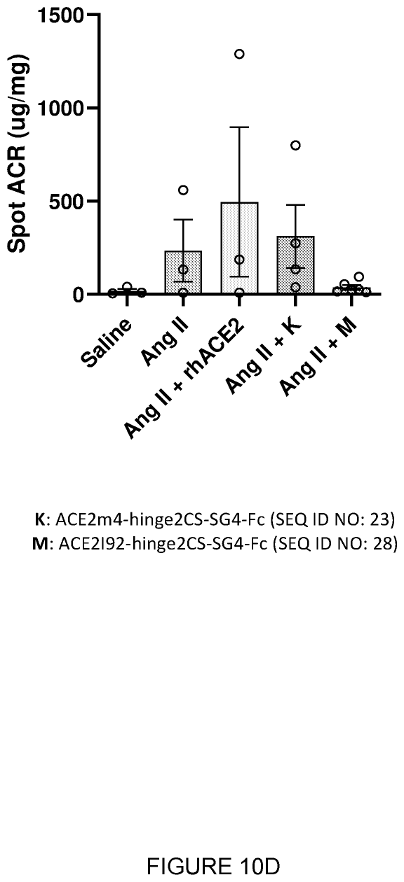

Hypertension induced by angiotensin II infusion was confirmed by a significant elevation in SBP 2.5 weeks after osmotic minipump implantation. rhACE2 transiently reduced SBP up to 6 h after injection, whereas ACE2m4-hinge2CS-SG4-Fc (SEQ ID NO: 23) had no significant effect on SBP (FIG. 10A). Noteworthy, ACE2I92-hinge2CS-SG4-Fc (SEQ ID NO: 28) significantly reduced SBP for up to 24 h, and this lowering effect was partially sustained for 48 h (FIG. 10A).

### Plasma and Tissue ACE2 Activity

ACE2 enzymatic activity was assessed in plasma and tissues (kidney, heart, lung, liver, and spleen) collected at the endpoint (72 h). Activity was determined with the fluorogenic substrate Mca-APK(Dnp) from Anaspec, with or without the ACE2 specific inhibitor MLN-4760, as described [40]. Plasma ACE2 activity was determined in 5 μL of plasma. For tissue ACE2 activity, the samples were homogenized in 500 μL of lysis buffer containing 50 mM HEPES, pH 7.4, 150 mM NaCl, 0.5% Triton X-100, 0.025 mM ZnCl₂, and protease inhibitor cocktail (Sigma-Aldrich, Cat #: P8340). Lysates were centrifugated at 12,000×g for 10 min at 4° C. to remove debris. The supernatants were stored at −80° C. Protein concentration was determined using DC protein assay kit (Bio-Rad Laboratories, Cat #: 5000112). Due to differences in ACE2 abundance, kidney tissue was assayed at 1 μg total protein and incubated for 1 h with the fluorogenic substrate. Heart, lung, liver, and spleen ACE2 activities were assayed at 10 μg, incubated with the fluorogenic substrate (11.25 μM) for 16 h. Data are presented as Mean±SEM. Statistical analyses were performed using Prism 9.3.0 for Windows, GraphPad Software, San Diego, California USA, www.graphpad.com. Comparison amongst groups was performed by One-way ANOVA, followed by either Tukey's or Dunnett's post-test. P<0.05 was considered statistically significant.

ACE2 activity was assessed in plasma collected from mice at endpoint (72 h after administration of polypeptides of this invention). In this assay, ACE2m4-hinge2CS-SG4-Fc (SEQ ID NO; 23) was virtually inactive in plasma at endpoint, whereas activity of ACE2I92-hinge2CS-SG4-Fc (SEQ ID NO: 28) was significantly increased compared to all groups (FIG. 10B).

Tissue ACE2 activity was analyzed in lysates of kidney, heart, lung, liver, and spleen (FIG. 10B). There was no significant difference in activity in kidney lysates amongst all samples. ACE2 activity was significantly increased in the heart lysates of Ang II+ACE2m4-hinge2CS-SG4-Fc group. ACE2 activity was significantly higher in the liver lysate of group Ang II+ACE2m4-hinge2CS-SG4-Fc. Lastly, when samples from spleen were analyzed, there was no difference in ACE2 activity amongst the groups (FIG. 8A).

### Immunoblotting

Tissue lysate was obtained as described above. The amount of lysate varied depending on the tissue/sample type, as follows: plasma 1 μl, kidney 20 μg, heart and lung 30 μg, liver and spleen 40 μg. The lysates were added to 4× Laemmli sample buffer, loaded into a gradient SDS-PAGE gel (5-15%), and submitted to electrophoresis. Protein was transferred from the gel to a nitrocellulose membrane (Bio-Rad Laboratories, Cat #: 1620112), and blocked for 1 h in Tris-buffered saline (pH 7.6) solution containing 0.1% Tween 20 (TBS-T) and 3% bovine serum albumin (BSA), for 1 h at room temperature. Membranes were probed with goat anti-ACE2 (1:1000 dilution, R&D systems, Cat #: AF933) overnight at 4° C., followed by incubation with 1:5,000 HRP-donkey anti-goat IgG (Jackson ImmunoResearch, Cat #: 705-035-147). Probing for IgG Fc was done using HRP-donkey anti-human IgG Fcγ 1:10,000 (Jackson ImmunoResearch, Cat #: 709-035-098), overnight at 4° C. Chemiluminescence was induced by adding Amersham ECL Western Blotting Detection Reagents to the membranes (GE Healthcare, Cat #: CA95038-564L), and detected on Alpha Innotech FluorChem Q Quantitative Western Blot Imaging System.

ACE2 was detected by immunoblotting in the plasma of rhACE2 and the Ang II+ACE2I92-hinge2CS-SG4-Fc (SEQ ID NO: 28) groups, and to a very low level in the plasma of the Ang II+ACE2m4-hinge2CS-SG4-Fc (SEQ ID NO: 23) group (FIG. 10C). In addition, ACE2 was detected in lysates from many organ tissues (FIG. 10C) from several groups. In most samples, immunoblotting detection (FIG. 10C) correlated with the measured ACE2 activity (FIG. 10B). Notably, immunoblots for ACE2 in kidney showed substantial levels of endogenous ACE2 (˜100 kDa), and a less pronounced high molecular weight band (immediately below 250 kDa; which may correspond to an ACE2-Fc homodimer of the present invention) was detected only in the lysates from the Ang II+ACE2I92-hinge2CS-SG4-Fc group. When probed with anti-human IgG Fcγ, the same band was detected, indicating the presence of the ACE2I92-hinge2CS-SG4-Fc in kidney tissue lysates. The immunoblots for ACE2 and IgG Fcγ were consistent with ACE2 activity data in heart lysates, showing considerable retention of ACE2I92-hinge2CS-SG4-Fc in heart tissue. Lung lysates also demonstrated persistent presence of the ACE2I92-hinge2CS-SG4-Fc. Furthermore, the ratio ACE2I92-hinge2CS-SG4-Fc to endogenous ACE2 was elevated. In liver lysates, the high MW band for ACE2I92-hinge2CS-SG4-Fc was again detected on immunoblot. There was a strong signal for the high MW band corresponding to ACE2I92-hinge2CS-SG4-Fc on immunoblots from spleen.

### Albumin to Creatinine Ratio

Spot urine was collected at the endpoint (72 h). Urine samples were diluted 1:200 to 1:500, following manufacturer instructions. Albumin was measured by ELISA (Bethyl Laboratories Inc., Cat #: E99-134). Creatinine was quantified in the urine using a colorimetric based kit by Ethos Bioscience (Cat #: 1012). Values of albumin were converted to g/dL and ratio albumin to creatinine was calculated by dividing albumin levels by creatinine levels (mg/dL).

The increase in urinary excretion of albumin can indicate kidney damage. Analysis using urine collected at the endpoint (72 h) showed that there were no significant changes in albumin to creatinine ratio (ACR). However, there was a clear trend to increase in the hypertensive groups, compare to Saline group. Hypertensive mice treated with ACE2I92-hinge2CS-SG4-Fc (SEQ ID NO: 28) had the lowest values for albumin excretion, comparable to Saline group (FIG. 10D).

In conclusion, although many novel polypeptides of the present invention showed sustained in vitro ACE2 activity, with performance comparable to rhACE2, polypeptide ACE2m4-hinge2CS-SG4-Fc (SEQ ID NO: 23) rapidly lost activity in vivo. The candidate polypeptide ACE2I92-hinge2CS-SG4-Fc (SEQ ID NO: 28) had enhanced performance in vivo in this model, transiently reducing SBP. Persistent detection of ACE2I92-hinge2CS-SG4-Fc (SEQ ID NO: 28) in various tissue lysates 72 h after i.v. injection, along with increased ACE2 activity and prolonged blood pressure lowering-effect, suggest that ACE2I92-hinge2CS-SG4-Fc (SEQ ID NO: 28) may have significant therapeutic potential. Taken together, the ACE2 enzyme activity and immunoblotting in plasma and various tissues are congruent and support SBP lowering effects as well as reduced albumin urea observed with ACE2I92-hinge2CS-SG4-Fc (SEQ ID NO: 28) and strongly suggest therapeutic effects of select polypeptides of the present invention as protective agents against major organ injuries caused by COVID-19.

### Example 10. In Vivo Evaluation in SARS-CoV-2 Infected Hamsters

### Intranasal Administration

Given instability of some polypeptides of the present invention in plasma observed in the in vivo model of hypertensive mice, an intranasal route of administration was adopted first for in vivo testing the recovery from COVID-19 using a hamster model [49]. Male Syrian golden hamsters (81-90 g) were obtained from Charles River Laboratories (Saint-Constant, QC, Canada). Animals were maintained at the animal facility of the National Research Council Canada (NRC) in accordance with the guidelines of the Canadian Council on Animal Care. All procedures performed on animals in this study were in accordance with regulations and guidelines reviewed and approved in animal use protocol 2020.06 by the NRC Human Health Therapeutics Animal Care Committee. Male hamsters were challenged intranasally with 8×10³ PFU of SARS-CoV-2 isolate Canada/ON/VIDO-01/2020. Two dosing regimens were tested. In a combined prophylactic and therapeutic dosing, animals were intranasally administered polypeptides of the present invention 4 hours before viral challenge and also a repeat dose 24 after viral challenge. In a therapeutic only dosing, animals were intranasally administered polypeptides of the present invention 6 hours after viral challenge and repeat doses were administered at 24 h intervals. All intranasal administrations were performed with a fixed total volume of 150 μL, alternating between the left and right nostrils until each nostril received a total of 75 μL of test article solution. Control group of animals received PBS vehicle only. Daily body weights were determined. Data were presented as Mean±SEM. n=6 to 7 animals per group.

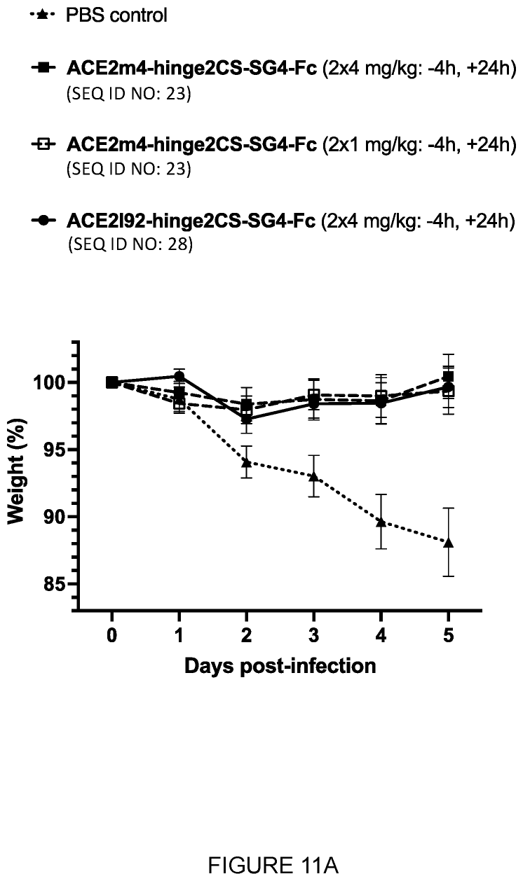

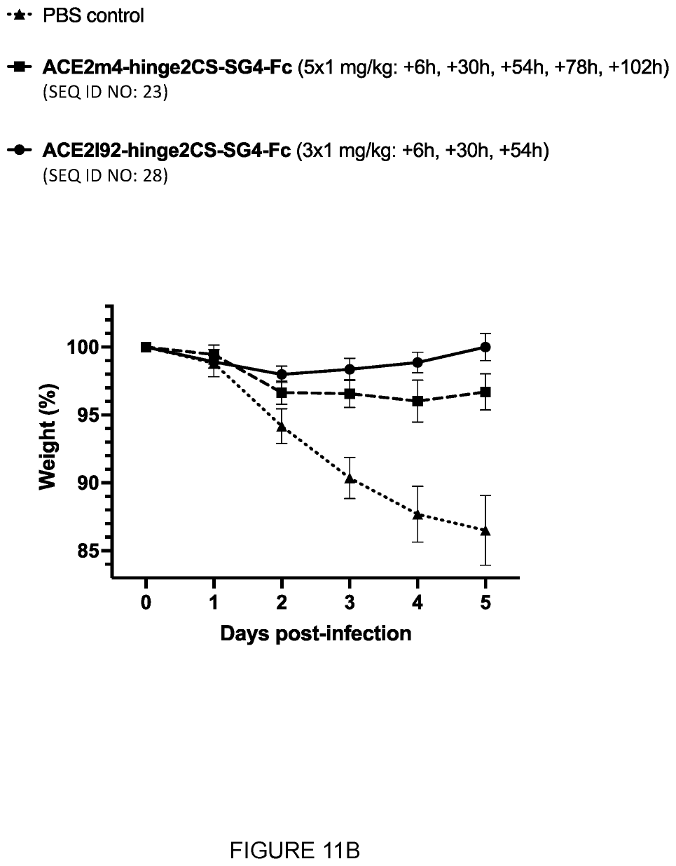

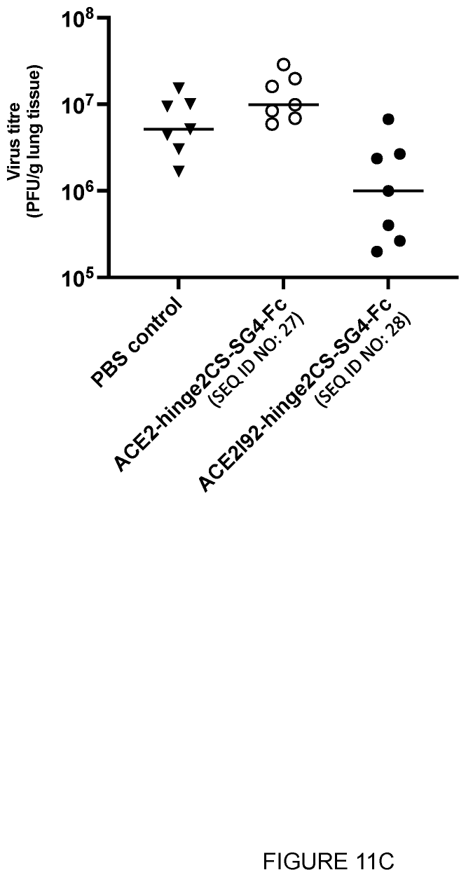

Body weight changes for the various groups following combined prophylactic and therapeutic administration are shown in FIG. 11A. Both polypeptides of this invention tested ACE2m4-hinge2CS-SG4-Fc (SEQ ID NO: 23) and ACE2I92-hinge2CS-SG4-Fc (SEQ ID NO: 28), had a marked impact on the treated groups relative to the control group (vehicle alone), as indicated by reduced body weight losses. For both tested articles, the recovery actually led to body weight gains at day 3 post-challenge. Moreover, similar recovery with the ACE2m4-hinge2CS-SG4-Fc polypeptide construct were attained at the two doses tested, a high dose of 4 mg/kg (administered twice) as well as a low dose of 1 mg/kg (administered twice). These results demonstrate the viability of treating COVID-19 with polypeptides of these invention, including but not limited to ACE2m4-hinge2CS-SG4-Fc (SEQ ID NO: 23), via a non-systemic administration route.

Body weight changes for the various groups following therapeutic only administration are shown in FIG. 11B. Both polypeptides of this invention tested ACE2m4-hinge2CS-SG4-Fc (SEQ ID NO: 23) and ACE2I92-hinge2CS-SG4-Fc (SEQ ID NO: 28), had a marked impact on the treated groups relative to the control group (vehicle alone), as indicated by reduced body weight losses. The ACE2I92-hinge2CS-SG4-Fc polypeptide construct led to a more pronounced recovery even at the lower number of administrations than the ACE2m4-hinge2CS-SG4-Fc polypeptide construct (3 versus 5 therapeutic administrations of 1 mg/kg, respectively). Moreover, the recovery actually led to body weight gains after day 2 post-challenge with the ACE2I92-hinge2CS-SG4-Fc treatment, and day 4 post-challenge with the ACE2m4-hinge2CS-SG4 treatment. These results demonstrate further support the intended use polypeptides of these invention as therapeutic agents for recovery from SARS-CoV-2 viral infection and COVID-19.

### Intravenous Administration

Since ACE2I92-hinge2CS-SG4-Fc (SEQ ID NO: 28) exhibited excellent stability in plasma nd organ tissues including lung and other organs (FIG. 10), it was also evaluated with a systemic route of administration for in vivo in the COVID-19 hamster model [49]. Female Golden Syrian hamsters 81-90 g (Charles River Labs) were infected intranasally with 10⁴ PFU of SARS-CoV-2 live virus. Select polypeptides of the present invention in PBS solution were administered in the retro-orbital vein at a dose of 10 mg/Kg at the time of viral challenge. A repeat administration with the same dose was done 24 h post viral challenge. A control group of animals received PBS only. Each group consisted of 7 animals. Animals were euthanized at 3 days post-challenge and viral load was determined for lung tissues by plaque assay. Animals were euthanized at 3 days post-challenge and viral load in lung tissues was determined with the plaque assay by infection of cultured VERO-E6 cells as previously described [50].

As shown in FIG. 11C, i.v. administration of ACE2I92-hinge2CS-SG4-Fc (SEQ ID NO: 28) reduced SARS-CoV-2 infection in female hamsters, as evidenced by median levels of live virus determined by plaque assay being reduced by almost one order of magnitude, relative to the median level in the PBS control group. Importantly, it is apparent from the data shown in FIG. 11C that the T92I mutation in the ACE2 ectodomain region of the polypeptides of this invention was key to achieving reduction in viral titre. This is evidenced by a comparison with the ACE2-hinge2CS-SG4-Fc (SEQ ID NO: 27) polypeptide incorporating the parental unmutated human ACE2 ectodomain, which does not lead to a median reduction of viral titre relative to the median viral titre in PBS control group, and on the contrary seems to have quite an the opposite effect. This data underscores the unique structure of the polypeptide fusions of this invention based on the T92I naturally occurring mutation of the human ACE2 as a necessary key element with surprising and unexpected efficacy profile against COVID-19 infection and the associated major organ injuries.

Sequence listing

| SEQ | | |
|---|---|---|
| ID | | |
| NO: | Name/Description | Sequence |
| 1 | R1-wt | QSTIEEQAKTFLDKFNHEAEDLFYQSSLASWNYNTNITEENVQNMN |
| | (hACE2) | NAGDKWSAFLKEQSTLAQMYPLQEIQNLTVKLQLQALQQNGSSVLS |
| | | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGREWTNLYSLTVPFGQKPNIDV |
| | | TDAMVDQAWDAQRIFKEAEKFFVSVGLPNMTQGFWENSMLTDPGNV |
| | | QKAVCHPTAWDLGKGDFRILMCTKVTMDDELTAHHEMGHIQYDMAY |
| | | AAQPELLRNGANEGFHEAVGEIMSLSAATPKHLKSIGLLSPDFQED |
| | | NETEINFLLKQALTIVGTLPFTYMLEKWRWMVFKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 2 | R1-wt-192 | QSTIEEQAKTFLDKFNHEAEDLFYQSSLASWNYNTNITEENVQNMN |
| | (hACE2192) | NAGDKWSAFLKEQSTLAQMYPLQEIQNLIVKLQLQALQQNGSSVLS |
| | | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGREWTNLYSLTVPFGQKPNIDV |
| | | TDAMVDQAWDAQRIFKEAEKFFVSVGLPNMTQGFWENSMLTDPGNV |
| | | QKAVCHPTAWDLGKGDFRILMCTKVTMDDFLTAHHEMGHIQYDMAY |
| | | AAQPFLLRNGANEGFHEAVGEIMSLSAATPKHLKSIGLLSPDFQED |
| | | NETEINFLLKQALTIVGTLPFTYMLEKWRWMVEKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 3 | R1-m1 | QSTIEEQAKYFLDKFNHEAEDLFYQSSLASWNYNTNITEENVQNMN |
| | (m1: hACE2- | NAGDKWSAFLKEQSTLAQMYPLQEIQNLTVKLQLQALQQNGSSVLS |
| | T27Y, N330Y) | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGREWTNLYSLTVPFGQKPNIDV |
| | | TDAMVDQAWDAQRIFKEAEKFFVSVGLPNMTQGFWEYSMLTDPGNV |
| | | QKAVCHPTAWDLGKGDERILMCTKVTMDDELTAHHEMGHIQYDMAY |
| | | AAQPFLLRNGANEGFHEAVGEIMSLSAATPKHLKSIGLLSPDFQED |
| | | NETEINFLLKQALTIVGTLPFTYMLEKWRWMVEKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 4 | R1-m2 | QSTIEEQAKYFLDKFNHEAEDLFYQSSLASWNYNTNITEENVQNMN |
| | (m2: hACE2192-T27Y) | NAGDKWSAFLKEQSTLAQMYPLQEIQNLIVKLQLQALQQNGSSVLS |
| | | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGRFWTNLYSLTVPFGQKPNIDV |
| | | TDAMVDQAWDAQRIFKEAEKFFVSVGLPNMTQGFWENSMLTDPGNV |
| | | QKAVCHPTAWDLGKGDFRILMCTKVTMDDELTAHHEMGHIQYDMAY |
| | | AAQPFLLRNGANEGFHEAVGEIMSLSAATPKHLKSIGLLSPDFQED |
| | | NETEINFLLKQALTIVGTLPFTYMLEKWRWMVFKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 5 | R1-m3 | QSTIEEQAKTFLDKFNHEAEDLFYQSSLASWNYNTNITEENVQNMN |
| | (m3: hACE2192- | NAGDKWSAFLKEQSTLAQMYPLQEIQNLIVKLQLQALQQNGSSVLS |
| | N330Y) | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGREWTNLYSLTVPFGQKPNIDV |
| | | TDAMVDQAWDAQRIFKEAEKFFVSVGLPNMTQGFWEYSMLTDPGNV |
| | | QKAVCHPTAWDLGKGDFRILMCTKVTMDDELTAHHEMGHIQYDMAY |
| | | AAQPELLRNGANEGFHEAVGEIMSLSAATPKHLKSIGLLSPDFQED |
| | | NETEINFLLKQALTIVGTLPFTYMLEKWRWMVFKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 6 | R1-m4 | QSTIEEQAKYFLDKFNHEAEDLFYQSSLASWNYNTNITEENVQNMN |
| | (m4: hACE2192- | NAGDKWSAFLKEQSTLAQMYPLQEIQNLIVKLQLQALQQNGSSVLS |
| | T27Y,N330Y) | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGCLPAHLLGDMWGREWTNLYSLTVPFGQKPNIDV |
| | | TDAMVDQAWDAQRIFKEAEKFFVSVGLPNMTQGFWEYSMLTDPGNV |
| | | QKAVCHPTAWDLGKGDERILMCTKVTMDDELTAHHEMGHIQYDMAY |
| | | AAQPELLRNGANEGFHEAVGEIMSLSAATPKHLKSIGLLSPDFQED |
| | | NETEINFLLKQALTIVGTLPFTYMLEKWRWMVEKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 7 | R1-m4-C261S | QSTIEEQAKYFLDKFNHEAEDLFYQSSLASWNYNTNITEENVQNMN |
| | (m4: hACE2192- | NAGDKWSAFLKEQSTLAQMYPLQEIQNLIVKLQLQALQQNGSSVLS |
| | T27Y,C261S,N330Y) | EDKSKRLNTILNTMSTIYSTGKVCNPDNPQECLLLEPGLNEIMANS |
| | | LDYNERLWAWESWRSEVGKQLRPLYEEYVVLKNEMARANHYEDYGD |
| | | YWRGDYEVNGVDGYDYSRGQLIEDVEHTFEEIKPLYEHLHAYVRAK |
| | | LMNAYPSYISPIGSLPAHLLGDMWGREWTNLYSLTVPFGQKPNIDV |
| | | TDAMVDQAWDAQRIFKEAEKFFVSVGLPNMTQGFWEYSMLTDPGNV |
| | | QKAVCHPTAWDLGKGDFRILMCTKVTMDDELTAHHEMGHIQYDMAY |
| | | AAQPELLRNGANEGFHEAVGEIMSLSAATPKHLKSIGLLSPDFQED |
| | | NETEINFLLKQALTIVGTLPFTYMLEKWRWMVFKGEIPKDQWMKKW |
| | | WEMKREIVGVVEPVPHDETYCDPASLFHVSNDYSFIRYYTRTLYQF |
| | | QFQEALCQAAKHEGPLHKCDISNSTEAGQKLENMLRLGKSEPWTLA |
| | | LENVVGAKNMNVRPLLNYFEPLFTWLKDQNKNSFVGWSTDWSPYA |
| 8 | R2-SG4 | SGGGG |
| 9 | R2-SG4x3 | SGGGGSGGGGSGGGG |
| 10 | R3-wt | EPKSCDKTHTCPPCP |
| 11 | R3-S220 | EPKSSDKTHTCPPCP |
| 12 | R3-S220hinge2CS | EPKSSDKTHTSPPSP |
| 13 | R4-wt | APELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPG |
| 14 | R4-G270 | APELLGGPSVFLFPPKPKDTLMISRTPEVTCVVVDVSHEGPEVKFN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPG |
| 15 | ACE2-SG4-Fc | MSSSSWLLLSLVAVTAAQSTIEEQAKTFLDKFNHEAEDLFYQSSLA |
| | | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTF |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 16 | ACE2192-SG4-Fc | MSSSSWLLLSLVAVTAAQSTIEEQAKTELDKFNHEAEDLFYQSSLA |
| | | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 17 | ACE2-SG4x3-Fc | MSSSSWLLLSLVAVTAAQSTIEEQAKTELDKFNHEAEDLFYQSSLA |
| | | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGRFWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDERILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGSGGGGSGGGGEPKSSDKTHTCPPCP |
| | | APELLGGPSVFLFPPKPKDTLMISRTPEVTCVVVDVSHEGPEVKFN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPG |
| 18 | ACE2m1-SG4-Fc | MSSSSWLLLSLVAVTAAQSTIEEQAKYFLDKFNHEAEDLFYQSSLA |
| | | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGRFWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 19 | ACE2m2-SG4-Fc | MSSSSWLLLSLVAVTAAQSTIEEQAKYELDKFNHEAEDLFYQSSLA |
| | | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFS |
| | | CSVMHEALHNHYTQKSLSLSPG |
| 20 | ACE2m3-SG4-Fc | MSSSSWLLLSLVAVTAAQSTIEEQAKTELDKFNHEAEDLFYQSSLA |
| | | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDFRILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 21 | ACE2m4-SG4-Fc | MSSSSWLLLSLVAVTAAQSTIEEQAKYELDKFNHEAEDLFYQSSLA |
| | | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 22 | ACE2m4-C261S- | MSSSSWLLLSLVAVTAAQSTIEEQAKYELDKFNHEAEDLFYQSSLA |
| | SG4-Fc | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTF |
| | | EEIKPLYEHLHAYVRAKLMNAYPSYISPIGSLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDFRILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTCPPCPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 23 | ACE2m4-hinge2CS- | MSSSSWLLLSLVAVTAAQSTIEEQAKYELDKFNHEAEDLFYQSSLA |
| | SG4-Fc | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDERILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLENMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 24 | ACE2m3-hinge2CS- | MSSSSWLLLSLVAVTAAQSTIEEQAKTFLDKFNHEAEDLFYQSSLA |
| | SG4-Fc | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDERILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLENMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 25 | ACE2m2-hinge2CS- | MSSSSWLLLSLVAVTAAQSTIEEQAKYFLDKFNHEAEDLFYQSSLA |
| | SG4-Fc | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 26 | ACE2m1-hinge2CS- | MSSSSWLLLSLVAVTAAQSTIEEQAKYFLDKFNHEAEDLFYQSSLA |
| | SG4-Fc | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWEYSMLTDPGNVQKAVCHPTAWDLGKGDERILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 27 | ACE2-hinge2CS- | MSSSSWLLLSLVAVTAAQSTIEEQAKTELDKFNHEAEDLFYQSSLA |
| | SG4-Fc | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTF |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDERILMCTKVTMDD |
| | | FLTAHHEMGHIQYDMAYAAQPFLLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLENMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 28 | ACE2192-hinge2CS- | MSSSSWLLLSLVAVTAAQSTIEEQAKTELDKFNHEAEDLFYQSSLA |
| | SG4-Fc | SWNYNTNITEENVQNMNNAGDKWSAFLKEQSTLAQMYPLQEIQNLI |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTF |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLFNMLRLGKSEPWTLALENVVGAKNMNVRPLLNYFEPLFTWLKDQ |
| | | NKNSFVGWSTDWSPYASGGGGEPKSSDKTHTSPPSPAPELLGGPSV |
| | | FLFPPKPKDTLMISRTPEVTCVVVDVSHEGPEVKFNWYVDGVEVHN |
| | | AKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIA |
| | | VEWESNGQPENNYKTTPPVLDSDGSFFLYSKLTVDKSRWQQGNVES |
| | | CSVMHEALHNHYTQKSLSLSPG |
| 29 | ACE2-hinge2CS- | MSSSSWLLLSLVAVTAAQSTIEEQAKTELDKFNHEAEDLFYQSSLA |
| | SG4x3-Fc | SWNYNTNITEENVQNMNNAGDKWSAFLKEQSTLAQMYPLQEIQNLT |
| | | VKLQLQALQQNGSSVLSEDKSKRLNTILNTMSTIYSTGKVCNPDNP |
| | | QECLLLEPGLNEIMANSLDYNERLWAWESWRSEVGKQLRPLYEEYV |
| | | VLKNEMARANHYEDYGDYWRGDYEVNGVDGYDYSRGQLIEDVEHTE |
| | | EEIKPLYEHLHAYVRAKLMNAYPSYISPIGCLPAHLLGDMWGREWT |
| | | NLYSLTVPFGQKPNIDVTDAMVDQAWDAQRIFKEAEKFFVSVGLPN |
| | | MTQGFWENSMLTDPGNVQKAVCHPTAWDLGKGDFRILMCTKVTMDD |
| | | FLTAHHEMGHIQYDMAYAAQPELLRNGANEGFHEAVGEIMSLSAAT |
| | | PKHLKSIGLLSPDFQEDNETEINFLLKQALTIVGTLPFTYMLEKWR |
| | | WMVFKGEIPKDQWMKKWWEMKREIVGVVEPVPHDETYCDPASLFHV |
| | | SNDYSFIRYYTRTLYQFQFQEALCQAAKHEGPLHKCDISNSTEAGQ |
| | | KLENMLRLGKSEPWTLALENVVGAKNMNVRPLLNYFEPLFTWLKDQ |
| | | NKNSFVGWSTDWSPYASGGGGSGGGGSGGGGEPKSSDKTHTSPPSP |
| | | APELLGGPSVFLFPPKPKDTLMISRTPEVTCVVVDVSHEGPEVKEN |
| | | WYVDGVEVHNAKTKPREEQYNSTYRVVSVLTVLHQDWLNGKEYKCK |
| | | VSNKALPAPIEKTISKAKGQPREPQVYTLPPSRDELTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPG |
| 30 | ACE2m4-SG4-Fc | ATGTCCTCCTCAAGCTGGCTGCTGCTGTCTCTGGTGGCCGTGACCG |
| | (exemplary DNA | CCGCTCAGTCAACAATCGAAGAACAGGCAAAATACTTCCTGGACAA |
| | sequence) | GTTCAACCACGAGGCCGAGGATCTGTTTTATCAGTCCTCCCTGGCC |
| | | TCCTGGAACTACAATACCAACATCACAGAGGAGAATGTGCAGAACA |
| | | TGAACAATGCCGGCGACAAGTGGTCCGCCTTCCTGAAGGAGCAGTC |
| | | TACACTGGCCCAGATGTACCCCCTGCAGGAGATCCAGAATCTGATC |
| | | GTGAAGCTGCAGCTGCAGGCTCTGCAGCAGAACGGCTCTTCCGTGC |
| | | TGTCCGAGGATAAGAGCAAGCGCCTGAATACAATCCTGAACACCAT |
| | | GTCTACAATCTATTCCACCGGCAAGGTGTGCAATCCAGACAACCCC |
| | | CAGGAGTGTCTGCTGCTGGAGCCAGGCCTGAATGAGATCATGGCCA |
| | | ACTCTCTGGATTACAATGAGAGACTGTGGGCTTGGGAGTCCTGGCG |
| | | CAGCGAAGTGGGCAAGCAGCTGAGGCCCCTGTACGAGGAGTATGTG |
| | | GTGCTGAAGAACGAGATGGCCCGGGCTAATCACTACGAGGACTATG |
| | | GCGATTACTGGAGAGGCGACTACGAAGTGAATGGCGTGGACGGCTA |
| | | CGATTATTCTCGCGGCCAGCTGATCGAGGATGTGGAGCATACCTTT |
| | | GAGGAGATCAAGCCTCTGTATGAGCACCTGCATGCCTACGTGAGGG |
| | | CTAAGCTGATGAACGCCTATCCCTCTTACATCTCCCCTATCGGATG |
| | | CCTGCCAGCTCACCTGCTGGGCGACATGTGGGGCCGGTTCTGGACA |
| | | AATCTGTACTCCCTGACCGTGCCCTTTGGCCAGAAGCCTAACATCG |
| | | ACGTGACCGATGCCATGGTGGACCAGGCCTGGGATGCTCAGAGAAT |
| | | CTTCAAGGAGGCTGAGAAGTTCTTTGTGAGCGTGGGCCTGCCTAAC |
| | | ATGACACAGGGCTTTTGGGAGTACTCCATGCTGACCGACCCAGGCA |
| | | ACGTGCAGAAGGCCGTGTGCCACCCCACAGCTTGGGACCTGGGCAA |
| | | GGGCGATTTCCGCATCCTGATGTGCACCAAGGTGACAATGGACGAT |
| | | TTTCTGACCGCCCACCATGAGATGGGCCATATCCAGTATGATATGG |
| | | CTTACGCCGCTCAGCCATTCCTGCTGAGGAATGGCGCCAACGAGGG |
| | | CTTTCACGAGGCTGTGGGCGAGATCATGTCCCTGAGCGCCGCTACA |
| | | CCTAAGCATCTGAAGTCCATCGGCCTGCTGAGCCCAGACTTCCAGG |
| | | AGGATAACGAGACAGAGATCAATTTTCTGCTGAAGCAGGCCCTGAC |
| | | CATCGTGGGCACACTGCCTTTCACCTATATGCTGGAGAAGTGGCGC |
| | | TGGATGGTGTTTAAGGGCGAGATCCCAAAGGACCAGTGGATGAAGA |
| | | AGTGGTGGGAGATGAAGAGGGAGATCGTGGGAGTGGTGGAGCCAGT |
| | | GCCACACGACGAGACATACTGTGATCCAGCCTCCCTGTTCCATGTG |
| | | AGCAACGACTATTCTTTTATCAGGTACTATACCCGGACACTGTACC |
| | | AGTTCCAGTTTCAGGAGGCCCTGTGCCAGGCTGCTAAGCACGAGGG |
| | | ACCACTGCATAAGTGTGATATCTCTAATTCCACAGAGGCTGGCCAG |
| | | AAGCTGTTCAACATGCTGCGGCTGGGCAAGTCCGAGCCTTGGACCC |
| | | TGGCCCTGGAGAATGTGGTGGGCGCTAAGAATATGAACGTGAGGCC |
| | | CCTGCTGAACTATTTCGAGCCCCTGTTTACATGGCTGAAGGACCAG |
| | | AATAAGAACAGCTTTGTGGGCTGGTCCACCGATTGGAGCCCTTACG |
| | | CTAGCGGAGGAGGAGGAGAGCCAAAGAGCTCTGACAAGACCCACAC |
| | | ATGCCCCCCTTGTCCTGCTCCAGAGCTGCTGGGAGGACCATCCGTG |
| | | TTCCTGTTTCCACCCAAGCCTAAGGATACACTGATGATCTCCCGGA |
| | | CACCAGAGGTGACCTGCGTGGTGGTGGACGTGAGCCACGAGGGACC |
| | | CGAGGTGAAGTTCAATTGGTACGTGGATGGCGTGGAGGTGCATAAC |
| | | GCCAAGACCAAGCCCCGGGAGGAGCAGTATAATTCTACATACAGAG |
| | | TGGTGTCCGTGCTGACCGTGCTGCATCAGGACTGGCTGAATGGCAA |
| | | GGAGTACAAGTGCAAGGTGTCTAACAAGGCCCTGCCCGCTCCTATC |
| | | GAGAAGACAATCTCCAAGGCTAAGGGACAGCCTAGGGAGCCACAGG |
| | | TGTATACCCTGCCTCCAAGCAGGGACGAGCTGACAAAGAATCAGGT |
| | | GTCTCTGACCTGTCTGGTGAAGGGCTTCTACCCCTCTGATATCGCT |
| | | GTGGAGTGGGAGTCCAACGGCCAGCCTGAGAACAATTATAAGACCA |
| | | CACCCCCTGTGCTGGACAGCGATGGCTCTTTCTTTCTGTACTCCAA |
| | | GCTGACCGTGGATAAGAGCAGATGGCAGCAGGGCAACGTGTTTAGC |
| | | TGTTCTGTGATGCACGAAGCACTGCACAACCATTACACTCAGAAAT |
| | | CCCTGTCCCTGTCCCCTGGC |
| 31 | ACE2m4-hinge2CS- | ATGTCCTCCTCAAGCTGGCTGCTGCTGTCTCTGGTGGCCGTGACCG |
| | SG4-Fc | CCGCTCAGTCAACAATCGAAGAACAGGCAAAATACTTCCTGGACAA |
| | (exemplary DNA | GTTCAACCACGAGGCCGAGGATCTGTTTTATCAGTCCTCCCTGGCC |
| | sequence) | TCCTGGAACTACAATACCAACATCACAGAGGAGAATGTGCAGAACA |
| | | TGAACAATGCCGGCGACAAGTGGTCCGCCTTCCTGAAGGAGCAGTC |
| | | TACACTGGCCCAGATGTACCCCCTGCAGGAGATCCAGAATCTGATC |
| | | GTGAAGCTGCAGCTGCAGGCTCTGCAGCAGAACGGCTCTTCCGTGC |
| | | TGTCCGAGGATAAGAGCAAGCGCCTGAATACAATCCTGAACACCAT |
| | | GTCTACAATCTATTCCACCGGCAAGGTGTGCAATCCAGACAACCCC |
| | | CAGGAGTGTCTGCTGCTGGAGCCAGGCCTGAATGAGATCATGGCCA |
| | | ACTCTCTGGATTACAATGAGAGACTGTGGGCTTGGGAGTCCTGGCG |
| | | CAGCGAAGTGGGCAAGCAGCTGAGGCCCCTGTACGAGGAGTATGTG |
| | | GTGCTGAAGAACGAGATGGCCCGGGCTAATCACTACGAGGACTATG |
| | | GCGATTACTGGAGAGGCGACTACGAAGTGAATGGCGTGGACGGCTA |
| | | CGATTATTCTCGCGGCCAGCTGATCGAGGATGTGGAGCATACCTTT |
| | | GAGGAGATCAAGCCTCTGTATGAGCACCTGCATGCCTACGTGAGGG |
| | | CTAAGCTGATGAACGCCTATCCCTCTTACATCTCCCCTATCGGATG |
| | | CCTGCCAGCTCACCTGCTGGGCGACATGTGGGGCCGGTTCTGGACA |
| | | AATCTGTACTCCCTGACCGTGCCCTTTGGCCAGAAGCCTAACATCG |
| | | ACGTGACCGATGCCATGGTGGACCAGGCCTGGGATGCTCAGAGAAT |
| | | CTTCAAGGAGGCTGAGAAGTTCTTTGTGAGCGTGGGCCTGCCTAAC |
| | | ATGACACAGGGCTTTTGGGAGTACTCCATGCTGACCGACCCAGGCA |
| | | ACGTGCAGAAGGCCGTGTGCCACCCCACAGCTTGGGACCTGGGCAA |
| | | GGGCGATTTCCGCATCCTGATGTGCACCAAGGTGACAATGGACGAT |
| | | TTTCTGACCGCCCACCATGAGATGGGCCATATCCAGTATGATATGG |
| | | CTTACGCCGCTCAGCCATTCCTGCTGAGGAATGGCGCCAACGAGGG |
| | | CTTTCACGAGGCTGTGGGCGAGATCATGTCCCTGAGCGCCGCTACA |
| | | CCTAAGCATCTGAAGTCCATCGGCCTGCTGAGCCCAGACTTCCAGG |
| | | AGGATAACGAGACAGAGATCAATTTTCTGCTGAAGCAGGCCCTGAC |
| | | CATCGTGGGCACACTGCCTTTCACCTATATGCTGGAGAAGTGGCGC |
| | | TGGATGGTGTTTAAGGGCGAGATCCCAAAGGACCAGTGGATGAAGA |
| | | AGTGGTGGGAGATGAAGAGGGAGATCGTGGGAGTGGTGGAGCCAGT |
| | | GCCACACGACGAGACATACTGTGATCCAGCCTCCCTGTTCCATGTG |
| | | AGCAACGACTATTCTTTTATCAGGTACTATACCCGGACACTGTACC |
| | | AGTTCCAGTTTCAGGAGGCCCTGTGCCAGGCTGCTAAGCACGAGGG |
| | | ACCACTGCATAAGTGTGATATCTCTAATTCCACAGAGGCTGGCCAG |
| | | AAGCTGTTCAACATGCTGCGGCTGGGCAAGTCCGAGCCTTGGACCC |
| | | TGGCCCTGGAGAATGTGGTGGGCGCTAAGAATATGAACGTGAGGCC |
| | | CCTGCTGAACTATTTCGAGCCCCTGTTTACATGGCTGAAGGACCAG |
| | | AATAAGAACAGCTTTGTGGGCTGGTCCACCGATTGGAGCCCTTACG |
| | | CTAGCGGAGGAGGAGGAGAGCCAAAGAGCTCTGACAAGACCCACAC |
| | | AAGCCCCCCTAGCCCTGCTCCAGAGCTGCTGGGAGGACCATCCGTG |
| | | TTCCTGTTTCCACCCAAGCCTAAGGATACACTGATGATCTCCCGGA |
| | | CACCAGAGGTGACCTGCGTGGTGGTGGACGTGAGCCACGAGGGACC |
| | | CGAGGTGAAGTTCAATTGGTACGTGGATGGCGTGGAGGTGCATAAC |
| | | GCCAAGACCAAGCCCCGGGAGGAGCAGTATAATTCTACATACAGAG |
| | | TGGTGTCCGTGCTGACCGTGCTGCATCAGGACTGGCTGAATGGCAA |
| | | GGAGTACAAGTGCAAGGTGTCTAACAAGGCCCTGCCCGCTCCTATC |
| | | GAGAAGACAATCTCCAAGGCTAAGGGACAGCCTAGGGAGCCACAGG |
| | | TGTATACCCTGCCTCCAAGCAGGGACGAGCTGACAAAGAATCAGGT |
| | | GTCTCTGACCTGTCTGGTGAAGGGCTTCTACCCCTCTGATATCGCT |
| | | GTGGAGTGGGAGTCCAACGGCCAGCCTGAGAACAATTATAAGACCA |
| | | CACCCCCTGTGCTGGACAGCGATGGCTCTTTCTTTCTGTACTCCAA |
| | | GCTGACCGTGGATAAGAGCAGATGGCAGCAGGGCAACGTGTTTAGC |
| | | TGTTCTGTGATGCACGAAGCACTGCACAACCATTACACTCAGAAAT |
| | | CCCTGTCCCTGTCCCCTGGC |

Signal peptides are italics-underlined, if shown, and are not required in the final protein product.

### REFERENCES

The content of each of the following references is hereby incorporated by reference in its entirety.

### References

[1] Hrenak J, Simko F. Renin-Angiotensin System: An Important Player in the Pathogenesis of Acute Respiratory Distress Syndrome. Int J Mol Sci. 2020; 21.
[2] Gao Y L, Du Y, Zhang C, Cheng C, Yang H Y, Jin Y F, et al. Role of Renin-Angiotensin System in Acute Lung Injury Caused by Viral Infection. Infect Drug Resist. 2020; 13:3715-25.
[3] Choudhary S, Sharma K, Silakari O. The interplay between inflammatory pathways and COVID-19: A critical review on pathogenesis and therapeutic options. Microb Pathog. 2020; 150:104673.
[4] Yan R, Zhang Y, Li Y, Xia L, Guo Y, Zhou Q. Structural basis for the recognition of SARS-CoV-2 by full-length human ACE2. Science. 2020; 367:1444-8.
[5] Wrapp D, Wang N, Corbett K S, Goldsmith J A, Hsieh C L, Abiona O, et al. Cryo-EM structure of the 2019-nCoV spike in the prefusion conformation. Science. 2020.
[6] Hoffmann M, Kleine-Weber H, Schroeder S, Kruger N, Herrler T, Erichsen S, et al. SARS-CoV-2 Cell Entry Depends on ACE2 and TMPRSS2 and Is Blocked by a Clinically Proven Protease Inhibitor. Cell. 2020; 181:271-80 e8.
[7] Tegally H, Wilkinson E, Giovanetti M, Iranzadeh A, Fonseca V, Giandhari J, et al. Emergence and rapid spread of a new severe acute respiratory syndrome-related coronavirus 2 (SARS-CoV-2) lineage with multiple spike mutations in South Africa. medRxiv. 2020:2020.12.21.20248640.
[8] Zhou F, Yu T, Du R, Fan G, Liu Y, Liu Z, et al. Clinical course and risk factors for mortality of adult inpatients with COVID-19 in Wuhan, China: a retrospective cohort study. Lancet. 2020; 395:1054-62.
[9] Yin S, Huang M, Li D, Tang N. Difference of coagulation features between severe pneumonia induced by SARS-CoV2 and non-SARS-CoV2. J Thromb Thrombolysis. 2020.
[10] Zou Z, Yan Y, Shu Y, Gao R, Sun Y, Li X, et al. Angiotensin-converting enzyme 2 protects from lethal avian influenza A H5N1 infections. Nat Commun. 2014; 5:3594.
[11] Huang F, Guo J, Zou Z, Liu J, Cao B, Zhang S, et al. Angiotensin II plasma levels are linked to disease severity and predict fatal outcomes in H7N9-infected patients. Nat Commun. 2014; 5:3595.
[12] Monteil V, Kwon H, Prado P, Hagelkruys A, Wimmer R A, Stahl M, et al. Inhibition of SARS-CoV-2 Infections in Engineered Human Tissues Using Clinical-Grade Soluble Human ACE2. Cell. 2020; 181:905-13 e7.
[13] Lei C, Qian K, Li T, Zhang S, Fu W, Ding M, et al. Neutralization of SARS-CoV-2 spike pseudotyped virus by recombinant ACE2-Ig. Nat Commun. 2020; 11:2070.
[14] Chan K K, Dorosky D, Sharma P, Abbasi S A, Dye J M, Kranz D M, et al. Engineering human ACE2 to optimize binding to the spike protein of SARS coronavirus 2. Science. 2020; 369:1261-5.
[15] Glasgow A, Glasgow J, Limonta D, Solomon P, Lui I, Zhang Y, et al. Engineered ACE2 receptor traps potently neutralize SARS-CoV-2. Proc Natl Acad Sci USA. 2020; 117:28046-55.
[16] Altschul S F, Gish W, Miller W, Myers E W, Lipman D J. Basic local alignment search tool. J Mol Biol. 1990; 215:403-10.
[17] Altschul S F, Madden T L, Schaffer A A, Zhang J, Zhang Z, Miller W, et al. Gapped BLAST and PSI-BLAST: a new generation of protein database search programs. Nucl Acids Res. 1997; 25:3389-402.
[18] Myers E W, Miller W. Optimal alignments in linear space. Comput Appl Biosci. 1988; 4:11-7.
[19] Eisenberg D, McLachlan A D. Solvation energy in protein folding and binding. Nature. 1986; 319:199-203.
[20] Tanokura M. 1H-NMR study on the tautomerism of the imidazole ring of histidine residues. I. Microscopic pK values and molar ratios of tautomers in histidine-containing peptides. Biochim Biophys Acta. 1983; 742:576-85.
[21] Giudicelli V, Brochet X, Lefranc M P. IMGT/V-QUEST: IMGT standardized analysis of the immunoglobulin (IG) and T cell receptor (TR) nucleotide sequences. Cold Spring Harb Protoc. 2011; 2011:695-715.
[22] Giudicelli V, Duroux P, Ginestoux C, Folch G, Jabado-Michaloud J, Chaume D, et al. IMGT/LIGM-DB, the IMGT comprehensive database of immunoglobulin and T cell receptor nucleotide sequences. Nucleic Acids Res. 2006; 34:D781-4.
[23] Lefranc M P, Giudicelli V, Duroux P, Jabado-Michaloud J, Folch G, Aouinti S, et al. IMGT(R), the international ImMunoGeneTics information system(R) 25 years on. Nucleic Acids Res. 2015; 43:D413-22.
[24] Lippi G, Lavie C J, Henry B M, Sanchis-Gomar F. Do genetic polymorphisms in angiotensin converting enzyme 2 (ACE2) gene play a role in coronavirus disease 2019 (COVID-19)? Clin Chem Lab Med. 2020; 58:1415-22.
[25] Sorokina M, J MCT, Barrera-Vilarmau S, Paschke R, Papasotiriou I, Rodrigues J, et al. Structural models of human ACE2 variants with SARS-CoV-2 Spike protein for structure-based drug design. Sci Data. 2020; 7:309.
[26] Stawiski E W, Diwanji D, Suryamohan K, Gupta R, Fellouse F A, Sathirapongsasuti J F, et al. Human ACE2 receptor polymorphisms predict SARS-CoV-2 susceptibility. bioRxiv. 2020:2020.04.07.024752.
[27] Vaniotis G, Moffett S, Sulea T, Wang N, Elahi S M, Lessard E, et al. Enhanced anti-metastatic bioactivity of an IGF-TRAP re-engineered to improve physicochemical properties. Sci Rep. 2018; 8:17361.
[28] Strand J, Huang C T, Xu J. Characterization of Fc-fusion protein aggregates derived from extracellular domain disulfide bond rearrangements. J Pharm Sci. 2013; 102:441-53.
[29] Bournazos S, Gupta A, Ravetch J V. The role of IgG Fc receptors in antibody-dependent enhancement. Nat Rev Immunol. 2020; 20:633-43.
[30] Fan E, Brodie D, Slutsky A S. Acute Respiratory Distress Syndrome: Advances in Diagnosis and Treatment. JAMA. 2018; 319:698-710.
[31] Ragab D, Salah Eldin H, Taeimah M, Khattab R, Salem R. The COVID-19 Cytokine Storm; What We Know So Far. Front Immunol. 2020; 11:1446.
[32] Lee C, Choi W J. Overview of COVID-19 inflammatory pathogenesis from the therapeutic perspective. Arch Pharm Res. 2021.
[33] Cornell W D, Cieplak P, Bayly C I, Gould I R, Merz K M, Ferguson D M, et al. A Second Generation Force Field for the Simulation of Proteins, Nucleic Acids, and Organic Molecules. J Am Chem Soc. 1995; 117:5179-97.
[34] Hornak V, Abel R, Okur A, Strockbine B, Roitberg A, Simmerling C. Comparison of multiple Amber force fields and development of improved protein backbone parameters. Proteins. 2006; 65:712-25.
[35] Vivcharuk V, Baardsnes J, Deprez C, Sulea T, Jaramillo M, Corbeil C R, et al. Assisted Design of Antibody and Protein Therapeutics (ADAPT). PLoS One. 2017; 12:e0181490.
[36] Sulea T, Hussack G, Ryan S, Tanha J, Purisima E O. Application of Assisted Design of Antibody and Protein Therapeutics (ADAPT) improves efficacy of a Clostridium difficile toxin A single-domain antibody. Sci Rep. 2018; 8:2260.
[37] Sulea T, Vivcharuk V, Corbeil C R, Deprez C, Purisima E O. Assessment of Solvated Interaction Energy Function for Ranking Antibody-Antigen Binding Affinities. J Chem Inf Model. 2016; 56:1292-303.
[38] Durocher Y, Perret S, Kamen A. High-level and high-throughput recombinant protein production by transient transfection of suspension-growing human 293-EBNA1 cells. Nucl Acids Res. 2002; 30:e9.
[39] Voronov S, Zueva N, Orlov V, Arutyunyan A, Kost O. Temperature-induced selective death of the C-domain within angiotensin-converting enzyme molecule. FEBS Lett. 2002; 522:77-82.
[40] Xiao F, Burns K D. Measurement of Angiotensin Converting Enzyme 2 Activity in Biological Fluid (ACE2). Methods Mol Biol. 2017; 1527:101-15.
[41] Xiao F, Zimpelmann J, Agaybi S, Gurley S B, Puente L, Burns K D. Characterization of angiotensin-converting enzyme 2 ectodomain shedding from mouse proximal tubular cells. PLoS One. 2014; 9:e85958.
[42] Colwill K, Galipeau Y, Stuible M, Gervais C, Arnold C, Rathod B, et al. A “Made-in-Canada” serology solution for profiling humoral immune responses to SARS-CoV-2 infection and vaccination. medRxiv. 2021:2021.10.25.21265476.
[43] Abe K T, Li Z, Samson R, Samavarchi-Tehrani P, Valcourt E J, Wood H, et al. A simple protein-based surrogate neutralization assay for SARS-CoV-2. JCI Insight. 2020; 5.
[44] Starr T N, Greaney A J, Hilton S K, Ellis D, Crawford K H D, Dingens A S, et al. Deep Mutational Scanning of SARS-CoV-2 Receptor Binding Domain Reveals Constraints on Folding and ACE2 Binding. Cell. 2020; 182:1295-310 e20.
[45] Crawford K H D, Eguia R, Dingens A S, Loes A N, Malone K D, Wolf C R, et al. Protocol and Reagents for Pseudotyping Lentiviral Particles with SARS-CoV-2 Spike Protein for Neutralization Assays. Viruses. 2020; 12.
[46] Cote J, Garnier A, Massie B, Kamen A. Serum-free production of recombinant proteins and adenoviral vectors by 293SF-3F6 cells. Biotechnol Bioeng. 1998; 59:567-75.
[47] Baum A, Ajithdoss D, Copin R, Zhou A, Lanza K, Negron N, et al. REGN-COV2 antibodies prevent and treat SARS-CoV-2 infection in rhesus macaques and hamsters. Science. 2020; 370:1110-5.
[48] Callera G E, Antunes T T, Correa J W, Moorman D, Gutsol A, He Y, et al. Differential renal effects of candesartan at high and ultra-high doses in diabetic mice-potential role of the ACE2/AT2R/Mas axis. Biosci Rep. 2016; 36.
[49] Imai M, Iwatsuki-Horimoto K, Hatta M, Loeber S, Halfmann P J, Nakajima N, et al. Syrian hamsters as a small animal model for SARS-CoV-2 infection and countermeasure development. Proc Natl Acad Sci USA. 2020; 117:16587-95.
[50] Akache B, Renner T M, Tran A, Deschatelets L, Dudani R, Harrison B A, et al. Immunogenic and efficacious SARS-CoV-2 vaccine based on resistin-trimerized spike antigen SmT1 and SLA archaeosome adjuvant. Scientific reports. 2021; 11:21849.

1. A polypeptide construct capable of neutralizing SARS-CoV-2 and converting Ang-I I to Ang-(1-7), the polypeptide construct comprising four regions and having the general formula:
R1[hACE2I⁹²(18-614),X²⁷,X²⁶¹,X³³⁰]-R2-R3[HingeS²²⁰,X²²⁶,X²²⁹]-R4[C_{H}2G²⁷⁰-C_{H}3]
and where:
R1, denoted hACE2I⁹²(18-614),X²⁷,X²⁶¹X3³⁰, is the N-terminal first region comprising the naturally occurring variant Ile92 (I92) of the human angiotensin converting enzyme 2 (hACE2) receptor catalytic domain residues 18 to 614 comprising residues X²⁷, X²⁶¹ and X³³⁰; wherein X²⁷ is the amino-acid residue at position 27 that is either Thr or Tyr, X²⁶¹ is the amino-acid residue at position 261 that is either Cys or Ser, and X³³⁰ is the amino-acid residue at position 330 that is either Asn or Tyr;
R2 is a second region comprising a flexible peptide spacer;
R3 is a third region comprising the hinge region of a human IgG1 heavy chain antibody, wherein said hinge region comprises residues S²²⁰, X²²⁶ and X²²⁹; wherein the amino acid residue at human IgG1 hinge position 220 is Ser, and wherein the amino acid residues at human IgG1 hinge positions 226 and 229 are either Cys or Ser; and
R4 is a fourth region at the C-terminus of the polypeptide wherein R4 is denoted as and comprises C_{H}2G²⁷⁰-C_{H}3, and comprises the second constant domain (C_{H}2) and the third constant domain (C_{H}3) of human IgG1 antibody heavy chain, where the amino acid residue at position 270 in the human IgG1 C_{H}2 domain is Glycine.

2. The polypeptide construct according to claim 1, wherein said flexible peptide spacer comprises Gly and Ser residues.

3. The polypeptide construct according to claim 1, wherein said polypeptide construct neutralizes a cellular infection mediated by a SARS-CoV-2 spike protein variant with an IC50 below the concentration of 2500 ng/mL (12.5 nM).

4. The polypeptide construct according to claim 1, wherein said polypeptide construct neutralizes the SARS-CoV-2 with an IC50 of at least 500 ng/mL (2.5 nM).

5. The polypeptide construct according to claim 1, wherein said polypeptide construct retains at least 30% of the catalytic efficiency (k_{cat}/K_{M}) and at least 60% of the enzymatic activity of recombinant human ACE2.

6. The polypeptide construct according to claim 1, wherein R1 comprises a sequence selected from the group consisting of SEQ ID NO:2, SEQ ID NO:4, SEQ ID NO:5, SEQ ID NO:6, SEQ ID NO:7 and a sequence at least 90% identical thereto.

7. The polypeptide construct according to claim 1, wherein R2 comprises a sequence selected from the group consisting of SEQ ID NO:8, SEQ ID NO:9, and a sequence at least 90% identical thereto.

8. The polypeptide construct according to claim 1, wherein R3 comprises a sequence selected from the group consisting of SEQ ID NO:11, SEQ ID NO:12, and a sequence at least 90% identical thereto.

9. The polypeptide construct according to claim 1, wherein R4 comprises a sequence having SEQ ID NO:14, or a sequence at least 90% identical thereto.

10. The polypeptide construct according to claim 1, comprising a sequence selected from the group consisting of SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:28, and a sequence at least 90% identical thereto.

11. The polypeptide construct according to claim 1, wherein the construct is a dimeric polypeptide.

12. The polypeptide construct according to claim 11, wherein the dimeric polypeptide may be linked or may dimerize via the respective R3 hinge regions by disulfide bridges.

13. A nucleic acid molecule encoding the polypeptide construct of claim 1.

14. A vector comprising the nucleic acid molecule of claim 12.

15. A nucleic acid molecule that encodes the polypeptide of claim 1 in a form that is secretable by a selected expression host.

16. A composition comprising the polypeptide construct of claim 1 and a pharmaceutically-acceptable carrier, diluent, or excipient.

17. A transgenic cellular host comprising the nucleic acid molecule of claim 13.

18. The transgenic cellular host of claim 17, further comprising a second nucleic acid molecule encoding a second polypeptide construct the same as the first polypeptide construct.

19. A method for producing a dimeric polypeptide comprising culturing the host of claim 17 and recovering from a medium conditioned by the growth of that host the dimeric polypeptide construct comprising a sequence selected from the group consisting of SEQ ID NO:16, SEQ ID NO:19, SEQ ID NO:20, SEQ ID NO:21, SEQ ID NO:22, SEQ ID NO:23, SEQ ID NO:24, SEQ ID NO:25, SEQ ID NO:28, and a sequence at least 90% identical thereto.

20. (canceled)

21. A method of treating a coronaviral infection, acute respiratory distress syndrome (ARDS), or major organ failure associated with COVID-19, the method comprising administering to a subject in need thereof the polypeptide construct according to claim 1.


SEQUENCE LISTING (31 sequences)

SEQ ID NO: 1 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 2 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 3 - protein, 597 aa, Artificial sequence
  Mutant hACE2 (T27Y, N330Y)
QSTIEEQAKYFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 4 - protein, 597 aa, Artificial sequence
  Mutant hACE2I92 (T27Y)
QSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 5 - protein, 597 aa, Artificial sequence
  Mutant hACE2I92 (N330Y)
QSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 6 - protein, 597 aa, Artificial sequence
  Mutant hACE2I92 (T27Y, N330Y)
QSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 7 - protein, 597 aa, Artificial sequence
  Mutant hACE2I92 (T27Y, C261S, N330Y)
QSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGSLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 8 - protein, 5 aa, Artificial sequence
  Peptide spacer SG4
SGGGG

SEQ ID NO: 9 - protein, 15 aa, Artificial sequence
  Peptide spacer SG4x3
SGGGGSGGGGSGGGG

SEQ ID NO: 10 - protein, 15 aa, Homo sapiens
EPKSCDKTHTCPPCP

SEQ ID NO: 11 - protein, 15 aa, Artificial sequence
  Mutant human IgG1 hinge sequence
EPKSSDKTHTCPPCP

SEQ ID NO: 12 - protein, 15 aa, Artificial sequence
  Mutant human IgG1 hinge sequence
EPKSSDKTHTSPPSP

SEQ ID NO: 13 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 216 aa, Artificial sequence
  Mutant IgG1 CH2-CH3 sequence (G270)
APELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 850 aa, Artificial sequence
  hACE2-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 850 aa, Artificial sequence
  hACE2I92-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 860 aa, Artificial sequence
  hACE2-SG4x3-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGSGGGGSGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 850 aa, Artificial sequence
  hACE2m1-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 850 aa, Artificial sequence
  hACE2m2-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 850 aa, Artificial sequence
  hACE2m3-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 850 aa, Artificial sequence
  hACE2m4-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 850 aa, Artificial sequence
  hACE2m4-C261S-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGSLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTCPPCPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 850 aa, Artificial sequence
  hACE2m4-hinge2CS-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 850 aa, Artificial sequence
  hACE2m3-hinge2CS-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 850 aa, Artificial sequence
  hACE2m2-hinge2CS-SG4-Fc fusion
MSSSSWLLLSLVAVTAAQSTIEEQAKYFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 850 aa, Artificial sequence
  hACE2m1-hinge2CS-SG4-Fc
MSSSSWLLLSLVAVTAAQSTIEEQAKYFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 850 aa, Artificial sequence
  hACE2-hinge2CS-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 850 aa, Artificial sequence
  hACE2I92-hinge2CS-SG4-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLIVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 860 aa, Artificial sequence
  hACE2-hinge2CS-SG4x3-Fc fusion
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYASGGGGSGGGGSGGGGEPKSSDKTHTSPPSPAPELLGGPSVFLFPPKPKDTLMISRTPEVTCVVVDVSHEGPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 2550 bp, Artificial sequence
  hACE2m4-SG4-Fc fusion (exemplary DNA sequence)
atgtcctcctcaagctggctgctgctgtctctggtggccgtgaccgccgctcagtcaacaatcgaagaacaggcaaaatacttcctggacaagttcaaccacgaggccgaggatctgttttatcagtcctccctggcctcctggaactacaataccaacatcacagaggagaatgtgcagaacatgaacaatgccggcgacaagtggtccgccttcctgaaggagcagtctacactggcccagatgtaccccctgcaggagatccagaatctgatcgtgaagctgcagctgcaggctctgcagcagaacggctcttccgtgctgtccgaggataagagcaagcgcctgaatacaatcctgaacaccatgtctacaatctattccaccggcaaggtgtgcaatccagacaacccccaggagtgtctgctgctggagccaggcctgaatgagatcatggccaactctctggattacaatgagagactgtgggcttgggagtcctggcgcagcgaagtgggcaagcagctgaggcccctgtacgaggagtatgtggtgctgaagaacgagatggcccgggctaatcactacgaggactatggcgattactggagaggcgactacgaagtgaatggcgtggacggctacgattattctcgcggccagctgatcgaggatgtggagcatacctttgaggagatcaagcctctgtatgagcacctgcatgcctacgtgagggctaagctgatgaacgcctatccctcttacatctcccctatcggatgcctgccagctcacctgctgggcgacatgtggggccggttctggacaaatctgtactccctgaccgtgccctttggccagaagcctaacatcgacgtgaccgatgccatggtggaccaggcctgggatgctcagagaatcttcaaggaggctgagaagttctttgtgagcgtgggcctgcctaacatgacacagggcttttgggagtactccatgctgaccgacccaggcaacgtgcagaaggccgtgtgccaccccacagcttgggacctgggcaagggcgatttccgcatcctgatgtgcaccaaggtgacaatggacgattttctgaccgcccaccatgagatgggccatatccagtatgatatggcttacgccgctcagccattcctgctgaggaatggcgccaacgagggctttcacgaggctgtgggcgagatcatgtccctgagcgccgctacacctaagcatctgaagtccatcggcctgctgagcccagacttccaggaggataacgagacagagatcaattttctgctgaagcaggccctgaccatcgtgggcacactgcctttcacctatatgctggagaagtggcgctggatggtgtttaagggcgagatcccaaaggaccagtggatgaagaagtggtgggagatgaagagggagatcgtgggagtggtggagccagtgccacacgacgagacatactgtgatccagcctccctgttccatgtgagcaacgactattcttttatcaggtactatacccggacactgtaccagttccagtttcaggaggccctgtgccaggctgctaagcacgagggaccactgcataagtgtgatatctctaattccacagaggctggccagaagctgttcaacatgctgcggctgggcaagtccgagccttggaccctggccctggagaatgtggtgggcgctaagaatatgaacgtgaggcccctgctgaactatttcgagcccctgtttacatggctgaaggaccagaataagaacagctttgtgggctggtccaccgattggagcccttacgctagcggaggaggaggagagccaaagagctctgacaagacccacacatgccccccttgtcctgctccagagctgctgggaggaccatccgtgttcctgtttccacccaagcctaaggatacactgatgatctcccggacaccagaggtgacctgcgtggtggtggacgtgagccacgagggacccgaggtgaagttcaattggtacgtggatggcgtggaggtgcataacgccaagaccaagccccgggaggagcagtataattctacatacagagtggtgtccgtgctgaccgtgctgcatcaggactggctgaatggcaaggagtacaagtgcaaggtgtctaacaaggccctgcccgctcctatcgagaagacaatctccaaggctaagggacagcctagggagccacaggtgtataccctgcctccaagcagggacgagctgacaaagaatcaggtgtctctgacctgtctggtgaagggcttctacccctctgatatcgctgtggagtgggagtccaacggccagcctgagaacaattataagaccacaccccctgtgctggacagcgatggctctttctttctgtactccaagctgaccgtggataagagcagatggcagcagggcaacgtgtttagctgttctgtgatgcacgaagcactgcacaaccattacactcagaaatccctgtccctgtcccctggc

SEQ ID NO: 31 - DNA, 2550 bp, Artificial sequence
  hACE2m4-hinge2CS-SG4-Fc fusion (exemplary DNA sequence)
atgtcctcctcaagctggctgctgctgtctctggtggccgtgaccgccgctcagtcaacaatcgaagaacaggcaaaatacttcctggacaagttcaaccacgaggccgaggatctgttttatcagtcctccctggcctcctggaactacaataccaacatcacagaggagaatgtgcagaacatgaacaatgccggcgacaagtggtccgccttcctgaaggagcagtctacactggcccagatgtaccccctgcaggagatccagaatctgatcgtgaagctgcagctgcaggctctgcagcagaacggctcttccgtgctgtccgaggataagagcaagcgcctgaatacaatcctgaacaccatgtctacaatctattccaccggcaaggtgtgcaatccagacaacccccaggagtgtctgctgctggagccaggcctgaatgagatcatggccaactctctggattacaatgagagactgtgggcttgggagtcctggcgcagcgaagtgggcaagcagctgaggcccctgtacgaggagtatgtggtgctgaagaacgagatggcccgggctaatcactacgaggactatggcgattactggagaggcgactacgaagtgaatggcgtggacggctacgattattctcgcggccagctgatcgaggatgtggagcatacctttgaggagatcaagcctctgtatgagcacctgcatgcctacgtgagggctaagctgatgaacgcctatccctcttacatctcccctatcggatgcctgccagctcacctgctgggcgacatgtggggccggttctggacaaatctgtactccctgaccgtgccctttggccagaagcctaacatcgacgtgaccgatgccatggtggaccaggcctgggatgctcagagaatcttcaaggaggctgagaagttctttgtgagcgtgggcctgcctaacatgacacagggcttttgggagtactccatgctgaccgacccaggcaacgtgcagaaggccgtgtgccaccccacagcttgggacctgggcaagggcgatttccgcatcctgatgtgcaccaaggtgacaatggacgattttctgaccgcccaccatgagatgggccatatccagtatgatatggcttacgccgctcagccattcctgctgaggaatggcgccaacgagggctttcacgaggctgtgggcgagatcatgtccctgagcgccgctacacctaagcatctgaagtccatcggcctgctgagcccagacttccaggaggataacgagacagagatcaattttctgctgaagcaggccctgaccatcgtgggcacactgcctttcacctatatgctggagaagtggcgctggatggtgtttaagggcgagatcccaaaggaccagtggatgaagaagtggtgggagatgaagagggagatcgtgggagtggtggagccagtgccacacgacgagacatactgtgatccagcctccctgttccatgtgagcaacgactattcttttatcaggtactatacccggacactgtaccagttccagtttcaggaggccctgtgccaggctgctaagcacgagggaccactgcataagtgtgatatctctaattccacagaggctggccagaagctgttcaacatgctgcggctgggcaagtccgagccttggaccctggccctggagaatgtggtgggcgctaagaatatgaacgtgaggcccctgctgaactatttcgagcccctgtttacatggctgaaggaccagaataagaacagctttgtgggctggtccaccgattggagcccttacgctagcggaggaggaggagagccaaagagctctgacaagacccacacaagcccccctagccctgctccagagctgctgggaggaccatccgtgttcctgtttccacccaagcctaaggatacactgatgatctcccggacaccagaggtgacctgcgtggtggtggacgtgagccacgagggacccgaggtgaagttcaattggtacgtggatggcgtggaggtgcataacgccaagaccaagccccgggaggagcagtataattctacatacagagtggtgtccgtgctgaccgtgctgcatcaggactggctgaatggcaaggagtacaagtgcaaggtgtctaacaaggccctgcccgctcctatcgagaagacaatctccaaggctaagggacagcctagggagccacaggtgtataccctgcctccaagcagggacgagctgacaaagaatcaggtgtctctgacctgtctggtgaagggcttctacccctctgatatcgctgtggagtgggagtccaacggccagcctgagaacaattataagaccacaccccctgtgctggacagcgatggctctttctttctgtactccaagctgaccgtggataagagcagatggcagcagggcaacgtgtttagctgttctgtgatgcacgaagcactgcacaaccattacactcagaaatccctgtccctgtcccctggc